(19) United States
(12) Patent Application Publication
(10) Pub. No.: US 2025/0302974 A1
(43) Pub. Date: Oct. 2, 2025

(54) LECTIN-DRUG CONJUGATES

(71) Applicant: UNICHEM LABORATORIES LIMITED, Mumbai, Maharashtra (IN)

(72) Inventors: Dhananjay Sathe, Thane (IN); Saravanakumar Iyappan, Hyderabad (IN); Gautam Bakshi, Ambala City (IN); Ganesh Patil, Navi Mumbai (IN)

(21) Appl. No.: 18/692,059

(22) PCT Filed: Sep. 30, 2022
(86) PCT No.: PCT/IB2022/059340

§ 371 Date: Mar. 14, 2024

(30) Foreign Application Priority Data
Oct. 1, 2021 (IN) 202121044592

(51) Int. Cl. A61K 47/64; A61P 35/00; C07K 14/37

(52) U.S. Cl. CPC A61K 47/6415; A61P 35/00; C07K 14/37

(57) ABSTRACT

The present disclosure relates to protein-drug conjugates. The present disclosure specifically relates to the protein-drug conjugates, wherein the protein is a lectin protein having amino acid sequence of SEQ ID NO: 1; or lectin protein having at least 70% sequence identity to SEQ ID NO: 1. The disclosure further relates to process and composition of protein-drug conjugates and method of use thereof.

### REFERENCE TO AN ELECTRONIC SEQUENCE LISTING

The contents of the electronic “Sequence Listing” (Replacement Sequence Listing.xml, 10,882 bytes; Date of Creation: Oct. 8 2024) is herein incorporated by reference in its entirety.

### FIELD OF INVENTION

The present disclosure relates to protein-drug conjugates, more specifically the disclosure relates to the conjugates of lectin proteins and cytotoxic agents or therapeutic agent, their process of preparation and use in the method of treatment of cancer.

### BACKGROUND

Advances in the systemic therapy of cancer, including chemotherapy, hormonal therapy, targeted therapy, and immunotherapy have been responsible for improvements in cancer related mortality. Although such advancements have yet to benefit all cancer types, systemic therapies have led to an improvement in overall survival in both the adjuvant and metastatic setting for many cancers. With the pressure to make therapies available as soon as possible, the side-effects of systemic therapies, in particular long-term side-effects are not very well characterized and understood. Increasingly, a number of cancer types are requiring long-term and even lifelong systemic therapy. Because the incidence of cancer in the world will increase over the next several decades and there will be more people living with cancer, it is important to have an understanding of the potential side-effects of new systemic therapies.

Conventional chemotherapy agents were the first agents in the armamentarium for the war on cancer. Categories today include, but are not limited to, alkylating agents (e.g., cyclophosphamide, temozolomide, cisplatin, oxaliplatin), anti-metabolites (e.g., methotrexate, cytarabine, fluorouracil, capecitabine), anti-tumor antibiotics (e.g., doxorubicin, epirubicin, bleomycin), topoisomerase inhibitors (e.g., etoposide, irinotecan) and microtubule stabilizers (e.g., paclitaxel, docetaxel). Typically, these agents are not tumor specific and frequently their administration results in significant toxic effects to non-cancerous tissues. The success of these agents relies largely upon their differential toxicity for tumor cells which typically have a high mitotic rate and increased dependence on continuous supply of biomolecules for growth, compared to normal non-cancerous tissues. Present and future challenges include identifying the appropriate dosing of cytotoxic agents (and other components of multimodal anti-cancer treatment regimens) to maximize therapeutic efficacy while limiting both acute and long-term side-effects.

Targeted therapy or immunotherapy offers considerable advantages in delivering growth inhibitory or cytotoxic effects in a much more cell-specific manner. Furthermore, these drugs do not induce the same profile of acute toxicities such as myelosuppression and nausea and vomiting which accompany many of the conventional non-targeted cytotoxic drugs. As described below some of the newer targeted agents have proven highly effective anti-cancer treatments, and have already delivered startling improvements in survival for certain cancer. Examples of targeted therapies include Imatinib Tyrosine Kinase Inhibitor (TKI), and monoclonal antibodies such as trastuzumab, bevacizumab, panitumumab, and cetuximab. Targeted therapy also includes antibody drug conjugates which is a new class of biological drugs wherein the anticancer agent or other therapeutic agent are attached to the antibody with aid of linker. Some of the FDA approved antibody drug conjugates include Gemtuzumab ozogamicin, Brentuximab vedotin, Trastuzumab emtansine, inotizimab ozogamicin, Moxetumomab pasudotox, Polatuzumab vedotin-piiq, Enfotumab vedotin, Trastuzumab deruxtecan, Sacituzumab govitecan and Belantamab mafodotin.

To date, antibodies have proved to be effective therapeutics. These antibody therapeutics are used in much the same way as injected small-molecule chemotherapeutics.

Radioimmunotherapy provides further examples of the successful use of antibodies in cancer therapeutics. Unfortunately, despite the successful use of radiation-delivery antibody vehicles such as Zevalin and Bexxar, these antibody based drugs have serious shortcomings. For example, both are whole IgG molecules that remain in the circulation for days; they pass through the highly radiation-sensitive bone marrow throughout this period, and bone-marrow toxicity limits the dose of radiation that can be tolerated by patients.

Lectins are proteins or glycoproteins that are an important group of bioactive proteins found in nature. Lectins are used as tools for diagnostic and therapeutic purpose in health care areas. Lectins are also used in the purification of glycoproteins, oligosaccharide analysis and in cell selection processes. Lectins can bind with mono, di or oligosaccharides of the sugar moiety found in polysaccharides, glycoproteins or glycolipids. Lectins are specific to various monosaccharide or glycans and thus have enormous potential in the area of medical assays. Cancer cells are shown to express and/or secrete a variety of glycoconjugates with an aberrant glycan structure. Owing to their sugar specificity towards a particular linkage, lectins have an ability to differentiate changes in glycan structures in cancerous cell from their noncancerous counterparts, leading to their application in cancer diagnosis and treatment.

In the field of protein drug conjugates there are minimal studies which focus on lectin drug conjugates for the treatment of cancer. U.S. Pat. Nos. 9,504,756, and 6,214,345, describe antibody drug conjugates, and lectin is described as an alternative ligand moiety for targeted delivery of cytotoxic drugs for the treatment of cancer.

Indian application 1265/MUM/2004 explicitly discloses a drug or anticancer compound conjugate in which lectin obtained from a fungal culture of Sclerotium rolfsii (SR, S. rolfsii) and Rhizoctonia bataticola (RB) is used as carrier or conjugating protein for the transport of anticancer drug to the target cell.

Lectin conjugates, wherein lectin is a carrying agent or target seeking agent, for targeted delivery of cytotoxic drugs and methods of producing the same are disclosed in KR100638706B1, U.S. Pat. No. 7,015,313B2, U.S. Pat. No. 7,045,300B2 and US20060251580A1.

Plant based lectin has been explored for its targeting ability in the field of drug conjugates. Wheat germ agglutinin a plant lectin conjugated to Doxorubicin via acid liable cis-aconityl linker for delivery of prodrug Doxorubicin to the human colonocytes has been studied by Michel writh et al. (Michel writh et. al, Pharmaceutical research, Vol 15, NO. 7, 1998). Gunjan et al, studied the specific interaction of plant legume lectin such as Peanut agglutinin and concanavalin with Phycocyanin for the application of photodynamic therapy. Peanut agglutinin and concanavalin conjugated with Phycocyanin with the aid of chemical linker such as SPDP and studied for effective delivery of the drug. (Gunjan Pandey et al., Photochemistry and Photobiology, 85: 1126-1133, 2009,). Daichi Kitaguchi et al, studied the binding affinity of rBC2LCN, a lectin isolated from Burkholderia that binds cell surface glycans of colorectal cancer for effective delivery of Pseudomonas exotoxin (PE38) for the treatment of cancer. This study also reveals the therapeutic efficacy and toxicity of rBC2LCN-PE38 conjugate in Colo Rectal Cancer mouse xenograft models (Daichi Kitaguchi et al Cancer Science.; 111:4548-4557. 2020)

In the case of antibody drug conjugates, only a tiny amount of drug enters the cell, as the number of antigens present on cell surfaces is less and only one antibody interacts with antigen. Hence antibody drug conjugates need repeated dosage in order to achieve good therapeutic effect resulting in exorbitant treatment. Further even though the above references reveal conjugates of lectins, there are no such conjugates which are commercially available as of today. Lectins are highly specific towards glycans present on neoplastic tissues and are potentially better therapeutic ligands than monoclonal antibodies due to their cost-effectiveness, reduced toxicity and reduced side effects. There is a need for improved cancer treatments that are cost effective.

### SUMMARY

In one embodiment, the present disclosure provides a highly effective product for the treatment of cancer. In one embodiment is provided a recombinant lectin drug conjugate, wherein the recombinant lectin is derived from Sclerotium rolfsii lectin (SRL).

Also provided is a method of treatment or prevention of cancer in a subject in need thereof; wherein the method comprises use of therapeutically effective amount of recombinant lectin drug conjugate.

In one embodiment is provided the use of a recombinant lectin drug conjugate as a novel therapeutic agent for the treatment or prevention of cancer, wherein the recombinant lectin is derived from Sclerotium rolfsii lectin.

In one embodiment, is provided a composition comprising recombinant lectin drug conjugate for the treatment or prevention of cancer.

According to an aspect of the present disclosure, there is provided a compound of Formula I:

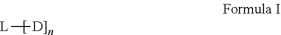

or a pharmaceutically acceptable salt thereof, wherein
n is 1 to 9;
L is a recombinant lectin derived from S. rolfsii;
D is selected from a cytotoxic agent, therapeutic agent or a compound comprising a cytotoxic agent or therapeutic agent;
wherein each D is conjugated by L via one of the following;
   a) a covalent bond, or
   b) a group for formula “-A¹-A²-A³-”;
      wherein the other terminal of A¹ and A³ is connected to L and D, respectively; and wherein A¹ and A³ is independently selected from: —CR¹R²—, —C(O)—, —C(O)NR¹—, —NR¹C(O)—, —NR¹—, —O—, —S—, —S(O)—, —S(O)₂—, —R¹C═CR²—, —C≡C—, —R¹C═CR²—R³C═CR⁴—, —R¹C═C═CR²—, —C(═NR¹)—,

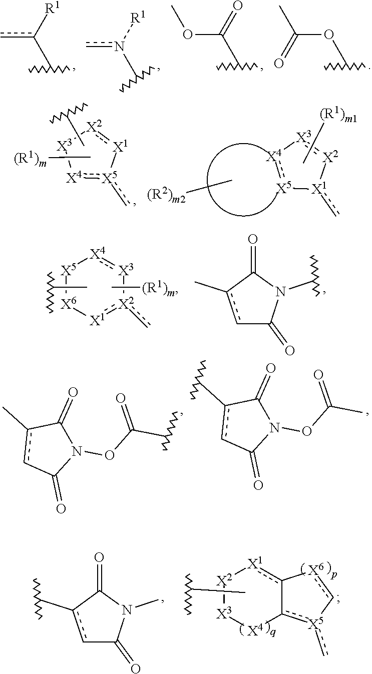

wherein
m, m1 and m2 are independently 0, 1, 2, 3, or 4; p is 1 or 2 and q is 0 or 1;
“ [symbol] ” indicates the point of attachment of A² to A¹ or A³;
“ [symbol] ” indicates single or double bond;
“ [symbol] ” indicates a 5 to 10 membered ring including atoms of the ring it is fused to, wherein the 5 to 10 membered ring is an aromatic, non-aromatic, or cycloalkyl ring or a heteroaromatic or heterocycloalkyl ring wherein the heteroaromatic or heterocycloalkyl ring comprise at least one heteroatom selected from N, O or S;
each of X¹, X², X³, X⁴, X⁵ and X⁶ is independently selected from C, CH, CH₂, N, NH, O or S; and
R¹, R², R³ and R⁴ are independently selected from hydrogen, C₁ to C₅ alkyl, C₂ to C₅ alkenyl, —C₂ to C₅ alkynyl, C₁ to C₅ alkylene, C₃ to C₁₀ cycloalkyl, C₂ to C₉ heterocycloalkyl, C₆ to C₁₀ aryl, C₂ to C₉ heteroaryl, —OR⁵, —NR⁵—SR⁵, halogen selected from fluorine, chlorine, bromine or iodine, and —C(O)OR⁵, wherein C₁ to C₅ alkyl, C₂ to C₅ alkenyl, —C₂ to C₅ alkynyl, C₁ to C₅ alkylene, C₃ to C₁₀ cycloalkyl, C₂ to C₉ heterocycloalkyl, C₆ to C₁₀ aryl, and C₂ to C₉ heteroaryl are unsubstituted or are each further substituted by R⁶, and
R⁵ and R⁶ are each independently hydrogen, C₁ to C₃ alkyl, OH or
halogen;
wherein A² is of formula: [symbol]
wherein
e, j, f, g and y are independently 0 or 1, provided e, f and y are not 0 simultaneously,
S is sulphur atom,
E, F, and Y is a spacer, and
G is an amino acid or a peptide chain comprising 2, 3, 4, 5, 6, 7, 8, 9, 10, 11 or 12 amino acids;
and wherein A¹ forms bond with E or S or F and A³ is connected to Y or G.

According to another aspect of the disclosure, there is provided a compound of Formula II:

L-D Formula II
or a pharmaceutically acceptable salt thereof, wherein
   L is a recombinant lectin derived from S. rolfsii;
   D is selected from a cytotoxic agent, therapeutic agent or a compound comprising cytotoxic agent or therapeutic agent.

According to yet another aspect of the present disclosure, there is provided a compound of Formula III:

[symbol]

or a pharmaceutically acceptable salt thereof, wherein L, F, G, Y, A³, D, n, j, f, g, and y are as defined herein before.

According to an aspect of the present disclosure, there is provided a compound of Formula IV:

[symbol]

or a pharmaceutically acceptable salt thereof, wherein L, A¹, F, G, Y, A³, D, j, f, g, and y are as defined herein.

According to the preceding aspects, the recombinant lectin derived from S. rolfsii, is selected from:
i. lectin protein having amino acid sequence of SEQ ID NO: 1, or
ii. lectin protein having at least 70% sequence identity to the SEQ ID NO: 1.

According to an aspect of the present disclosure, the recombinant lectin derived from S. rolfsii is selected from lectin having amino acid sequence of SEQ ID NO: 2, SEQ ID NO: 3, SEQ ID NO: 4, SEQ ID NO: 5, SEQ ID NO: 8; or the modified lectin proteins in WO2020044296, which is hereby incorporated by reference.

According to a particular aspect of the present disclosure, the recombinant lectin derived from S. rolfsii is selected from lectin having amino acid sequence of SEQ ID NO: 2 or SEQ ID NO: 5 or SEQ ID NO. 8.

In an aspect of the present disclosure, D is selected from a DNA binding agent, an anti-microtubule agent, an antimetabolite, an alkylating or alkylating-like antineoplastic agent, a topoisomerase I inhibitor, a topoisomerase II inhibitor, a kinase inhibitor, bortezomib, estramustine, Ixabepilone, everolimus, temsirolimus, neomycin, neamine, a cryptophycin, discodermolide, amanitin, or a pyrrolobenzodiazepine dimer, or a pharmaceutically acceptable salt, polymorph, or solvate of each thereof.

According to any preceding aspect of the present disclosure each spacer E, F and Y is independently selected from —R⁷—, —S—, —NH—, —R⁷—NH—R⁸—, —R⁸—NC(O)—R⁷—, —R—C(O)N—R⁷—, —C(O)—, —C(O)—R⁷—, —R⁷—C(O) R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, O—R⁷—, —R⁷—O—, —R—O—R⁷—, —C(O)O—, —OC(O)—R⁷—, —R⁷—OC(O)—, —R⁸—OC(O)—R⁷—, —R⁷—OC(O)O—, OC(O)O—R⁷—, —R⁷—OC(O)O—R⁸N, —NH—R⁷—NH—C(O)R⁸—, —(CH₂)S—NH—R⁷—, —R⁷—C(O)N(CH₂)₁₋₃O(CH₂)₁₋₃C(O)—, —R⁷—NC(O)(CH₂)₁₋₃O(CH₂)₁₋₃C(O)—, —Si(R⁷R₈)—, polyalkylene glycol optionally attached through oxygen to —R⁷—, —S—, —NH—, —R⁷—N—R⁸—, —R⁸—NC(O)—R⁷—, —R—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, —O—R⁷—, —R⁷—O—, —R—O—R⁷—, —C(O)O—, —OC(O)—R⁷—, —R⁷—OC(O) R⁸—OC(O)—R⁷—, —R⁷—OC(O)O—, —OC(O)O—R⁷—, —R⁷—OC(O)O—R⁸N, NH—R⁷—NH C(O)R⁷—, —(CH₂)S—NH—R⁷—, —R⁷—C(O)N(CH₂)₁₋₃O(CH₂)₁₋₃C(O)—, R⁷—NC(O)(CH₂)₁₋₃O(CH₂)₁₋₃C(O)—, —Si(R⁷R⁸)—;
wherein R⁷ and R⁸ are independently selected from hydrogen, C₁-C₁₀ alkylene, C₁-C₁₀ alkenylene, C₅ to C₁₀ saturated or unsaturated cycloalkylene, C₂-C₁₀ alkenylene-C₅ to C₁₀ saturated or unsaturated cycloalkylene, C₆ to C₁₀ arylene, —C₂-C₁₀ alkenylene-C₆ to C₁₀ arylene, heteroarylene ring comprising atleast one heteroatom selected from N, O and S, C₂-C₁₀ alkenylene-C₂ to C₉ heteroarylene; and wherein the C₁-C₁₀ alkylene, C₂-C₁₀ alkenylene, C₅ to C₁₀ saturated or unsaturated cycloalkylene, C₂-C₁₀ alkenylene-C₅ to C₁₀ saturated or unsaturated cycloalkylene, C₆ to C₁₀ arylene, C₂-C₁₀ alkenylene-C₆ to C₁₀ arylene, C₂ to C₉ heteroarylene and —C₂-C₁₀ alkenylene-C₂ to C₉ heteroarylene is optionally further substituted with —OH, —C(O), —SH, —NH₂, halogen, C₁ to C₅ alkyl, —NO₂, or —CN; wherein polyalkylene glycol is a homopolymer or copolymer of alkylene with each unit of alkylene having C₁ to C₈ carbon atoms and total number of alkylene units ranging from 1 to 20; and wherein when polyalkylene glycol is E the —CH₂— end is attached to A¹ and when polyalkylene glycol is F the —CH₂— end is attached to —R⁷—, —S—, —NH—, —R⁷—N—R⁸—, —R⁸—NC(O)—R⁷—, —R—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, —O—R⁷—, —R⁷—O—, —R⁸—O—R⁷—, —C(O)O—, —OC(O)—R⁷—, —R⁷—OC(O) R⁸—OC(O)—R⁷—, —R⁷—OC(O)O—, —OC(O)O—R⁷—, —R⁷—OC(O)O—R⁸—, —NH—R⁷—NH—C(O)R⁸— or —(CH₂)S—NH—R⁷—, —R⁷—C(O)N(CH₂)₁₋₃O(CH₂)₁₋₃C(O)— or —R⁷—NC(O)(CH₂)₁₋₃O(CH₂)₁₋₃C(O)—.

According to the preceding aspect of the present disclosure, Y and A³ independently or in combination comprise a self immolative groups.

In an aspect of the present disclosure, there is provided a method of treatment or prevention of cancer using compound of Formula I or Formula II or Formula III or Formula IV.

In another aspect of the present disclosure, there is provided a pharmaceutical composition comprising compound of Formula I or Formula II or Formula III or Formula IV.

In yet another aspect of the present disclosure, there is provided a compound of Formula I, Formula II, Formula III or Formula IV or a composition comprising compound of Formula I, Formula II, Formula III or Formula IV for use in the preparation of medicament for the treatment or prevention of cancer.

### Definitions

The term “alkyl” by itself or as part of another term refers to a substituted or unsubstituted straight chain or branched, saturated or unsaturated hydrocarbon having the indicated number of carbon atoms (e.g., “—C₁-C₈ alkyl” or “—C₁-C₁₀” alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl, -n-propyl, -n-butyl, -n-pentyl, -n-hexyl, -n-heptyl and -n-octyl; while branched —C₁-C₈ alkyls include, but are not limited to, -isopropyl, -sec-butyl, -isobutyl, -tert-butyl, -isopentyl, and -2-methylbutyl; unsaturated C₂-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexyl, 2-hexyl, -3-hexyl, -acetylenyl, -propynyl, -1-butynyl, -2-butynyl, -1-pentynyl, -2-pentynyl and -3-methyl-1 butynyl. In some embodiments, an alkyl group is unsubstituted. An alkyl group can be substituted with one or more groups. In some aspects, an alkyl group is saturated.

The terms “alkenyl” and “alkynyl” refer to straight and branched carbon chains having specified carbon atoms or from about 2 to about 20 carbon atoms (and all combinations and sub-combinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl. Examples of alkynyl groups include, but are not limited to, acetylenic, propargyl, acetylenyl, propynyl, -1-butynyl, -2-butynyl, -1-pentynyl, -2-pentynyl, and -3-methyl-1-butynyl.

Alkenyl and alkynyl groups, whether alone or as part of another group, can be optionally substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and CN, where each R′ is independently selected from H, —C₁-C₈ alkyl, —C₂-C₈ alkylenyl, C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH(R″), —N(R″)₂ and —CN, where each R″ is independently selected from H, —C₁-C₈ alkyl, —C₂-C₈ alkenyl, —C₂-C₈ alkynyl, or -aryl.

In further embodiments, the term “alkylene” refers to a saturated branched or straight chain hydrocarbon radical having specified number of carbon atoms or from about 1 to about 20 carbon atoms (and all combinations and sub-combinations of ranges and specific numbers of carbon atoms therein), with from about 1 to about 8 carbon atoms being preferred and having two monovalent radical centres derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and CN, where each R′ is independently selected from H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and CN, where each R″ is independently selected from H, —C₁-C₈ alkyl, —C₂-C₈ alkenyl, —C₂-C₈ alkynyl, or -aryl.

In further embodiments “aryl,” by itself or as part of another term, means a substituted or unsubstituted monovalent carbocyclic aromatic hydrocarbon radical of said number of carbon atoms or 6-20 carbon atoms, which is derived by the removal of one hydrogen atom from a single carbon atom of a parent aromatic ring system.

The term “arylene,” is an aryl group as defined above that has two substitution and can be in the ortho, meta, or para configurations based on relative position of two substitutions, wherein substitutions and 1 and 2 position is said to be ortho substitution, substitution at 1 and 3 position is meta and substitution at 1 and 4 position is para.

In further embodiments a “C₂-C₉ heterocycle,” by itself or as part of another term, refers to a monovalent substituted or unsubstituted aromatic or non-aromatic monocyclic or bicyclic ring system having from 2 to 9 carbon atoms (also referred to as ring members) and one to four heteroatom ring members independently selected from N, O or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

In further embodiments a “C₃-C₁₀ cycloalkyl or carbocycle,” by itself or as part of another term, is a 5-, 6-, 7-, 8-, 9- or 10-membered monovalent, substituted or unsubstituted, saturated or unsaturated non-aromatic monocyclic or bicyclic carbocyclic ring derived by the removal of one hydrogen atom from a ring atom of a parent ring system. Representative cycloalkyls include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

In further embodiments a carbocyclo by itself or as part of another term, refers to a carbocycle group defined above wherein another of the carbocycle groups' hydrogen atoms is replaced with a bond (i.e., it is divalent).

“Substituted alkyl” and “substituted aryl” mean alkyl and aryl, respectively, in which one or more hydrogen atoms are each independently replaced with a substituent. Typical substituents include, but are not limited to, —X, —R, —O₃₁, —OR, —SR, —S—, —NR₂, —NR₃, ═NR, —CX₃, —CN, —OCN, —SCN, —N═C═O, —NCS, —NO, —NO2, ═N2, —N3, —NRC(═O)R, —C(═O)R, —C(═O)NR2, —SO₃—, —SO₃H, δ(═O)₂R, —OS(═O)₂OR, —S(═O)₂NR, —S(═O)R, —OP(═O)(OR)₂, —P(═O)(OR)₂, —PO—₃, —PO₃H₂, —C(═O)R, —C(═O)X, —C(═S)R, —CO₂R, —CO₂—, —C(═S)OR—, —C(═O)SR—, —C(═S)SR—, —C(═O)NR₂, —C(═S)NR₂, or —C(═NR)NR₂, where each X is independently a halogen: —F, —Cl, —Br, or —I; and each R is independently —H, —C₁-C₂₀ alkyl, —C₆-C₂₀ aryl, —C₃-C₁₄ heterocycle, a protecting group or a prodrug moiety.

Alkylene, carbocycle, carbocyclo, arylene, heteroalkyl, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

The term “protein” as used herein refers to a polymer of amino acid residues.

The term “lectin” or “lectin protein” as used herein refers to a carbohydrate-binding protein of Sclerotium rolfsii (a soil borne pathogenic fungus of Indian origin), having National Center for Biotechnology Information (NCBI) Accession Number 2OFC_A.

The term “target cell” as used herein refers to a cell bearing receptors for lectins as defined aforementioned.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code and include the proteinogenic amino acids. Naturally occurring amino acids also include those modified after translation in cells. Synthetic amino acids include non-canonical amino acids such as selenocysteine and pyrrolysine. Typically, synthetic amino acids are not proteinogenic amino acids.

The term “recombinant” means a nucleic acid or a polypeptide has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule” is comprised of segments of DNA joined together by means of such molecular biological techniques. The term “recombinant protein” or “recombinant polypeptide” as used herein refers to a protein molecule which is expressed using a recombinant DNA technology.

The term “linker”, “linker unit”, or “link” as used herein refers to a chemical moiety comprising a covalent bond or a chain of atoms that covalently attaches a protein to a drug moiety. It is a bi-functional chemical moiety that covalently links the “L” and “D” moieties. The linker aids in maintaining the stability of the recombinant lectin drug conjugate throughout the plasma, by retaining its intactness till it reaches target site. The linkers are also labile and susceptible to the external stimuli of the tumour microenvironment, thereby cleaving/reducing the drug from the recombinant lectin protein drug conjugate, at the target site. The linkers are broadly classified into cleavable and non-cleavable linkers. Cleavable linker may be disulphide containing linkers that are cleavable through disulphide exchange or proteases; acid-labile linkers that are cleavable at acidic pH, and linkers that are cleavable by enzymes such as hydrolases, peptidases, esterases, and glucoronidases. The term “therapeutic agent” or therapeutically effective agent as used interchangeably here, means an agent administered to a subject for alleviating or curing one or more symptoms of the disease or disorder.

Cytotoxic agent refers to a substance that inhibits cells or inhibits the function of cells and/or causes destruction of cells. The term is intended to include chemotherapeutic agents, toxins such as small molecule toxins or enzymatically active toxins of bacterial, fungal, plant or animal origin, including synthetic analogues and derivatives thereof.

The term “conjugate” as used herein refers to the products of the present disclosure. For the purpose of present disclosure, the conjugate may comprise a cytotoxic agent attached to the recombinant lectin protein with the aid of a linker or it may a comprise cytotoxic agent directly attached to recombinant lectin protein. The structure of the conjugate and the bond between the cytotoxic agent and recombinant lectin protein is an aspect of the present disclosure and is as described herein.

A “spacer”, when present, extends the framework of the linker unit to provide more distance between the lectin protein and the cytotoxic agent. The spacer can also act to alter the physiochemical properties of the final products of the present disclosure depending on components of the spacer.

The term “compound comprising a cytotoxic agent” or “therapeutic agent” refers to a compound that comprises a linker or part of a linker along with a cytotoxic agent or therapeutic agent. Examples of compounds comprising a cytotoxic agent are given below.

The term “derivative (s) may refer to one or more of salts (e.g. pharmaceutically acceptable salts), esters, ethers, polymorphs, metabolites, pure form, particle size, isomers, mixtures of isomers, complexes, combinations of the compound(s).

The term “inhibit” or “inhibition of” means to a reduce by a measurable amount, or to prevent entirely.

The term “therapeutically effective amount” refers to an amount of a cytotoxic agent or therapeutic agent effective to treat a disease or disorder in a mammal. In the case of cancer, the therapeutically effective amount of the cytotoxic agent or therapeutic agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the cytotoxic agent may inhibit growth and/or kill existing cancer cells, it may be cytostatic and/or cytotoxic.

The term “cytotoxic activity” refers to a cell-killing, a cytostatic or an anti-proliferative effect of a compound or an intracellular metabolite of the compound.

Cytotoxic activity may be expressed as IC₅₀ value, which is the concentration in molar or mass per unit volume at which half the cells survive. The methods of determining IC₅₀ value are known to the person skilled in the art. For example, IC₅₀ value can be determined using standard MTS-PMS Cytotoxicity assay.

The terms “cancer” and “cancerous” refer to or describe the physiological condition or disorder in mammals that is typically characterized by unregulated cell growth.

A “tumor” comprises one or more cancerous cells.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

As used herein “preventing” or “prevention” refers to a reduction in the likelihood that a subject will develop the disease, relative to an untreated subject.

As used herein, the term “Stability unit” refers to a compound that promotes the stability of the conjugate, e.g., by increasing systemic retention of the Ligand when administered to a patient. A Stability unit can also increase the water solubility of the conjugate. An exemplary Stability unit is polyethylene glycol.

The term “excipient” as used herein refers to inactive or usually inert substances that are added to the formulation which do not affect the therapeutic action of the active ingredient, but serves as the vehicle or medium for the active ingredient. It may be used to provide a desired consistency, to improve stability, and/or to adjust osmolality of the composition. For the purpose of this disclosure excipients used are, but not limited to, protein stabilizing agents, buffers, polymers, solubilizers, Cryoprotectants, diluents or mixture thereof.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The recombinant lectin protein conjugate of the present disclosure may be provided in a pharmaceutically acceptable form, such as a liquid (e.g in an aqueous solution or suspension, or as an oil based solution or suspension.), a solid (e.g a capsule or tablet), a lyophilized powder, a spray, cream, lotion or gel, vesicular drug delivery systems such as, but not limited to, bilosomes, liposomes, niosomes, transferosome, ethosomes, sphingosomes, pharmacosomes, multilamellar vesicles, microsphere and the like.

The terms “homology” or “homologous” as used herein refer to two or more referenced entities that share at least partial identity over a given region or portion. Areas, regions or domains of homology or identity refer to a portion of two or more referenced entities that share homology or are the same. Thus, where two sequences are identical over one or more sequence regions, they share identity in these regions. Substantial homology refers to a molecule that is structurally or functionally conserved such that it has or is predicted to have at least partial structure or function of one or more of the structures or functions (e.g., a biological function or activity) of the reference molecule, or a relevant/corresponding region or portion of the reference molecule to which it shares homology.

In one embodiment, the percentage “homology” between two sequences is determined using the BLASTP algorithm with default parameters (Altschul et al. Nucleic Acids Res. 1997 Sep. 1; 25(17):3389-402). In particular, the BLAST algorithm can be accessed on the internet using the URL: https://blast.ncbi.nlm.nih.gov/Blast.cgi. In an alternative embodiment, for global sequence alignments, percentage homology between two sequences is determined using the EMBOSS Needle algorithm using default parameters. In particular, the EMBOSS Needle algorithm can be accessed on the internet using the URL: https://www.ebi.ac.uk/Tools/psa/emboss_needle/.

The term “sequence identity” as used herein refers to the occurrence of exactly the same nucleotide or amino acid in the same position in aligned sequences. Unless otherwise indicated, the term “homology” is used interchangeably with the term “sequence identity” or “identity” in the present specification.

The terms “Polyalkylene glycol” or “polyoxyalkylene glycol” are used interchangeably and mean polymers of alkylene oxide. The polymer may be a homopolymer comprising same alkylene oxide, for example polymer comprising all units of ethylene oxide is homopolymer know as polyethylene glycol. The polymer may also be a copolymer comprising different alkylene oxide, for example a polymer comprising three units of ethylene oxide and three units of propylene oxide is known as copolymer. Polyalkylene glycol can be prepared by the processes known to the skilled person or can be procured commercially.

As used herein, the term “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, humans.

Polymorphism is the occurrence of different crystalline forms of a single compound. The term “polymorph” as used herein mean the solids sharing the same molecular formula, yet each having distinct physical properties such as different solubility profiles and/or different melting point temperatures. The polymorph of a compound can be distinguished by X-ray diffraction spectroscopy or by other methods such as, infrared spectrometry.

The term “self-immolative” can also be referred to as self-eliminating group and are chemical linkers that undergo a sequence of self-immolative reactions typically upon cleavage of one or more trigger units. Such self immolative chemical linkers are preferably based on a multifunctional unit which can be linked to both the chemical moiety and to two or more trigger units or other chemical linkers and can further be subjected to electronic cascade self-elimination. A self-immolative group can be selected as per understanding and knowledge of the skilled person or from the references including US patent application 20060269480 or “Self-immolative linkers in polymeric delivery systems” (Blencowe et al., Polymer Chemistry 2, 773-790, 2011).

When one or more molecules of solvent are trapped in the crystal of the compound, the compound is said to be solvated. The term ‘solvate’ may be defined as a compound formed by solvation. The solvates may be prepared by using solvents known to the skilled person. Non-limiting examples of solvents are water, alcohols such as methanol, ethanol, n-propanol, isopropanol, n-butanol, isobutanol, and t-butanol, glycols or polyalkylene glycols (e.g., polyethylene glycol, polypropylene glycol), acetic acid, formic acid, acetone, dichloromethane and/or mixture thereof. The best-known and preferred solvent is typically water, and solvate compounds formed by solvation with water are termed hydrates.

Sequence Representation:
SEQ ID NO: 1:
represents the native S. rolfsii lectin amino
acid sequence (reported as SEQ ID NO: 1 in
WO2010/095143), has the following sequence:
TYKITVRVYQTNPNAFFHPVEKTVWKYANGGTWTITDDQHVLTMG
GSGTSGTLRFHADNGESFTATFGVHNYKRWCDIVINLAADETGMV
INQQYYSQKNREEARERQLSNYEVKNAKGRNFEIVYTEAEGNDLH
ANLIIG
SEQ ID NO: 2:
represents a variant of the S. rolfsii lectin
amino acid sequence (reported as Rec-2 in
WO2010/095143) has the following sequence:
TYKITVRVYQTNPDAFFHPVEKTVWKYANGGTWTITDDQHVLTMG
GSGTSGTLRFHADNGESFTATFGVHNYKRWCDIVTNLAADETGMV
INQQYYSQKNREEARERQLSNYQVKNAKGRNFQIVYTEAEGNDLH
ANLIIG
SEQ ID NO: 3:
represents a variant of the S. rolfsii lectin
amino acid sequence (reported as Rec-3 in
WO 2010/095143) has the following sequence:
VYKITVRVYQTNPDAFFHPVEKTVWKYANGGTWSITDDQHVLTMG
GSGTSGTLRFHADNGESFTATFGVHNYKRWCDIVTNLAADETGMV
INQQYYSQKNREEARERQLSNYQVKNAKGRNFQIVYTEAEGNDLH
ANLIIG
SEQ ID NO: 4:
represents a variant of the S. rolfsii lectin
amino acid sequence (reported in WO 2014/203261)
has the following sequence:
VYKITVRVYQTNPDAFFHPVEKTVWKYADGGTWSITDDQHVLTMG
GSGTSGTLRFHADNGESFTATFGVHDYKRWCDIVTDLAADETGMV
INQEYYSEKDREEARERQNSNYEVKDAKGRNFEIVYTEAEGNDLH
ADLIIG
SEQ ID NO: 5:
represents a variant of the S. rolfsii lectin
amino acid sequence has
the following sequence:
SYKITVRVYQTNPDAFFHPVEKTVWKYANGGTWTITDDQHVLTMG
GSGTSGTLRFHADNGESFTATFGVHNYKRWGDIVTNLAADETGMV
INQQYYSQKNREEARERQLSNYQVKNAKGRNFQIVYTEAEGNDLH
ANLIIGC
SEQ ID NO: 6:
represents a Nucleotide sequence encoding a
protein of SEQ ID NO. 2
ATGACCTATAAAATTACCGTGCGCGTGTATCAGACCAACCCGGAT
GCCTTTTTCCATCCGGTGGAAAAAACCGTGTGGAAATATGCGAAT
GGCGGTACCTGGACGATTACGGATGATCAGCATGTGCTGACGATG
GGTGGTAGCGGTACCAGCGGCACCCTGCGTTTTCACGCAGATAAT
GGCGAAAGCTTCACCGCCACCTTTGGTGTGCATAATTATAAACGC
TGGTGTGATATTGTGACCAACCTGGCAGCGGATGAAACCGGCATG
GTTATTAATCAGCAGTATTATAGTCAGAAAAACCGCGAAGAAGCG
CGTGAACGCCAGCTGAGTAACTATCAGGTGAAAAATGCGAAAGGC
CGTAACTTCCAGATTGTTTATACCGAAGCGGAAGGCAATGATCTG
CATGCGAACCTGATTATCGGC
SEQ ID NO: 7:
represents a Nucleotide sequence encoding
SEQ ID NO. 5
AGCTATAAAATTACCGTGCGCGTGTATCAGACCAACCCGGATGCC
TTTTTCCATCCGGTGGAAAAAACCGTGTGGAAATATGCGAATGGC
GGTACCTGGACGATTACGGATGATCAGCATGTGCTGACGATGGGT
GGTAGCGGTACCAGCGGCACCCTGCGTTTTCACGCAGATAATGGC
GAAAGCTTCACCGCCACCTTTGGTGTGCATAATTATAAACGCTGG
GGCGATATTGTGACCAACCTGGCAGCGGATGAAACCGGCATGGTT
ATTAATCAGCAGTATTATAGTCAGAAAAACCGCGAAGAAGCGCGT
GAACGCCAGCTGAGTAACTATCAGGTGAAAAATGCGAAAGGCCGT
AACTTCCAGATTGTTTATACCGAAGCGGAAGGCAATGATCTGCAT
GCGAACCTGATTATCGGCTGC
SEQ ID NO. 8 represents a variant of the
S. rolfsii lectin amino acid sequence
SYKITVRVYQTNPDAFFHPVEKTVWKYANGGTWTITDDQHVLTMG
GSGTSGTLRFHADNGESFTATFGVHNYKRWGDIVTNLAADETGMV
INQQYYSQKNREEARERQLSNYQVKNAKGRNFQIVYTEAEGNDLH
ANLIIGSC
SEQ ID NO. 9 represents a Nucleotide sequence
encoding a protein of SEQ ID NO. 8
AGCTATAAAATTACCGTGCGCGTGTATCAGACCAACCCGGATGCC
TTTTTCCATCCGGTGGAAAAAACCGTGTGGAAATATGCGAATGGC
GGTACCTGGACGATTACGGATGATCAGCATGTGCTGACGATGGGT
GGTAGCGGTACCAGCGGCACCCTGCGTTTTCACGCAGATAATGGC
GAAAGCTTCACCGCCACCTTTGGTGTGCATAATTATAAACGCTGG
GGCGATATTGTGACCAACCTGGCAGCGGATGAAACCGGCATGGTT
ATTAATCAGCAGTATTATAGTCAGAAAAACCGCGAAGAAGCGCGT
GAACGCCAGCTGAGTAACTATCAGGTGAAAAATGCGAAAGGCCGT
AACTTCCAGATTGTTTATACCGAAGCGGAAGGCAATGATCTGCAT
GCGAACCTGATTATCGGCAGCTGC

### DETAILED DESCRIPTION OF THE DRAWINGS

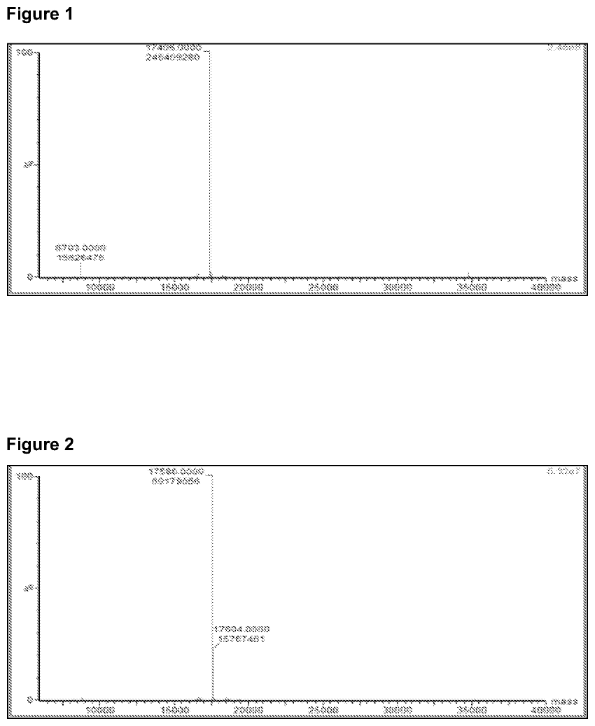

FIG. 1: Represents reverse phase Mass spectrum for conjugate of Formula 16. The peak 17406 confirms the formation of conjugate of Formula 16 which is also the mass of the final product Formula 16.

FIG. 2: Represents reverse phase Mass spectrum for conjugate of Formula 18. The peak 17586 confirms the formation of conjugate of Formula 18 which is also the mass of the final product Formula 18.

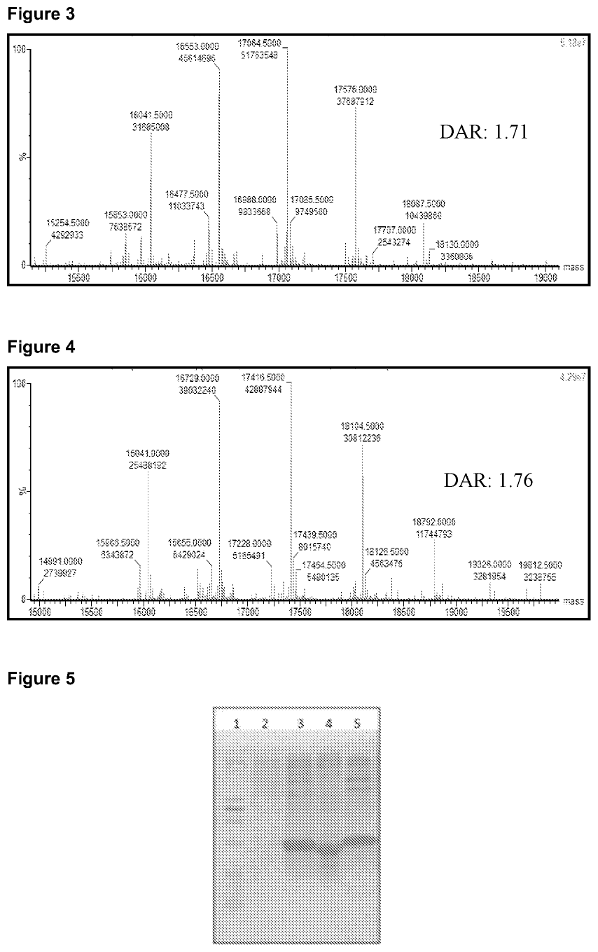

FIG. 3: Represents LC/MS of Formula C with x=8. The peak at 16041.5000 is related to L alone, whereas peaks at 16553, 17064.5 and 17576 show the formation of Formula C with DAR 1, 2 and 3, respectively. The average DAR being 1.71.

FIG. 4: Represents LC/MS of Formula C with x=12. The peaks at 16729, 17416.5 and 18104.5 show the formation of Formula C having varying DAR values 1, 2 and 3, respectively with average DAR of 1.76

FIG. 5: Expression and Solubility analysis of recombinant lectin of SEQ ID NO: 5 in production medium. The figure represents SDS-PAGE analysis of SEQ ID NO. 5 expression at different times as given below:

| Lane | Sample | Band % |
|---|---|---|
| 1 | Standard Protein Marker | NA |
| 2 | Seq ID NO: 5 0 hr | — |
| 3 | Seq ID NO: 5 overnight | 56 |
| 4 | Seq ID NO: 5 overnight pellet | 49.6 |
| 5 | Seq ID NO: 5 supernatant | 47.2 |

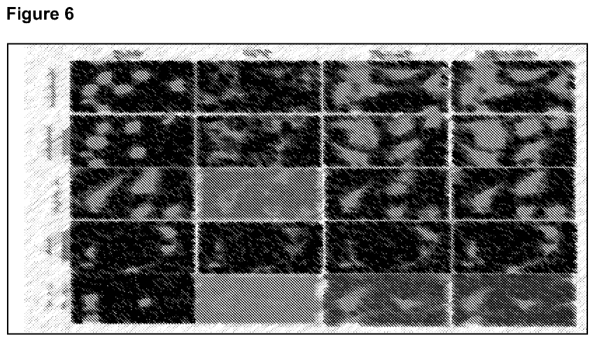

FIG. 6: Colocalization of Recombinant Lectin having amino acid sequence of SEQ ID NO:2 (Recombinant protein) in different organelles such as mitochondria, golgi bodies, plasma membrane, nuclear envelope, endoplasmic reticulum in 1 cell line.

### DETAILED DESCRIPTION

According to a first aspect of the present disclosure, there is provided a compound of Formula I:

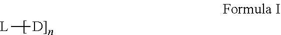

or a pharmaceutically acceptable salt thereof.

According to an aspect, L is a recombinant lectin derived from Sclerotium rolfsii (S. rolfsii) lectin. S. rolfsii is a soilborne phytopathogenic fungus and Sclerotium rolfsii lectin (SRL) is a lectin isolated from S. rolfsii. The native SRL has an amino acid sequence of SEQ ID NO: 1.

In an aspect, L is a recombinant lectin having amino acid sequence of SEQ ID NO: 1 or a recombinant lectin having at least 70% sequence identity to SEQ ID NO: 1. In some embodiments the recombinant lectin protein comprises an amino acid sequence having at least 70%, 80%, 90%, 95%, 96%, 97%, 98% or 99% homology to SEQ ID NO: 1.

In an embodiment of the present disclosure, recombinant lectin is a variant of Sclerotium rolfsii Lectin (SRL) represented by SEQ ID NO. 1. In a particular embodiment, the said recombinant lectin retains binding specificity or affinity of the Sclerotium rolfsii Lectin (SRL) represented by SEQ ID NO. 1. The said binding specificity is envisaged to be toward one or more antigens selected from Thomsen-Friedenreich (Galβ1-3GalNAca-O-Ser/Thr, TF or T) antigen, O-GalNAc Core1 (T antigen), Core2, ‘α2,3/6-sialyl Core1’ (Sialyl-T antigen), ‘α2,6/6-sialyl Core2’ and its modified forms such as and altered expression of branched and fucosylated N- and O-glycans, including changes in Lewis antigens (SLex and SLea) which are expressed in more than 90% of human cancers. In addition to alterations in core glycans, each of these carbohydrates can be further modified to generate unique terminal glycan motifs that may also undergo specific changes following neoplastic transformation. For example, highly fucosylated glycans, such as Lewis antigens [Lewisa/b (Lea/b) and Lewisx/y (Lex/y)], can become enriched on the cell surface following neoplastic transformation. Similarly, sialylation, a common terminal glycan modification, can also undergo significant changes during neoplastic progression.

In a more particular embodiment of the present disclosure, variants or modified forms of TF (or T) antigen includes [3OSO3]Galβ1-3GalNAcα; Neu5Acα2-3Galβ1-3 [6OSO3]GalNAcα; Neu5Acα2-3Galβ1-3GalNAcα; GlcNAcβ1-3Galβ1-3GalNAcα; Galβ1-3(Galβ1-4GlcNAcβ1-6) GalNAcα; Galβ1-3(Neu5Acα2-3Galβ1-4GlcNAβ1-6)GalNAc; Galβ1-3(GlcNAcβ1-6)GalNAcα; Galβ1-3(Neu5Acα2-6)GalNAcα; Neu5Acα2-3Galβ1-3(Neu5Acα2-6)GalNAcα; Galβ1-4GlcNAcβ1-6(Galβ1-3) GalNAcα; Galβ1-3GalNAcα (TF antigen); Neu5Acα2-6 (Galβ11-3)GalNAcα; Galβ1-3(Galβ1-4GlcNAcβ1-6)GalNAc; Galβ1-3(GlcNAcβ1-6)GalNAc; GlcNAcβ1-6 (Galβ1-3) GalNAcα; Galβ1-3 (Neu5Acβ2-6)GalNAcα; Fucα1-2Galβ1-3GalNAcα; Neu5Acβ2-6 (Galβ1-3)GalNAcα; GlcNAcβ1-2Galβ1-3GalNAcα.

WO 2010/095143 discloses recombinant lectin variants Rec-2 and Rec-3, which are derived from the native SRL sequence by the substitution of 3 or 5 amino acids respectively. The crystal structure of these variants has been reported (Peppa et al., Molecules. 2015 Jun. 12; 20(6):10848-65). Rec-2 and Rec-3 is mentioned as SEQ ID NO: 2 and SEQ ID NO: 3, respectively hereinafter. WO 2014/203261 discloses a recombinant lectin variant (mentioned as SEQ ID NO: 4 hereinafter) derived from the native SRL sequence by the substitution of 12 amino acids. WO 2020/044296 discloses several variants of SRL with at least one amino acid modification selected from one or more of the following:
a) at least one amino acid modification in a carbohydrate binding site selected from position 27, 28, 47, 48, 70, 71, 72, 77, 78, 80, 101, 105, 112 or 114;
b) at least one amino acid modification in the N-terminus;
c) at least one amino acid modification at position 76; or
d) at least one amino acid modification at position 44 and 89.

WO 2020/044296 further discloses SRL variants with amino acid modifications, wherein the modification refers to addition of at least 1, 2, 3, 4 or 5 amino acids at a particular position in an amino acid sequence.

WO 2020/044296 is incorporated in the present application by way of reference in its entirety. Amino acid sequences from SEQ ID NO: ULLB-0005/001 to SEQ ID NO: ULLB-0005/047 of WO 2020/044296 are embodied as an aspect of the present disclosure and are not repeated for the sake of brevity. In a further embodiment, SEQ ID NO: 5 disclosed in present disclosure is a variant of SEQ ID NO: 1 with modification at 1^{st} position T1S; 76^{th} position C76G and with addition of cysteine at C terminal end i.e. 142nd position.

In a further embodiment, SEQ ID NO. 8 disclosed in the present disclosure is a variant of Seq ID No. 5 with an insertion of amino acid Serine at 142 position between Glycine (141 position) and Cysteine (C terminal end).

SEQ ID NO: 2, 3, 4, 5 and 8 have a homology of 97.9%, 96.5%, 91.5%, 96.45% and 96.45% to SEQ ID NO: 1 respectively (as determined using EMBOSS Needle).

According to an aspect the lectin proteins of the present disclosure are prepared by recombinant technology. The term “recombinant” means a nucleic acid or a polypeptide has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule” is comprised of segments of DNA joined together by means of such molecular biological techniques. The term “recombinant protein” or “recombinant polypeptide” as used herein refers to a protein molecule which is expressed using a recombinant DNA technology.

In some embodiments the lectin protein of the present disclosure may be prepared by the processes disclosed in WO 2010/095143, WO 2014/203261, WO 2020/044296 or applicant's previously filed process patent WO2020074977 or any other method known to the skilled person.

The term “recombinant protein” is intended here to cover any pharmaceutically acceptable salt, solvate, hydrate, prodrug, or any other compound which, is capable of providing (directly or indirectly) the compound as described herein. The preparation of salts, solvates, hydrates, and prodrugs can be carried out by methods known in the art. According to a specific aspect of the present disclosure, L is a recombinant protein selected from SEQ ID NO: 2, SEQ ID NO: 3, SEQ ID NO: 4, SEQ ID NO: 5 and SEQ ID NO: 8. According to a very particular aspect, L is a recombinant protein selected from SEQ ID NO: 2 or SEQ ID NO: 5 or SEQ ID NO: 8.

In an aspect of the present disclosure, D in Formula I is a cytotoxic agent, therapeutic agent, diagnostic agent or a compound comprising cytotoxic agent or therapeutic agent. According to an aspect, D may be selected from a DNA binding agent, an anti-microtubule agent, an antimetabolite, an alkylating or alkylating-like antineoplastic agent, a topoisomerase I inhibitor, a topoisomerase II inhibitor, a kinase inhibitor, bortezomib, estramustine, Ixabepilone, everolimus, temsirolimus, neomycin, neamine, a cryptophycin, discodermolide, amanitin, or a pyrrolobenzodiazepine dimer, or pharmaceutical acceptable salt, polymorph or solvate thereof.

According to an aspect, D is selected from the DNA binding agent such as bleomycin, netropsin or distamycin or their analogues such as lexitropsins, an enediyne, a mitomycin, a duocarmycin. According to an embodiment the bleomycin may be selected from non-limiting examples of bleomycin A₂, bleomycin B₂, liblomycin, bleomycin substituted with butylamino-3-propylamino-3-propylamine (BAPP), Tallysomycin S10b (TLM Slob), or mixtures thereof, an enediyne for example, may be selected from neocarzinostatin, C-1027, golfomycin A, calicheamicins such as calicheamicin gamma 1 or calicheamicin omega 1; esperamicins such as esperamicin A1, esperamicin Alb, esperamicin A2, esperamicin A3, esperamicin A4, esperamicin B1, esperamicin B2, and esperamicin X; dynemicin or dynemicin A or lidamycin; the mitomycin may be selected from mitomycin A, mitomycin B or mitomycin C; non-limiting examples of duocarmycin are duocarmycin A, duocarmycin B1, duocarmycin B2, duocarmycin C1, duocarmycin C2, duocarmycin D, duocarmycin SA or CC-1065 and its synthetic analogues such as adozelesin, carzelesin or bizelesin; other non-limiting examples of DNA binding agents are dactinomycin, peptide such as echinomycin and other such agents listed in ‘anticancer drugs acting via radical species’ (by C. A. Lopez et al. medicinal chemistry of anticancer drugs, chapter 4, pages 133-195, December 2015) or ionic liquids listed in ‘synthesis, characterization, dna binding, anticancer, and molecular docking studies of novel imidazolium-based ionic liquids with fluorinated phenylacetamide tethers’ (by N Rezki et al. ACS Omega, volume 5, pages 4807-4815, 2020).

In an embodiment D may be selected from anti-microtubule agent such as an auristatin, a maytansine or its derivatives maytansinoid. These classes of compounds are well known payloads used in the synthesis of antibody-drug conjugates. Non-limiting examples of such class of compounds are dolastatin or its analogues include dolastatin 10, monomethyl auristatin E (MMAE), monomethyl auristatin F (MMAF) or PF-063801010 or any other auristatin derivatives listed in ‘antibody-drug conjugate payloads, study of auristatin derivatives; (by M. Akaiwa et el. Chemical pharma bulletin, volume 68, page 201-211, 2020), maytansinoids such as DM1, DM4 or DM 21. Other non-limiting examples of anti-microtubule agents may be a vinca alkaloid such as vinblastine, vincristine, vindesine or vinorelbine; colchicine or its derivatives, a taxane such as paclitaxel, docetaxel or cabazitaxel, a combretastatin such as combretastatin A-4, combretastatin A-4 phosphate (CA-4P), an epothilone such as epothilones A, epothilones B, epothilones C or epothilones D, eribulin, plinabulin, demecolcine, nocodazole, eleutherobin, rhizoxin, ethidium bromide, or verubulin. In an embodiment D is selected from non-limiting examples of antimetabolite such as capecitabine, hydroxyurea, gemcitabine, methotrexate, mercaptopurine, capecitabine, hydroxyurea, gemcitabine, methotrexate, mercaptopurine, cladribine, pralatrexate, raltitrexed fludarabine, pemetrexed, thioguanine, floxuridine, cytarabine, azacitidine, decitabine, clofarabine, nelarabine, 5-fluorouracil or carmofur; or topoisomerase II inhibitor such as daunorubicin, doxorubicin or its derivatives such as morpholino-doxorubicin, cyanomorpholino-doxorubicin, 2-pyrrolino-doxorubicin and deoxydoxorubicin, epirubicin, idarubicin, mitoxantrone, valrubicin, etoposide, teniposide or anthraquinones; or topoisomerase I inhibitor such as Irinotecan, SN-38, govitecan, camptothecin, exatecan (Dxd 1), deruxtecan, topotecan; or alkylating or alkylating-like antineoplastic agent such as a nitrogen mustard such as mustine, cyclophosphamide, chlorambucil, uramustine, melphalan, and bendamustine; ethylenimine derivatives such as aziridine, dimethylenimine, ethyleneimine; or methylenamine derivative or an alkyl sulfonate such as busulfan, improsulfan or piposulfan or a nitrosourea such as arabinopyranosyl-N-methyl-N-nitrosourea, carmustine, chlorozotocin, ethylnitrosourea (ENU), fotemustine, lomustine, nimustine, N-nitroso-N-methylurea (NMU), ranimustine, semustine, streptozocin (streptozotocin); platinum-containing antineoplastic agent such as cisplatin, carboplatin, oxaliplatin, nedaplatin, and lobaplatin; kinase inhibitor such as sorafenib, crizotinib, nilotinib, gefitinib, regorafenib, afatinib, lapatinib, vatalanib, aflibercept, axitinib, ponatinib, sunitinib, pazopanib, bosutinib, dasatinib, erlotinib, imatinib, ruxolitinib, lestaurtinib, AC-430, AC-480, AMG-458, or vandetanib; Pyrrolobenzodiazepine dimer such as tesirine, SG2202, SG3199, talirine, SG2000, SJG-136, SG2285, or SG3376 or pharmaceutical acceptable salt, polymorph or, solvate thereof.

In an embodiment, D may further be selected from non-limiting examples of bortezomib, sutent, letrozole, fulvestrant, leucovorin sirolimus, lonafarnib, a camptothecin (including the synthetic analog topotecan); cryptophycins (particularly cryptophycin 1 and cryptophycin 8); eleutherobin; pancratistatin; spongistatin; chlorambucil, chlornaphazine, chlorophosphamide, estramustine, ifosfamide, mechlorethamine, melphalan, prednimustine, trofosfamide, chromophore (chromoprotein enediyne antibiotic chromophores), aclacinomysins, authramycin, azaserine, cactinomycin, carabicin, carminomycin, carzinophilin, chromomycinis, marcellomycin, nogalamycin, olivomycins, peplomycin, porfiromycin, puromycin, quelamycin, streptonigrin, tubercidin, ubenimex, zinostatin; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as, dromostanolone propionate, epitiostanol, mepitiostane, testolactone; anti-adrenals such as aminoglutethimide, mitotane, trilostane; aceglatone; aldophosphamide glycoside; eniluracil; amsacrine; bestrabucil; bisantrene; demecolcine; diaziquone; an epothilone; etoglucid; gallium nitrate; hydroxyurea; razoxane; sizofiran; spirogermanium; triaziquone; trichothecenes (especially T-2 toxin, verracurin A, roridin A and anguidine); mitobronitol; mitolactol; pipobroman; arabinoside (“Ara-C”) 6-thioguanine; difluoromethylornithine (DMFO); retinoids such as retinoic acid; dacarbazine. D may be used as such or as its salt or derivatives. D may be its most active isomer including stereoisomer or any other isomer having moderate to high cytotoxicity. D may be also selected from toxins (ricin, Abrin, Pesudomonas Exotoxin PE) or peptides (WT1, GV1001, PAP-114-128, Erbb-2, TCP-1, ANP, F56) or protacs (BETd-260, MZ1ARV-771) having cytotoxic effect that are well known to the skilled person.

In an embodiment D may be selected from the compounds comprising a cytotoxic agent, therapeutic agent or a diagnostic agent. Such compounds may comprise a linker or part of the linker along with cytotoxic agent or therapeutic agent. Such linker or part of the linker would aid in attachment of D to L. Some of the non-limiting examples of such compounds comprising cytotoxic agents or therapeutic agent are given below, wherein M is maleimide, C is caproyl group, V, C, A, G, F are amino acids valine, citrulline, alanine, glycine, phenylalanine and PAB is para-aminobenzyloxy carbonyl. N-succinimidyl-4-thio butanoate-DM4 is formed when DM4 is reacted with a linker such as N-Succinimidyl-4-(2-pyridyldithio) Butanoate (SPDB), N-succinimidyl 4-(maleimidomethyl)cyclohexanecarboxylate (SMCC).

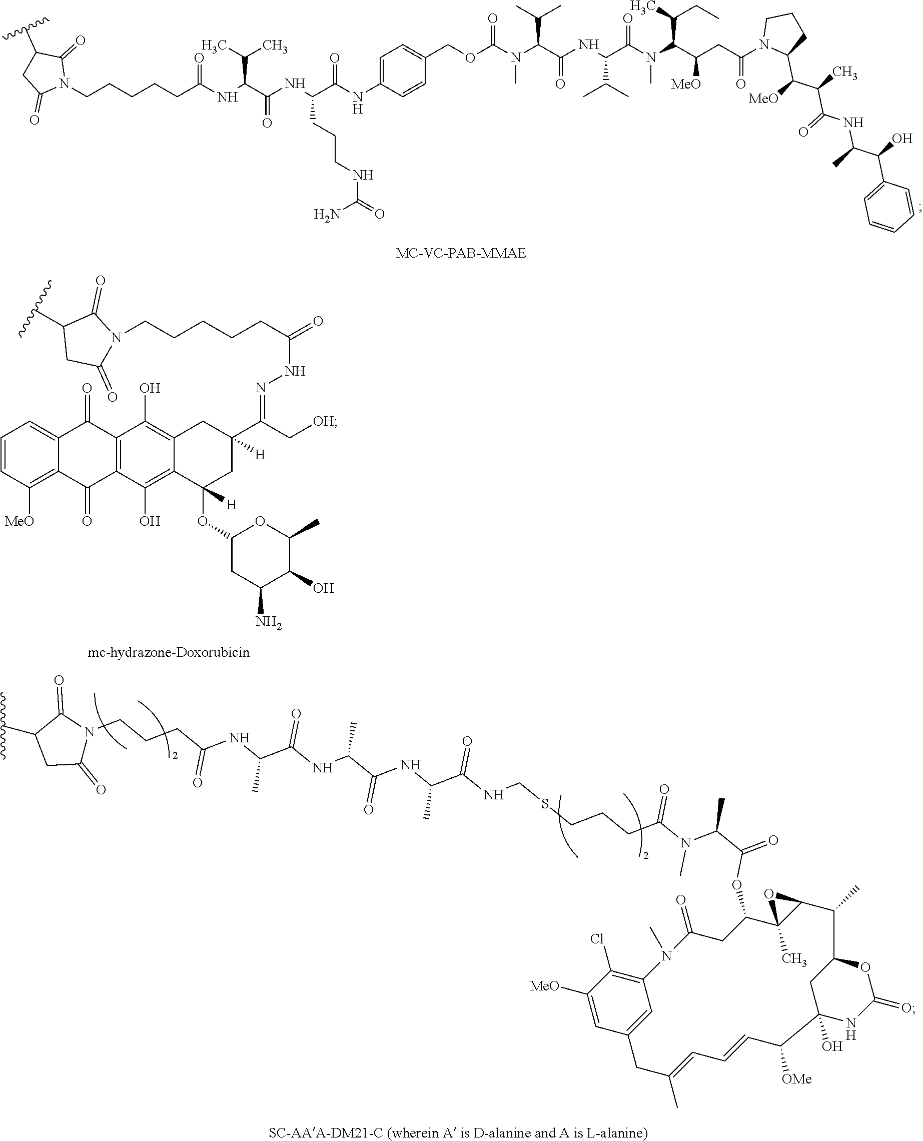

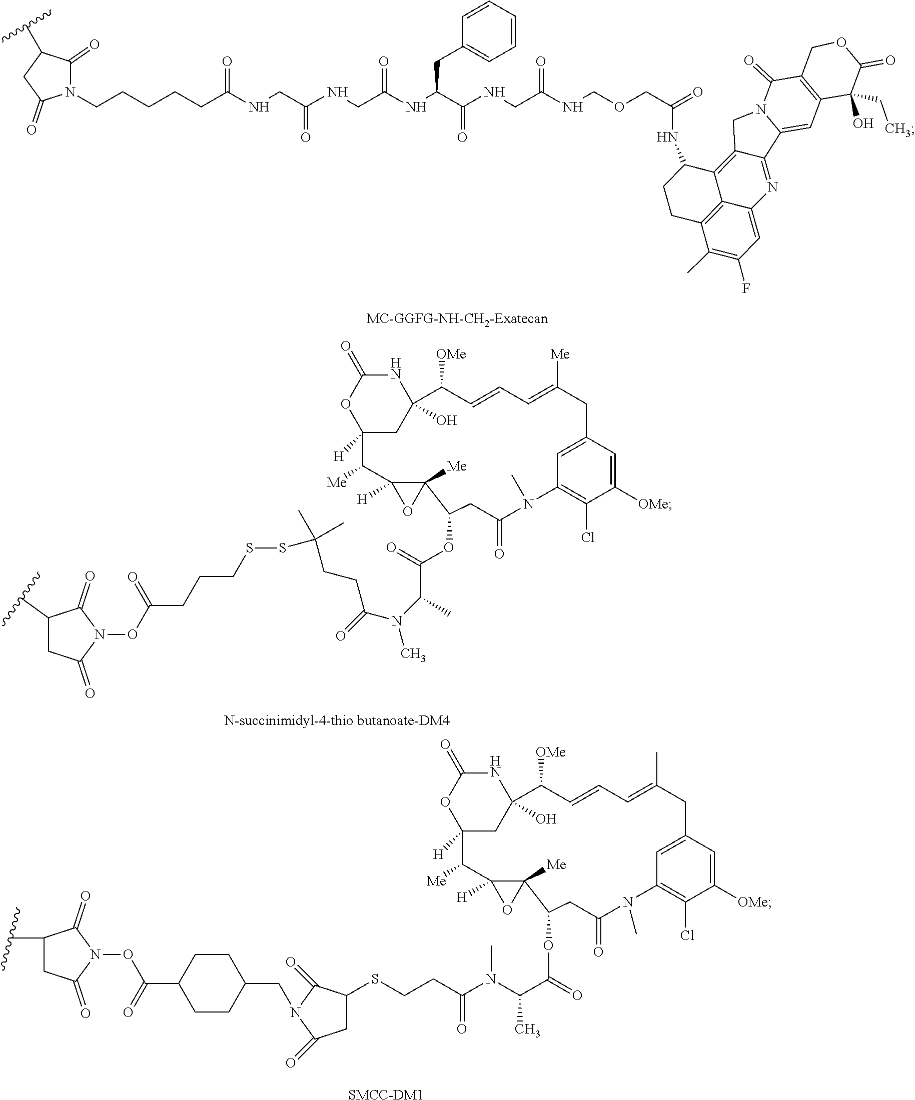

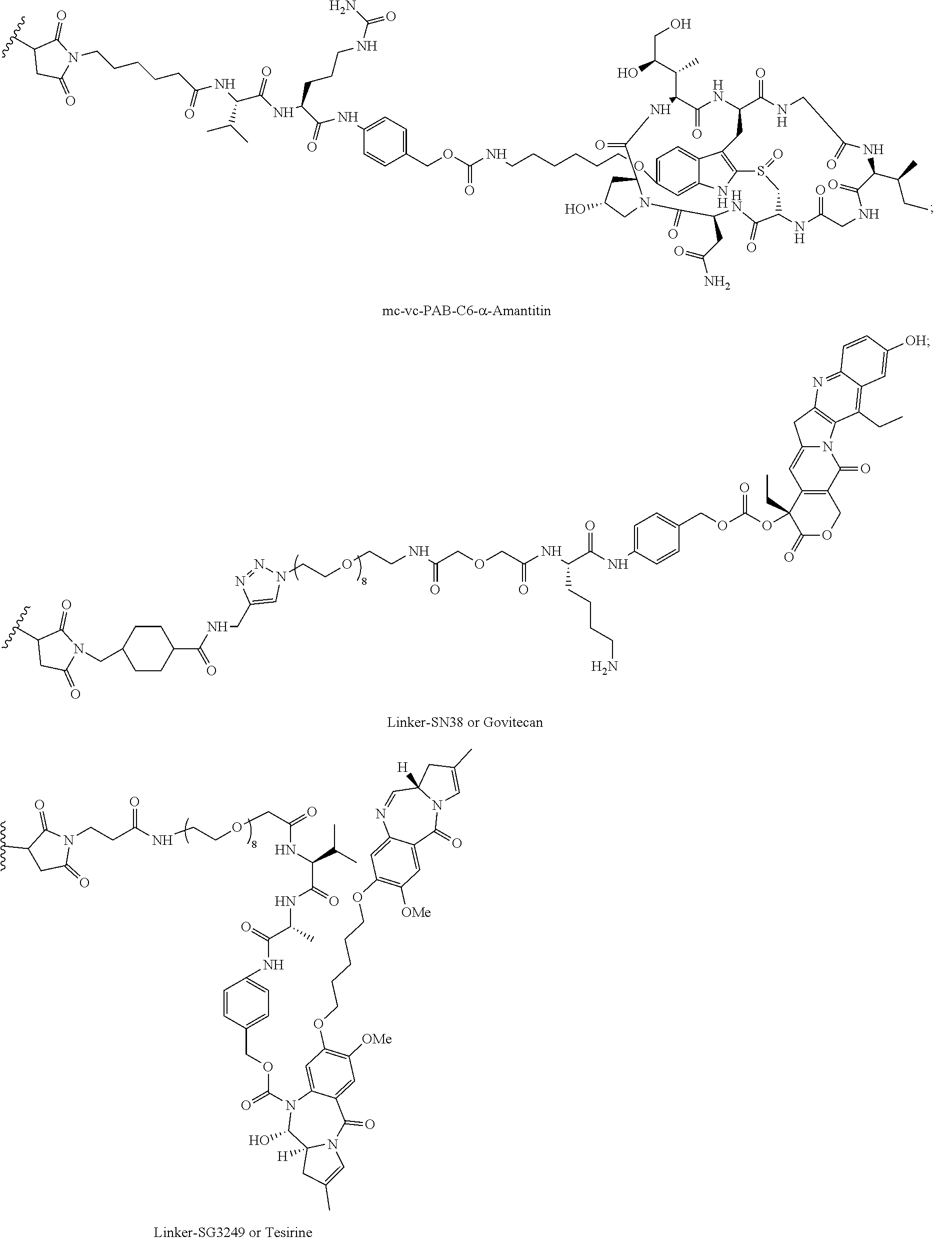

In a particular embodiment the cytotoxic agents are selected from but not limited to MMAE (monomethyl Auristatin E), Doxorubicin, DM1, DM4, DM21, alpha Amanitin, DXd1, SG3249, SN38, linked to linker selected from but not limited to mc-vc-PAB (melamide Caproic-valine citrulline-Para aminobenzoate), SMCC (Succinimidyl 4-(N-maleimidomethyl)cyclohexane-1-carboxylate, SPDB (N-Succinimidyl-4-(2-pyridyldithio) Butanoate), mc-vc-PAB-C₆(melamide Caproic-valine-citrulline-Para aminobenzoate-C₆), mc-hydrazone.

According to an aspect of the present disclosure the compound D, may be prepared by any process known in the art. For the purpose of the present disclosure D was procured commercially.

The lectin protein, L may be linked to D or to A¹ or to A² in the absence of A¹ via its free amino moiety (—NH— or —NH₂ group) of amino acids such as lysine, arginine or its free thiol moiety (—SH group) of amino acid such as cysteine or its free acid (—COOH) group of amino acid such as aspartic acid or its free OH group present in amino acids such as serine. It will be appreciated by the skilled person that all these groups on lectin may form a bond with reactive counterparts on D or A¹ or A² in the absence of A¹, thus resulting in more than one unit of D or -A¹-A²-A³-D linked to one unit of L.

In an aspect of the present disclosure, one unit of lectin protein, L may be linked to at least one unit of D. In an embodiment one unit of lectin protein may be linked to more than one unit of D. In another embodiment one unit of lectin protein may be linked to at least 2, 3, 4, 5, 6, 7, 8, or 9 units of a D. In antibody drug conjugates such ratio of drug to antibody are called as drug antibody ratio (DAR). In the present disclosure drug to lectin ratio is known as DAR and in an embodiment, DAR may be in the range from about 1 to about 9, wherein DAR may be integer or a non-integer. For example, DAR value can be 2 or 2.5, which may indicate some of the units of lectin may be linked to more than 2 units of cytotoxic agent or therapeutic agent. In an embodiment of the present disclosure DAR may be preferably in the range from about 1 to about 5.

According to an aspect, the DAR value may be determined by the methods known to the skilled person. Determination of the mass of the product formed could be one of the methods to determine DAR value. It will be appreciated by the person skilled in the art that the mass of the product may be determined by chemical analysis or mass spectrometry or other such methods such as liquid chromatograpy mass spectrometry (LCMS). If the mass of the product calculated (by addition of masses of all the atoms in the product) with varying units of cytotoxic agent or therapeutic agent and the analysed mass indicates the mass of particular product with particular units of cytotoxic agent or therapeutic agent, then that is concluded as DAR. For example the mass spectrometry shows peak at mass value that corresponds to mass of lectin protein with 2 units of cytotoxic agent, then the DAR is 2. As understood by the skilled person, there may be more than one peak on mass spectrum indicating different DAR, in such case DAR is determined by the relative percentage of different drug containing species to give an average value.

In an aspect of the present disclosure the bond “—” between L and D may be a covalent bond or a coordinate covalent bond. As understood by the skilled person a ‘covalent bond’ is a bond that is formed between two atoms by sharing of the electron pair. A ‘coordinate covalent bond’ is a covalent bond formed between to atoms, wherein the shared pair of electrons is donated by only one atom. The type of bond formed would depend on both Lectin protein L and D. In a preferred embodiment the bond “+” between L and D is a covalent bond.

According to another aspect, the number of “—” would depend on the DAR value n in Formula I. In some embodiment the value of n for D will be same as value of n for “—”

According to another aspect of the present disclosure the bond “—” between L and D may be a group for formula “A¹-A²-A³”, wherein the formula “A¹-A²-A³” is a linker between L and D and wherein L forms a bond with A¹ and D forms a bond with A³. The bond between L and A¹ and D and A³ may be covalent bond or coordinate covalent bond. In preferred embodiments the bond between L and A¹ and D and A³ is a covalent bond.

The group for formula “-A¹-A²-A³-” is said to be a linker between L and D. It is understood by the skilled person that the linker may be included in the products of the present disclosure for several purposes. For example, the linker is added to decrease steric hindrance between the lectin and the agent D or to make the products more stable or soluble or for the safety of the products for therapeutic use so that the products are not disintegrated in the body before reaching their target or combination thereof.

The linker for the present disclosure may be selected or prepared as per the understanding of the skilled person or using the references such as Linkers having crucial role in antibody drug conjugate (Jun Lu et al., Int. J. Mol. Sci 17, 561, 2016,); Current ADC linker chemistry (Nareshkumar Jain et al., Pharm Res 32: 3526-3540, 2015,); Perspectives about self-Immolative drug delivery systems (Rodrigo et al., Journal of Pharmaceutical Sciences 109, 3263-3281, 2020); Self hydrolysing maleimides improve the stability and pharmacological properties of anti-body drug conjugates (Robert et al., Nature Biotechnology, 32, Number 10, 2004).

In an aspect of the present disclosure, A¹ and A³ is independently selected from: —CR¹R²—, —C(O)—, —C(O)NR¹—, —NR¹C(O)—, —NR¹—, —O—, —S—, —S(O), —S(O)₂—, —R¹C═CR²—, —C≡C—, R¹C═CR²—R³C═CR⁴—, —R¹C═C═CR²—, —C(═NR¹)—,

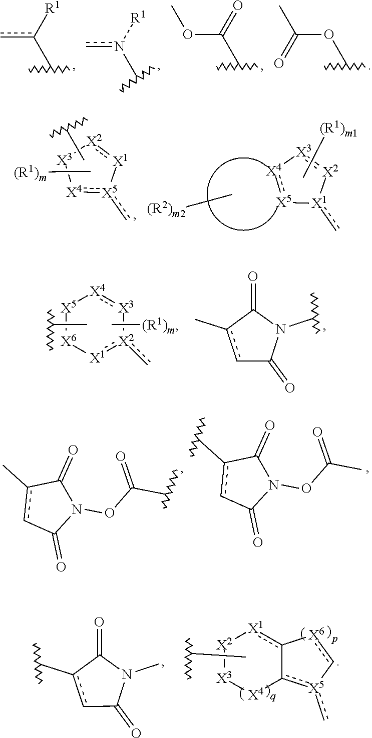

According to an embodiment of the present disclosure, A¹ and A³ may be same or different.

According to another embodiment m, m1 and m2 are independently 0, 1, 2, 3, or 4, wherein m, m1 or m2=0 indicates the substituent R¹ or R² is absent and m, m1, or m2=1 indicates one R¹ or R² present as substituent on the ring and so on form, m1 or m2=2, 3, or 4. It would be appreciated by the skilled person that the value of m, m1, and m2 are independent of each other.

According to yet another embodiment, p is 1 or 2 and q is 0 or 1. When p is 1, the aromatic, cyclic, or non-aromatic ring is 5 membered and when p is 2 the ring is 6 membered. Similarly, when q is 0 the aromatic, cyclic or non-aromatic ring is 5 membered and when its 1, the ring is six membered.

According to an embodiment of the present disclosure “ [symbol] ” indicates the point of attachment of A² to A¹ or A³. It will be obvious to the skilled person that the other terminal of A¹ and A³ is connected to L and D respectively.

According to another aspect, “ [symbol] ” indicates single or double bond. It will be appreciated by the skilled person that unless otherwise mentioned the valency of the atom will be satisfied by addition or removal of the hydrogen on or from atom.

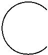

indicates a 5 to 10 membered ring including atoms of the ring it is fused to, wherein the 5 to 10 membered ring is an aromatic, non-aromatic, or cycloalkyl ring or a heteroaromatic or heterocycloalkyl ring wherein the heteroaromatic or heterocycloalkyl rings comprise at least one heteroatom selected from N, O or S.

The atoms are depicted as X¹, X², X³, X⁴, X⁵ and X⁶ are independently selected from C, CH, CH₂, N, NH, O or S. It would be appreciated by the skilled person that all the Xs could be same or different and the number of hydrogen on the X will be adjusted as per its valency and the number of bonds it forms.

For example, in a compound of formula

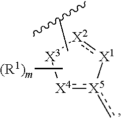

among 5 Xs, 2 Xs may be N or NH and 3 may be C, CH or CH₂. The skilled person is well aware of such interpretations and would understand and consider such variables as an embodiment of the present disclosure.

In A¹ and A³ the cyclic, heterocyclic, aromatic or heteroaromatic compounds can be selected as per the knowledge and understanding of the skilled person. Some of the examples of such compounds are phenyl, benzyl, toluene, naphthyl, cyclohexyl,

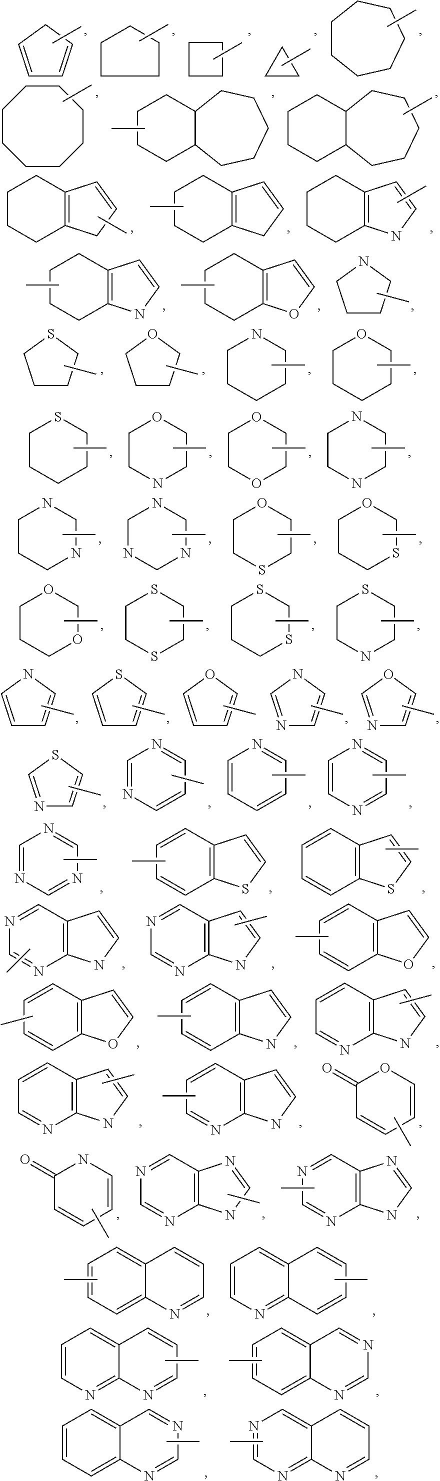

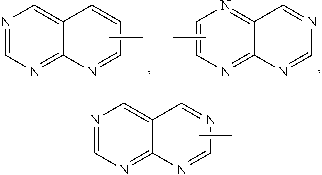

It would be appreciated by the skilled person that the bond shown in the above structure is the bond between D and the structure. The bond with D may be with any carbon or heteroatom if the above structures. The valency of the Carbon and heteroatom would be satisfied by the hydrogen on the same.

In an aspect of the present disclosure, R¹, R², R³, and R⁴ in A¹ and A³ are independently selected from hydrogen, C₁ to C₅ alkyl, C₂ to C₅ alkenyl, C₂ to C₅ alkynyl, C₁ to C₅ alkylene, C₃ to C₁₀ cycloalkyl, C₆ to C₁₀ aryl, C₂ to C₉ heteroaryl, —OR⁵, —NR⁵—SR⁵, halogen selected from fluorine, chlorine, bromine or iodine, —C(O)OR⁵, wherein C₁ to C₅ alkyl, C₂ to C₅ alkenyl, C₂ to C₅ alkynyl, C₁ to C₅ alkylene, C₃ to C₁₀ cycloalkyl, C₂ to C₉ heterocycloalkyl, C₆ to C₁₀ aryl, C₂ to C₉ heteroaryl is further substituted by R⁶, and R⁵ and R⁶ is hydrogen, C₁ to C₃ alkyl, OH, or halogen.

According to one aspect, A¹ and A³ may be selected from —CR¹R²—, —C(O)—, —NR¹—, —S—,

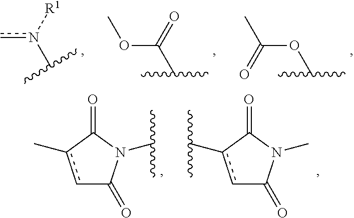

wherein R¹, R², “ [symbol] ” and “ [symbol] ” are same as defined above.

According to another more preferred aspect, A¹ may be —C(O)—.

According to an aspect of the present disclosure, A² in group “-A¹-A²-A³” is of the formula:

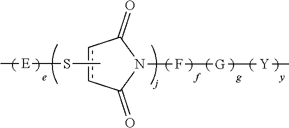

In an aspect, e, j, f, g and y is independently either 0 or 1. It would be appreciated by the skilled person that whenever e, j, f, g and y are 0, the corresponding groups in A² are absent. According to a specific aspect of the present disclosure ‘e’, ‘f’0 and ‘y’ are not 0 simultaneously, and therefore the groups E, F and Y cannot be absent in a single product of formed according to this aspect. A¹ in the present disclosure forms a bond with E or S or F and A³ is connected to Y or G.

According to an aspect, S in A² is a sulphur atom and E, F and Y is a spacer. The spacer when present, extends the framework of the Linker unit to provide more distance between the lectin protein and the agent D. The spacer can also act to alter the physiochemical properties of the final products of the present disclosure depending on components of the spacer. The spacer may be added in order to increase the solubility of the product and may comprise one or multiple solubility-enhancing groups such as ionic groups or water-soluble polymers. Water-soluble polymers typically include any segment or polymer that is soluble in water at room temperature and includes poly(ethylene)glycol groups as well as other polymers such as polyethyleneimines.

In an aspect of the present disclosure, each spacer E, F and Y is independently selected from —R⁷—, —S—, —NH—, —R⁷—N—R⁸—, R⁸—NC(O)—R⁷—, —R⁸—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, —O—R⁷—, —R⁷—O—, —R⁸—O—R⁷—, —C(O)O—, —OC(O)—R⁷—, —R⁷—OC(O)—, R⁸—OC(O)—R⁷—, —R⁷—OC(O)O, —OC(O)O—R⁷—, —R⁷—OC(O)O—R⁸—, —NH—R⁷—NH C(O)R⁸—, —(CH₂)S—NH—R⁷—, —R⁷—C(O)N(CH₂)₁₋₃O(CH₂)₁₋₃C(O)—, —R⁷—NC(O)(CH₂)₁₋₃O(CH₂)₁₋₃C(O)—, —Si(R⁷R⁸)—, polyalkylene glycol optionally attached through oxygen to —R⁷—, —S—, —NH, —R⁷—N—R⁸—NC(O)—R⁷—, —R⁸—C(O)N—R⁷—, —C(O)—, —C(O)—R⁷—, —R⁷—C(O)—, —R—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, O—R⁷—, —R⁷—O—, —R⁸—O—R⁷—C(O)O—, —OC(O)—R⁷—, —R⁷—OC(O)—, R⁸—OC(O)—R⁷—, —R⁷—OC(O)O—, —OC(O)O—R⁷—, —R⁷—OC(O)O—R⁸N—, —NH—R⁷—NH—C(O)R⁸—, —(CH₂)S—NH—R⁷—, —R⁷—C(O)N(CH₂)₁₋₃O(CH₂)₁₋₃C(O)—, —R⁷—NC(O)(CH₂)₁₋₃O(CH₂)₁₋₃C(O)—, —Si(R⁷R⁸)—.

According to one preferred embodiment, E may be selected from —R⁷—N—R—R⁸—NC(O)—R⁷—, —R—C(O)N—R⁷—, —C(O)—R⁷—, —R⁷—C(O)—R—C(O)—R⁷—, polyalkylene glycol optionally attached through oxygen to —R⁷—N—R—R⁸—NC(O)—R⁷—, —R—C(O)N—R⁷—, —C(O)—R⁷—, —R⁷—C(O)—R—C(O)—R⁷—.

In an embodiment, R⁷ and R⁸ in E, F or Y is independently selected from hydrogen, —C₁-C₁₀ alkylene, —C₁-C₁₀ alkenylene, C₅ to C₁₀ saturated or unsaturated cycloalkylene, —C₁-C₁₀ alkenylene-C₅ to C₁₀ saturated or unsaturated cycloalkylene, C₅ to C₁₀ arylene, —C₁-C₁₀ alkenylene-C₆ to C₁₀ arylene, C₂ to C₉ heteroarylene, —C₁-C₁₀ alkenylene-C₂ to C₉ heteroarylene; and wherein —C₁-C₁₀ alkylene, —C₁-C₁₀ alkenylene, C₅ to C₁₀ saturated or unsaturated cycloalkylene, —C₁-C₁₀ alkenylene-C₅ to C₁₀ saturated or unsaturated cycloalkylene, C₅ to C₁₀ arylene, —C₁-C₁₀ alkenylene-C₆ to C₁₀ arylene, C₂ to C₉ heteroarylene and —C₁-C₁₀ alkenylene-C₂ to C₉ heteroarylene is optionally further substituted with —OH, —C(O), —SH, —NH₂ halogen, C₁ to C₅ alkyl, —NO₂, —CN. The definitions and meaning of terms alkylene, alkenylene, cycloalkylene, heteroarylene are known to the skilled person or can be understood as defined hereinbefore.

In another embodiment, polyalkylene glycol in E, F or Y may be a homopolymer or copolymer of alkylene with each unit of alkylene having C₁ to C₈ carbon atoms and total number of alkylene units ranging from 1 to 20. In a preferred embodiment each unit of alkylene may range from C₂ to C₅ and the alkylene units may be in the range from 1 to 15.

According to an aspect when polyalkylene glycol is E the —CH₂— end is attached to A¹ and when polyalkylene glycol is F the —CH₂— end is attached to —R⁷—, —S—, —NH—, —R⁷—N—R⁸—, —R⁸—NC(O)—R⁷—, —R⁸—C(O)N—R⁷—, —C(O)—, C(O)—R⁷—, —R⁷—C(O)—, —R—C(O)—R⁷—, —S—R⁷—, —R⁷—S—, —R⁷—S—R⁸—, S(O), —S(O)—R⁷—, —R⁷—S(O)—, —R⁷—S(O)—R⁸—, O—R⁷—, —R⁷—O—, —R⁸—O—R⁷—, —C(O)O—, —OC(O)—R⁷—, —R⁷—OC(O)—, —R⁸—OC(O)—R⁷—, —R⁷—OC(O)O—, —OC(O)O—R⁷—, —R⁷—OC(O)O—R⁸—, —NH—R⁷—NH—C(O)R⁸— or —(CH₂)S—NH—R⁷—, —R⁷—C(O)N(CH₂)₁₋₃O(CH₂)₁₋₃C(O)— or —R⁷—NC(O)(CH₂)₁₋₃O(CH₂)₁₋₃C(O)—.

In some aspects of the present disclosure, A¹ and E or A³ and Y may combine to form a single unit. In such a situation it might be difficult to specify and differentiate between A¹ and E or A³ and Y. Such situations are well within the understanding and ambit of the knowledge of the skilled person.

In some aspects of the present disclosure the spacer unit F and Y may comprise a cleavable unit. In another aspect F and Y may be linked by a cleavable unit such as G. The cleavable unit is capable of forming a cleavable bond in the linker. Such bond may be formed with spacer Y or with A³. In an embodiment in the absence of Y or A³, a cleavable bond is formed directly with agent D.

Reactive groups for forming cleavable bonds can include, for example, sulfhydryl groups to form disulfide bonds, aldehyde, ketone, or hydrazine groups to form hydrazone bonds, carboxylic or amino groups to form peptide bonds, and carboxylic or hydroxy groups to form ester bonds. The nature of the cleavable unit can vary widely. For example, cleavable linkers include disulfide containing linkers that are cleavable through disulfide exchange, acid-labile linkers that are cleavable at acidic pH, and linkers that are cleavable by enzymes such as hydrolases, peptidases, esterases, and glucoronidases. The cleavable unit can comprise one or multiple cleavage sites.

In some embodiments of the present disclosure, the cleavable unit is G. G may comprise one amino acid or one or more sequences of amino acids. In a particular embodiment G is a peptide chain comprising 2, 3, 4, 5, 6, 7, 8, 9, 10. 11 or 12 amino acids. Thus, G may be a monopeptide, a dipeptide, tripeptide, tetrapeptide, pentapeptide, hexapeptide, heptapeptide, octapeptide, nonapeptide, decapeptide, undecapeptide or dodecapeptide unit. Each amino acid can be natural or unnatural and/or a D- or L-isomer provided of course that there is a cleavable bond. In some embodiments, the G may comprise only natural amino acids. In some aspects, the G may comprise 1 to 12 amino acids in contiguous sequence.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In another embodiment, each amino acid is independently selected from the group consisting of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan, and valine.

In another embodiment, each amino acid is independently selected from the group consisting of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan, and valine.

In certain embodiments, G can comprise only natural amino acids. In other embodiments, G can comprise only non-natural amino acids. In some embodiments, G can comprise a natural amino acid linked to a non-natural amino acid. In some embodiments, G can comprise a natural amino acid linked to a D-isomer of a natural amino acid.

In some embodiments Y and A³ independently or in combination may comprise multiple self-immolative or non-self immolative groups. In this context, the term “self-immolative group” refers to a bifunctional chemical moiety that is capable of covalently linking together two spaced chemical moieties into a normally stable tripartite molecule. It will spontaneously separate from the second chemical moiety if its bond to the first moiety is cleaved.

In some embodiment of the present disclosure, E and F may be a non-cleavable unit, which is capable of linking a cytotoxic agent or a therapeutic agent to a recombinant lectin protein in a generally stable and covalent manner and is substantially resistant to acid-induced cleavage, light-induced cleavage, peptidase- or esterase-induced cleavage, and disulfide bond cleavage. Cytotoxic agent or therapeutic agent is released from the products of present disclosure containing non-cleavable unit via alternative mechanisms, such as proteolytic ligand degradation. In some embodiment of the present disclosure, non-cleavable unit may comprise a maleimido moiety or a moiety selected from but not limited to N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), c-maleimidocaproic acid N-hydroxysuccinimide ester (EMCS), m-maleimidobenzoyl-N-hydroxysuccinimide ester (MBS), N-(.alpha.-maleimidoacetoxy)-succinimide ester [AMAS], succinhnidyl-6-(P-maleimidopropionamido)hexanoate (SMPH), N-succinimidyl 4-(p-maleimidophenyl)-butyrate (SMPB), and N-(p-maleimidophenyl)isocyanate (PMPI), N-succinimidyl-4-(iodoacetyl)-aminobenzoate (STAB), N-succinimidyl iodoacetate (SIA), N-succinimidyl bromoacetate (SBA) and N-succinimidyl 3-(bromoacetamido)propionate (SBAP).

In a second aspect of present disclosure, there is provided a compound of formula II:

L-D Formula II
or a pharmaceutically acceptable salt thereof, wherein
L and D are as defined herein before.

According to a specific aspect of the present disclosure, L is a recombinant protein selected from SEQ ID NO: 2, SEQ ID NO: 3, SEQ ID NO: 4 and SEQ ID NO: 5.

According to a more particular aspect, L is a recombinant protein having amino acid sequence of SEQ ID NO: 5.

According to an aspect the bond “—” between L and D is a covalent bond or coordinate covalent bond. According to another aspect the bond “—” between L and D is a group for formula “-A¹-A²-A³”, wherein A¹, A² and A³ are as defined above.

According to an aspect, DAR value for Formula II is 1. Thus, each unit of Formula II comprise one unit of lectin protein and one unit of cytotoxic agent or therapeutic agent.

In one embodiment, a compound of Formula II is;

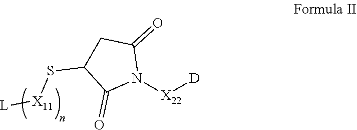

wherein,
L is a recombinant lectin derived from S. rolfsii;
D is a Drug or a therapeutic agent.
S is a Sulphur atom;
X₁₁ is a group comprising:
   a. a group selected from an amine reactive group or sulfhydryl-reactive group;
   b. optionally a hydrophilic polymer;
   c. a stretcher, wherein S of Formula III is attached to the stretcher;
X₂₂ is a group comprising:
   a. amine-reactive group, sulfhydryl-reactive group, hydroxyl reactive group or carbonyl reactive group
   b. optionally a peptide having 2 to 10 amino acids sequence
   c. optionally a hydrophilic polymer;
   d. a stretcher;
wherein the amine reactive group, sulfhydryl-reactive group, hydroxyl reactive group, carbonyl reactive group hydrophilic polymer and peptide is as claimed in any of the preceding claims; and
wherein stretcher comprises one or more than one groups selected from R³, —NR³R⁴, —NHR³C(O)R⁴—, —NH—R³C(O)—NHR⁴, —NH—(CH₂CH₂O)S—, —NH—(CH₂CH₂O)S—CH₂—, —NH—(CH₂CH₂NH)S—(CH₂)S—NH—(CH₂CH₂NH)S—(CH₂)S—NH—C(O)—(CH₂)S—NH—(C₃-C₈ carbocyclo)-, —NH-(aryl-)—, —NH—(C₃-C₈ heterocyclo-)—, —R³C(O)—, —O(CO)R³—, —O(CO)R³NH—, —O(CO)R³SR⁴—, —O(CO)R³—SR⁴CO—NR³R⁴—, C₃-C₈ carbocyclo-, —C₃-C₈ carbocyclo-CO—NHR³—, —C₃-C₈ carbocyclo-CO—NHR³C₃-C₈ heterocyclo-, —OCO(C₃-C₈ carbocyclo)-, —OCO(C₃-C₈ carbocyclo)-R³—, —OCO(C₃-C₈ carbocyclo)-R³—CO—NR³R⁴.
R³ and R⁴ is selected from hydrogen, C₁-C₁₀ alkyl, C₁-C₁₀ alkelene, C₁-C₁₀ alkylene, C₆-C₁₀ aryl, C₃-C₈ heteroaryl, C₃-C₈ carbocyclyl, wherein alkyl, alkelene and alkylene is straight or branched chain, and wherein C₁-C₁₀ alkyl, C₁-C₁₀ alkelene, C₁-C₁₀ alkylene, C₆-C₁₀ aryl, C₃-C₈ heteroaryl, C₃-C₈ carbocyclyl is further substituted by R⁶, wherein R⁶ is Oxo.

In one of the embodiments, the compound of formula II is prepared as below steps:
a. reaction of recombinant lectin protein (L) with precursor of linker X₁₁—S-A at room temperature for 60 minutes to produce L-X₁₁—S-A, A is a good leaving group;
b. reaction of L-X₁₁—S-A as prepared in step a) with compound of below formula IIIA to form recombinant lectin Drug conjugate of Formula II

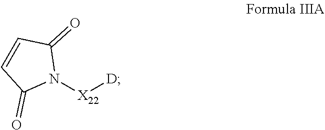

c. purification of compound of Formula II.

In a third aspect of the present disclosure, there is provided a compound of Formula III:

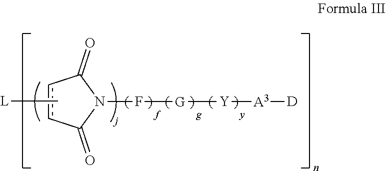

or a pharmaceutically acceptable salt thereof, wherein
L, F, G, Y, A³, D, n, j, f, g and y are as defined herein before.

According to an aspect, the lectin protein L is directly linked to succinimide or maleimide group via its free amino, thiol, acid or hydroxy group. More preferably L is linked to succinimide or maleimide group via Sulphur of thiol group present in cysteine.

According to a fourth aspect of the present disclosure there is provided a compound of Formula IV:

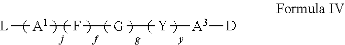

or a pharmaceutically acceptable salt thereof, wherein
L, A¹, F, G, Y, A³, D, j, f, g and y are as defined herein before.

According to an aspect, A¹ in Formula IV may be present when j is 1 or absent when j is 0. L is directly linked to F in the absence of A¹. As defined above j, f, g and y can be 0 or 1. In an embodiment s, f and y cannot be 0 in a single compound of Formula IV. In another embodiment j and f cannot be 0 together and f and y cannot be 0 together. In the absence of Y, A³ may be directly linked to G or F and in the absence of F, A¹ may be directly linked to G or Y.

In some aspects, compounds of Formula I, II, III, IV have a DAR of about 1 to about 2. In some aspects, compounds of Formula I, II, III, IV have a DAR of about 1 to about 1.5. In some aspects, compounds of Formula I, II, III, IV have a DAR of about 1. In some aspects, compounds of Formula I, II, III, IV have a DAR of about 2.

Compounds of this disclosure may have one or more asymmetric centres. Unless otherwise indicated, all chiral (enantiomeric and diastereomeric) and racemic forms of compounds of the present disclosure are included in the present disclosure. Many geometric isomers of olefins, C═N double bonds, and the like can also be present in the compounds, and all such stable isomers are contemplated in the present disclosure. Cis and trans geometric isomers of the compounds of the present disclosure are described and may be isolated as a mixture of isomers or as separated isomeric forms. The present compounds can be isolated in optically active or racemic forms.

The compounds of this disclosure include all conformational isomers (e.g., cis and trans isomers). The compounds of the present disclosure have asymmetric centres and therefore exist in different enantiomeric and diastereomeric forms. This disclosure relates to the preparation and use of all optical isomers and stereoisomers of the compounds of the present disclosure and to all pharmaceutical compositions and methods of treatment that may employ or contain such isomers. In this regard, the disclosure includes both the E and Z configurations. The compounds of present disclosure may also exist as tautomer. This disclosure includes all such tautomers. In one embodiment of the present disclosure, the use of compound as represented in formula I-IV for the treatment or prevention or cure of cancer or tumor.

Exemplarily, some of the compounds of present disclosure are compound of Formula 1 to Formula 43.

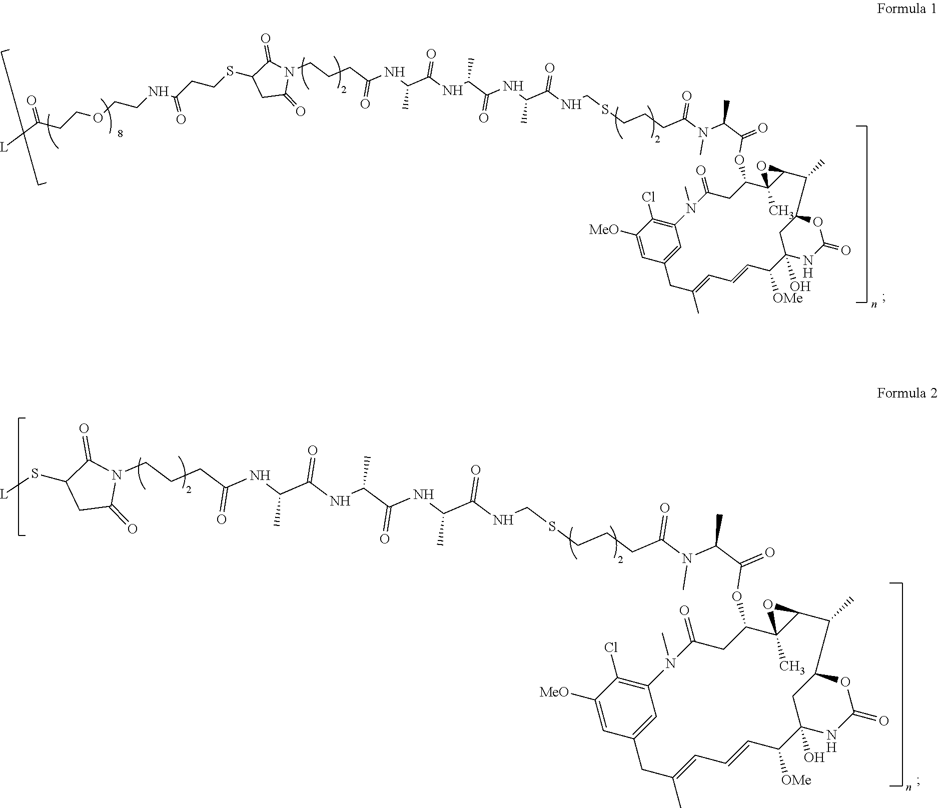

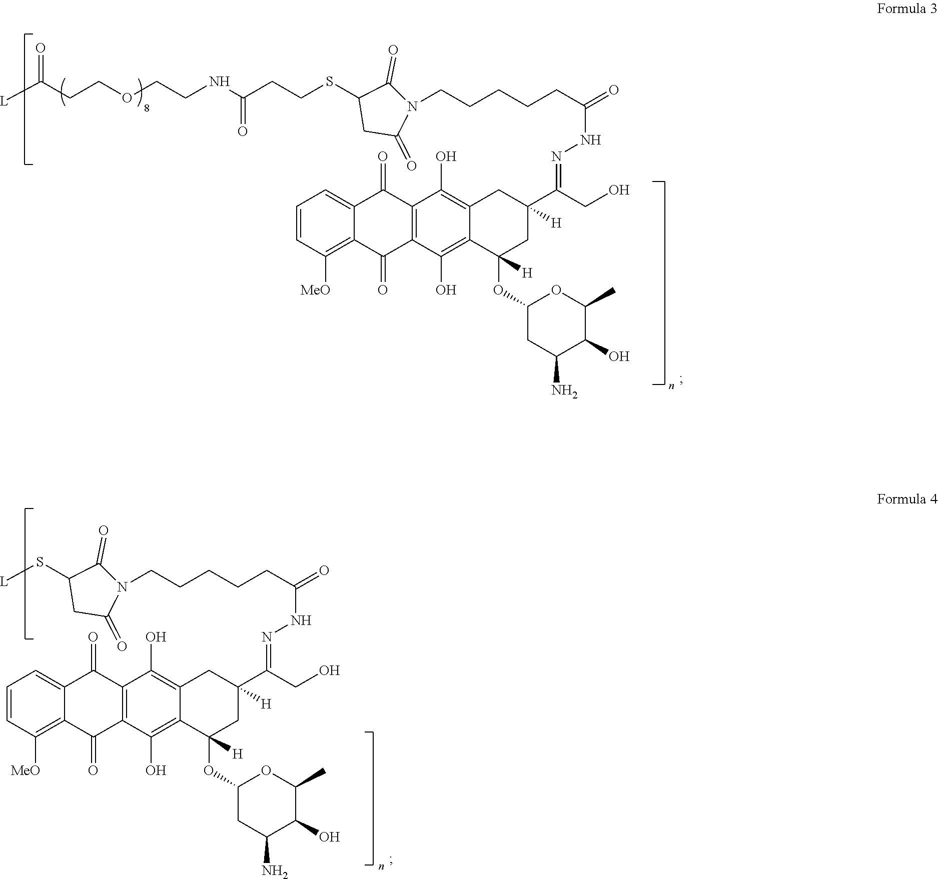

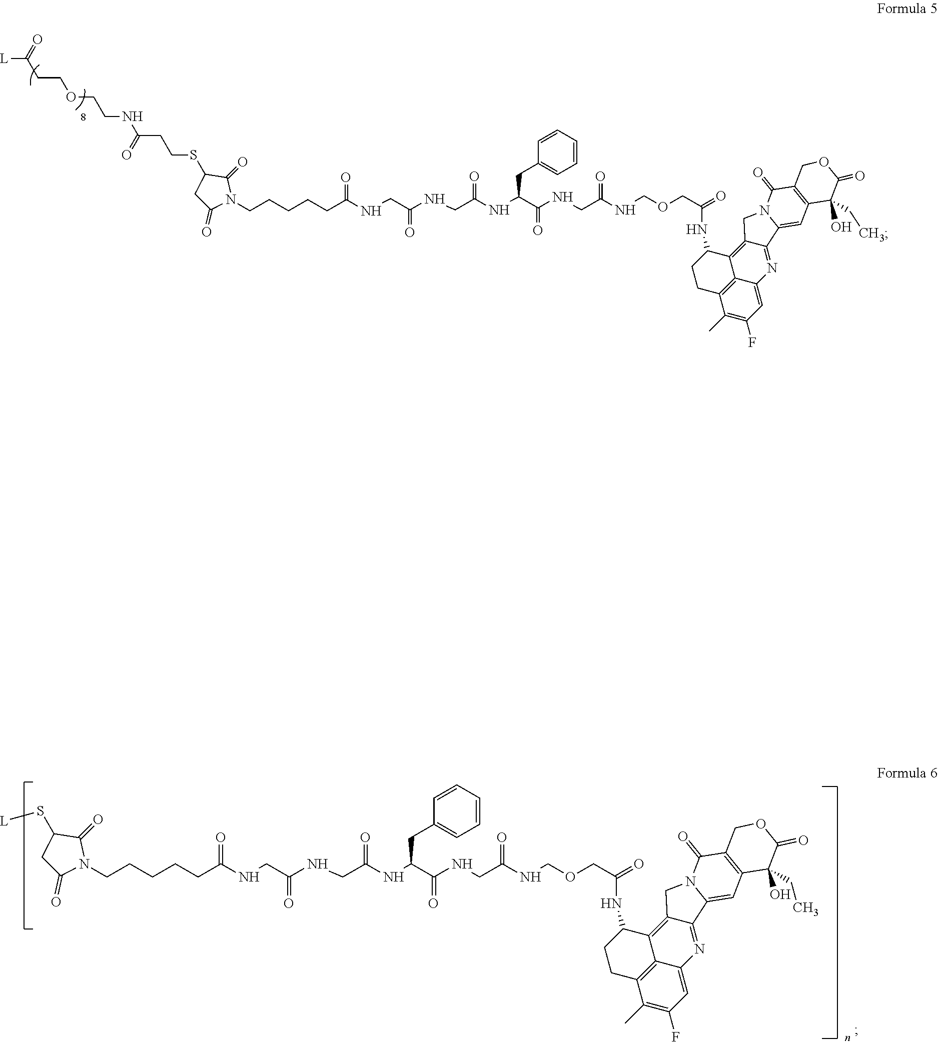

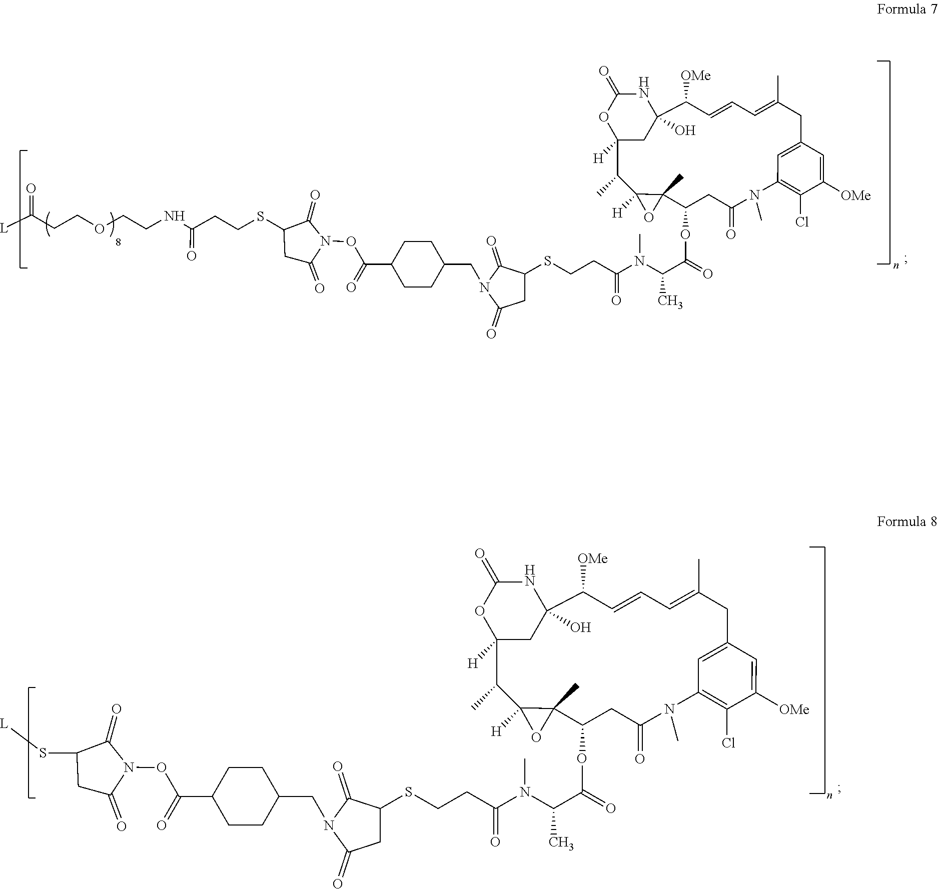

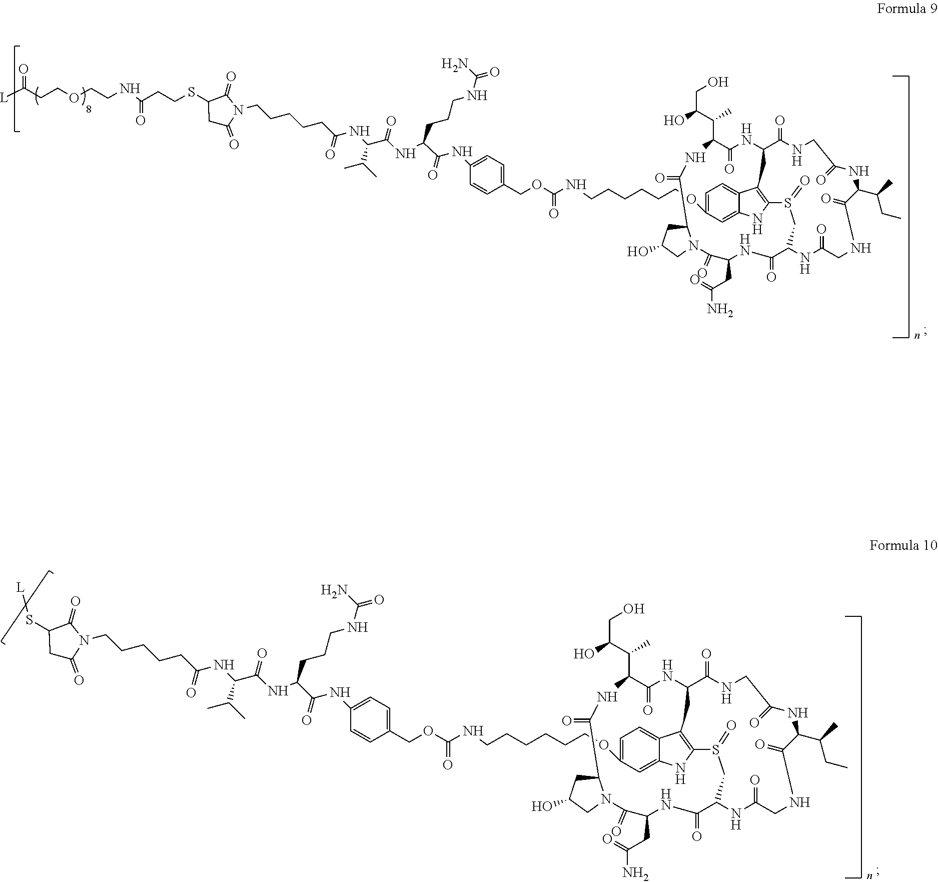

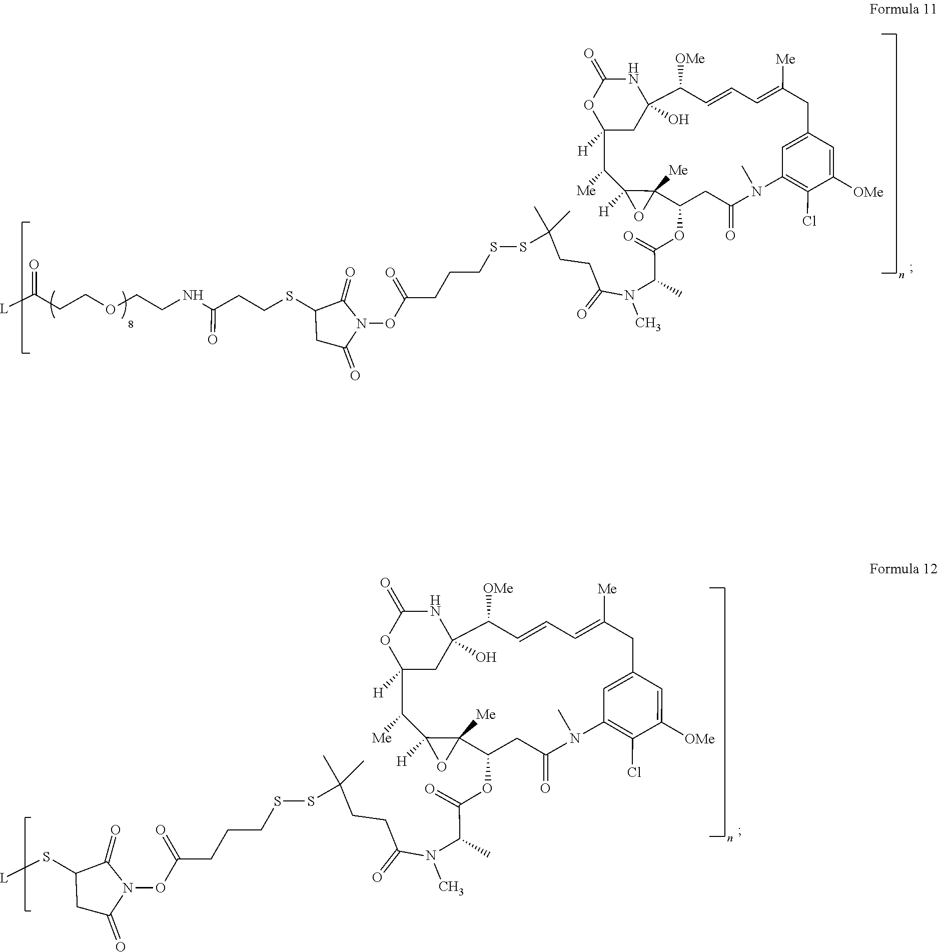

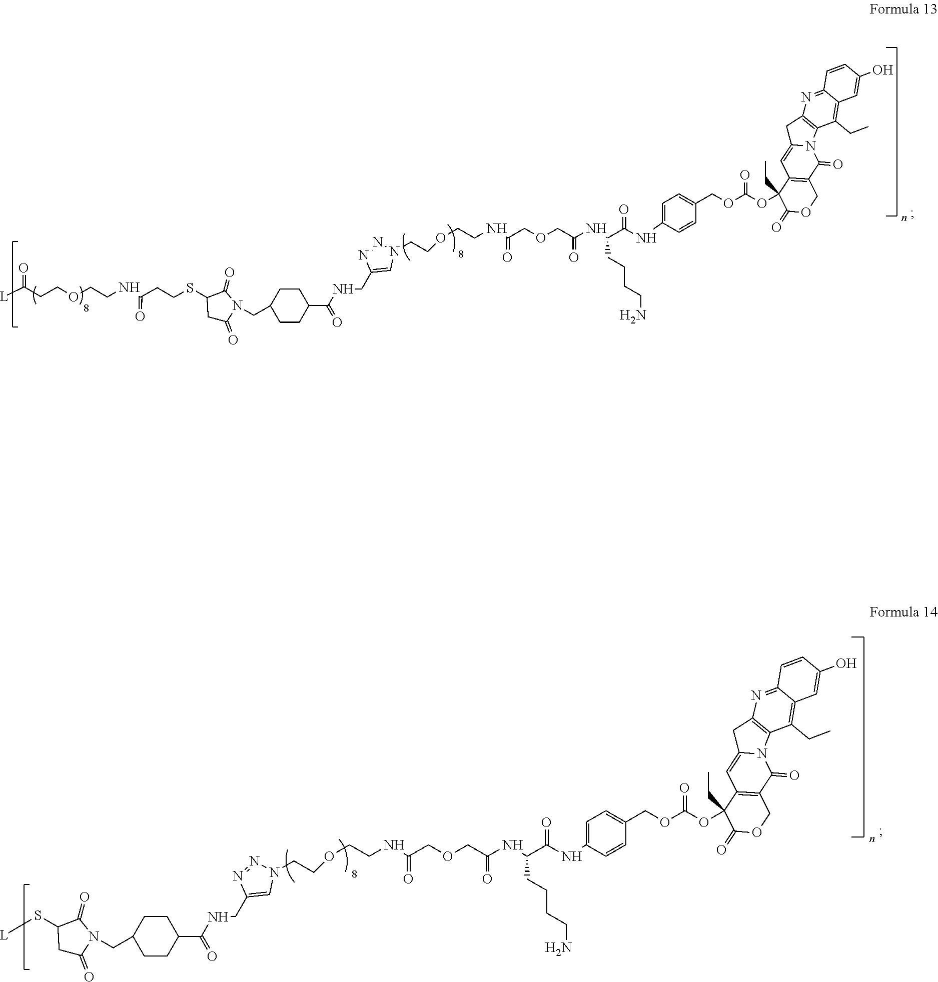

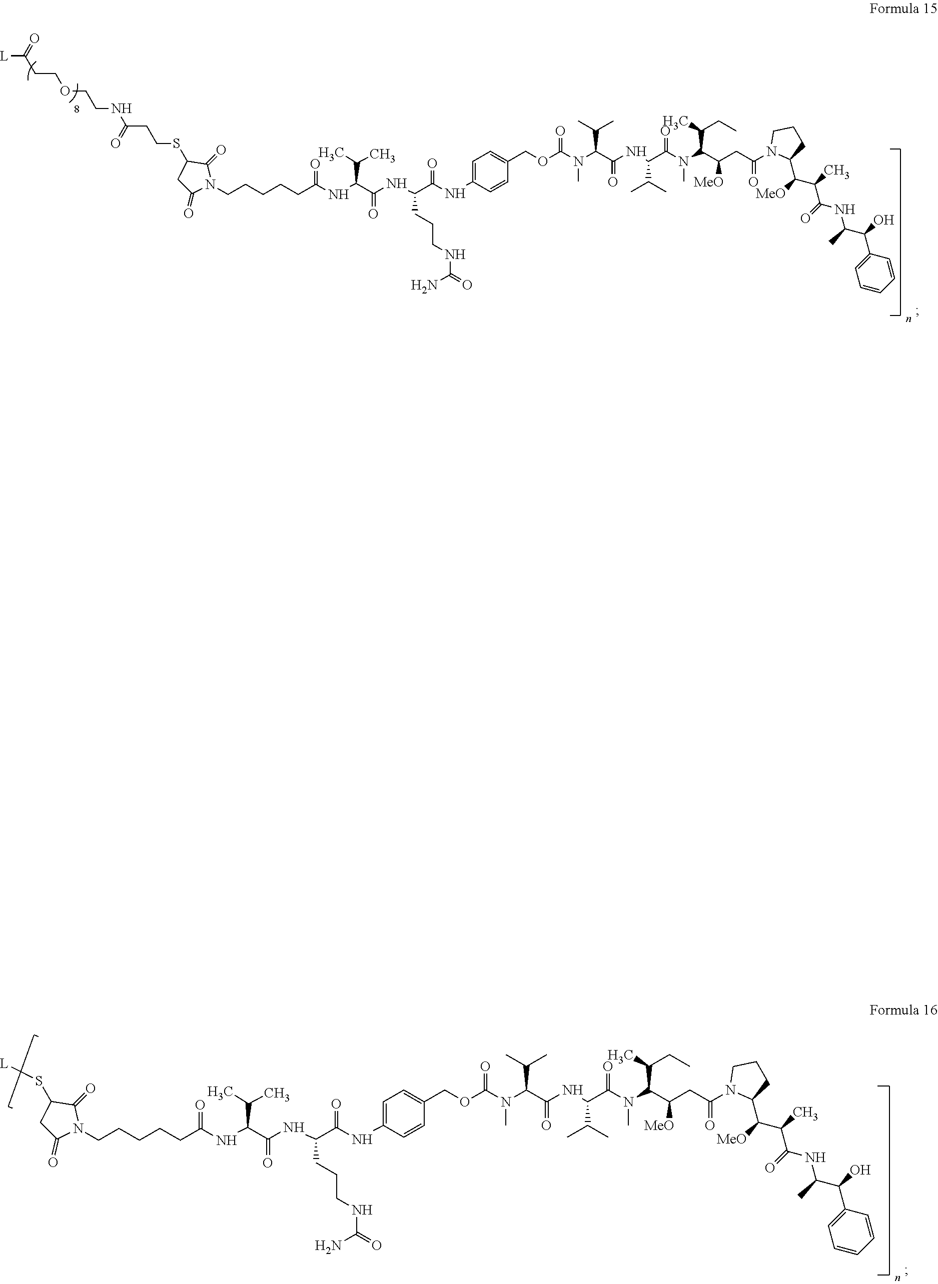

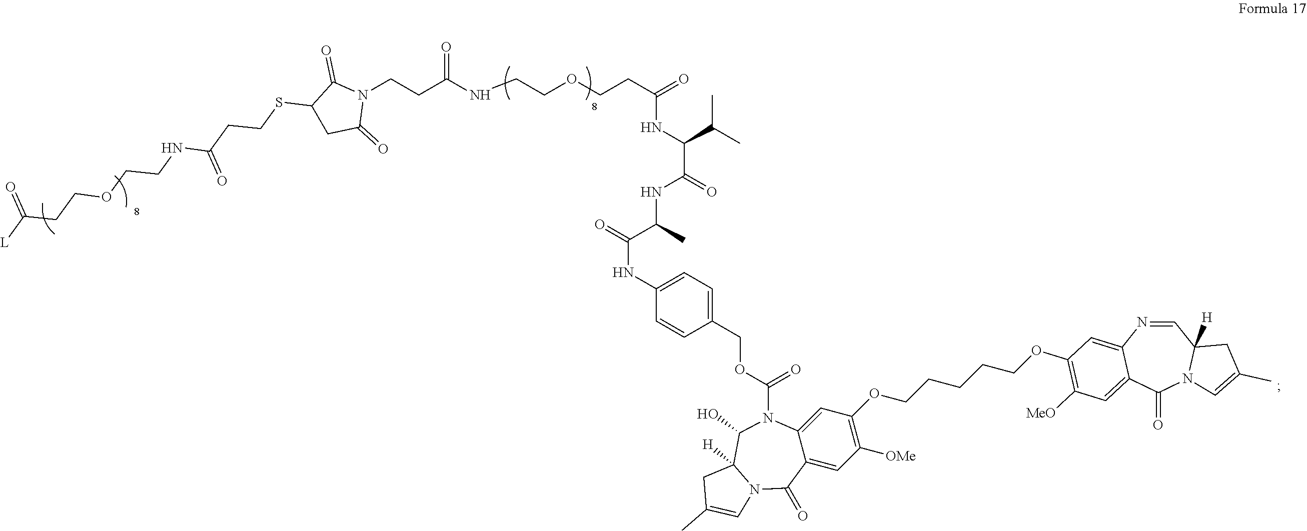

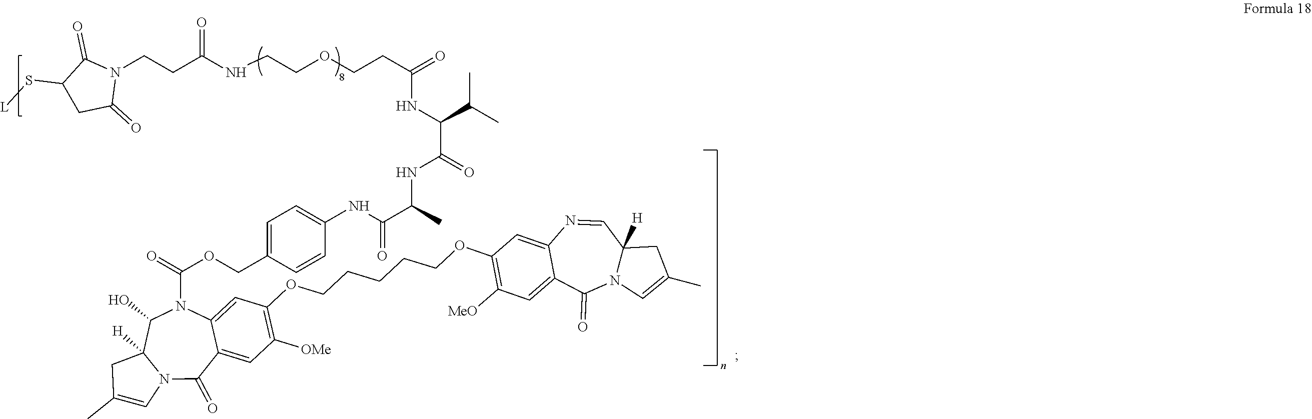

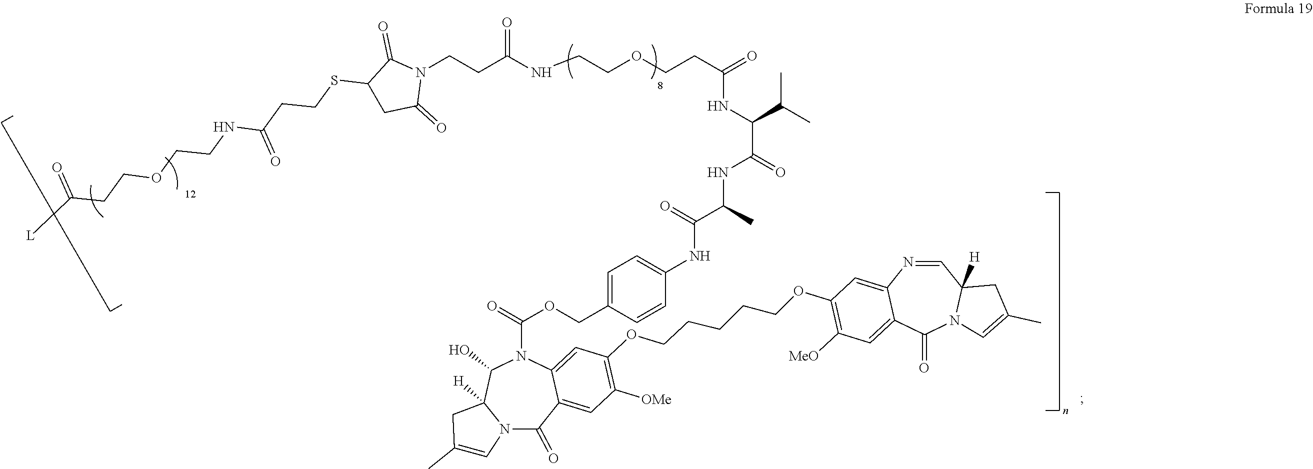

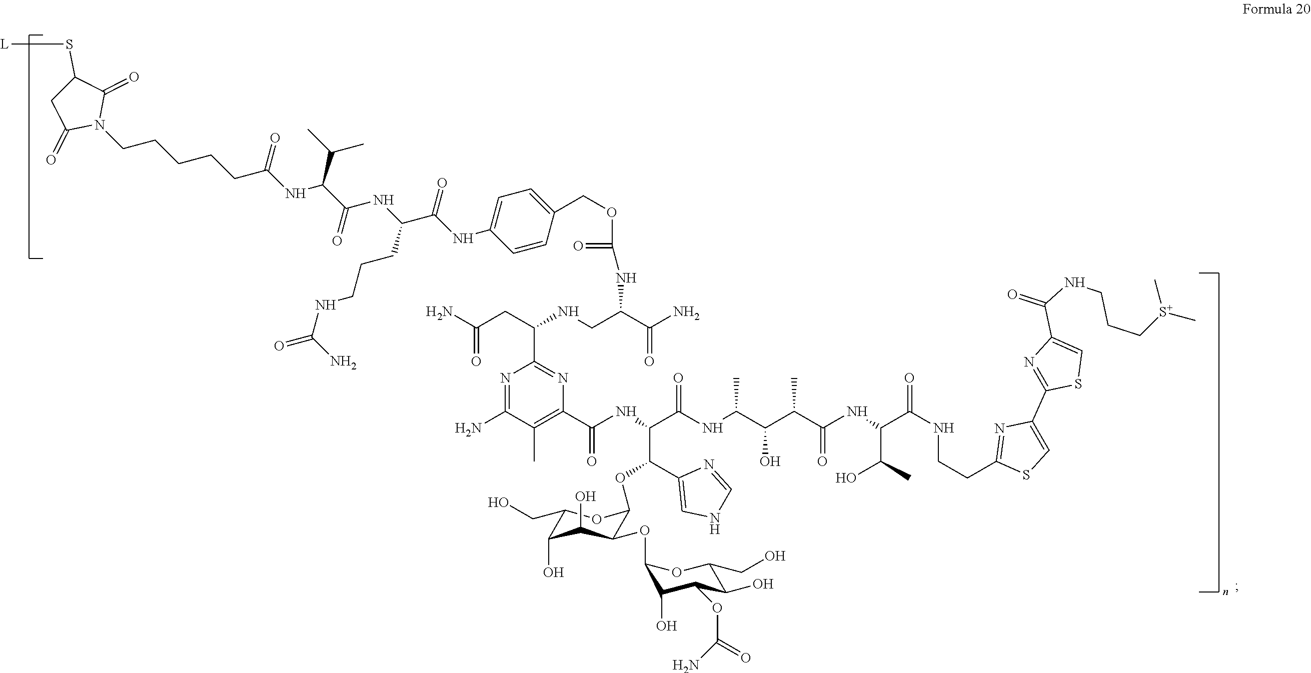

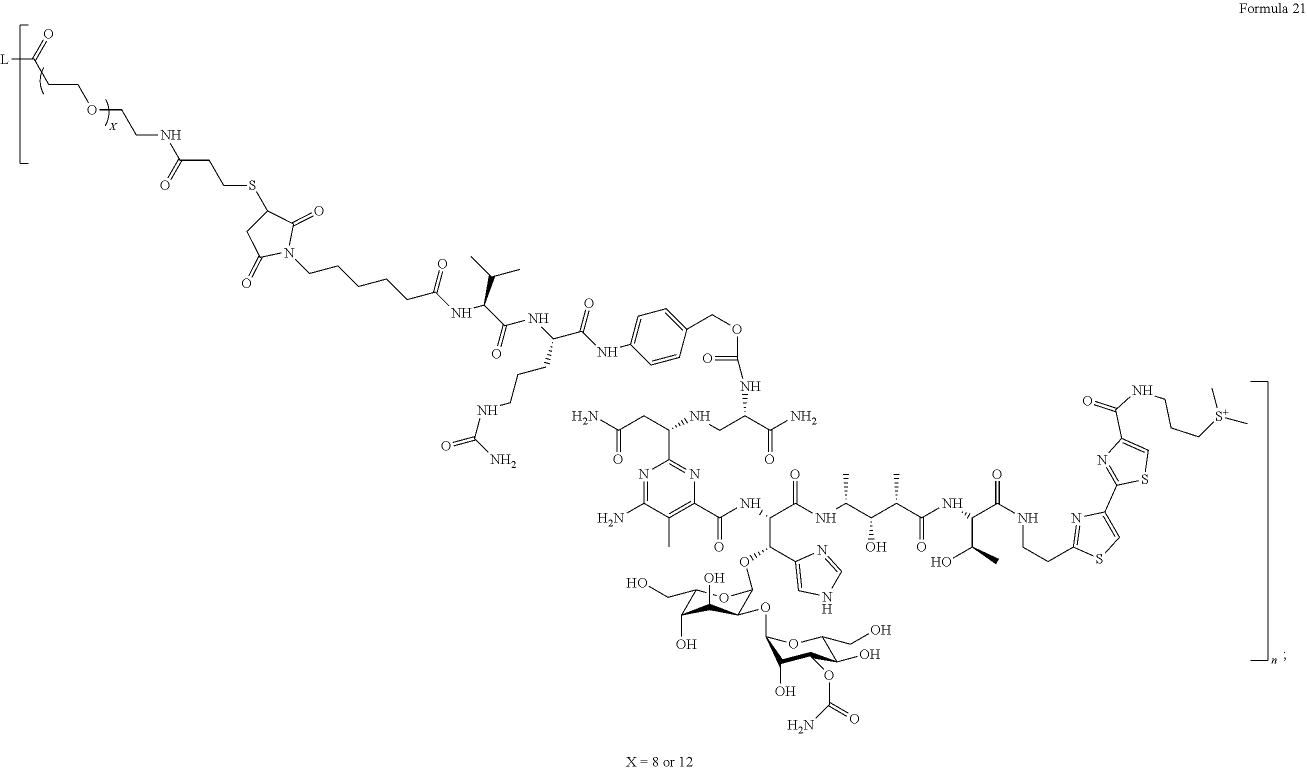

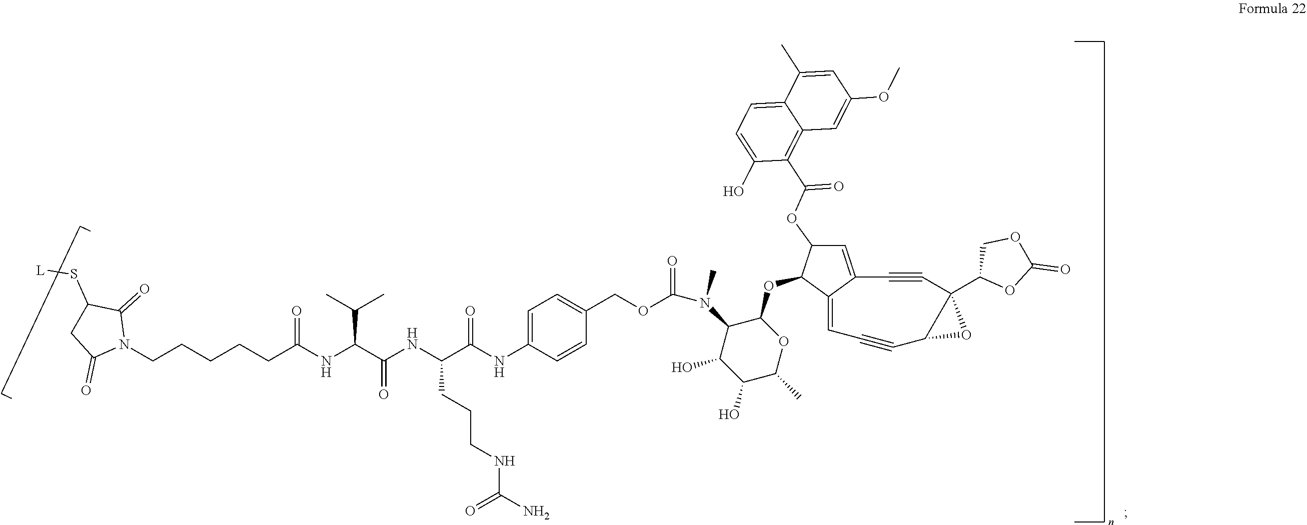

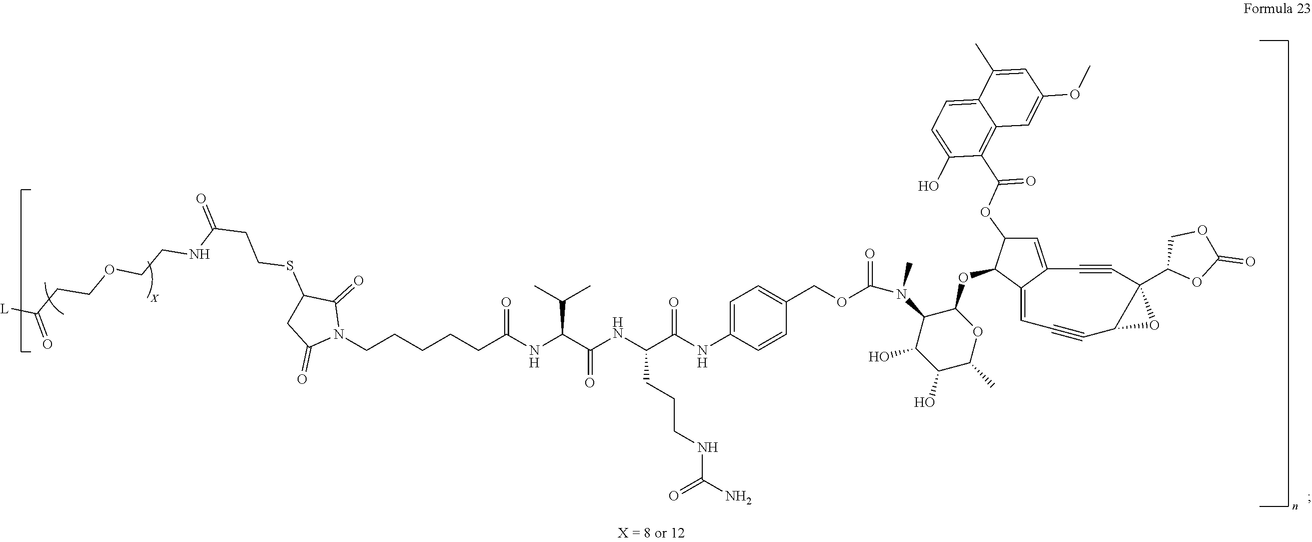

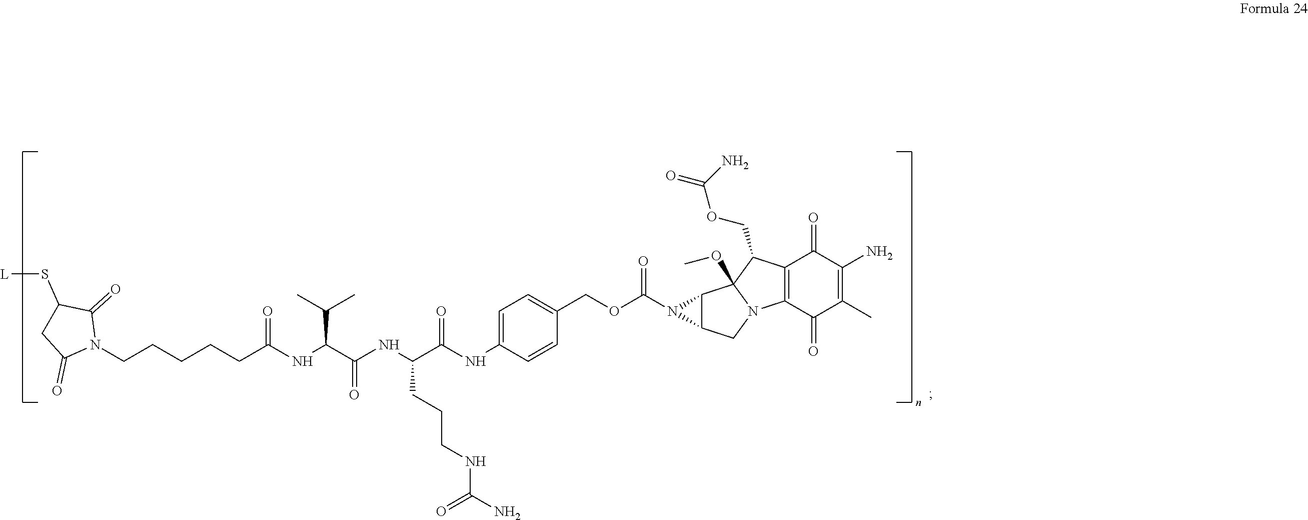

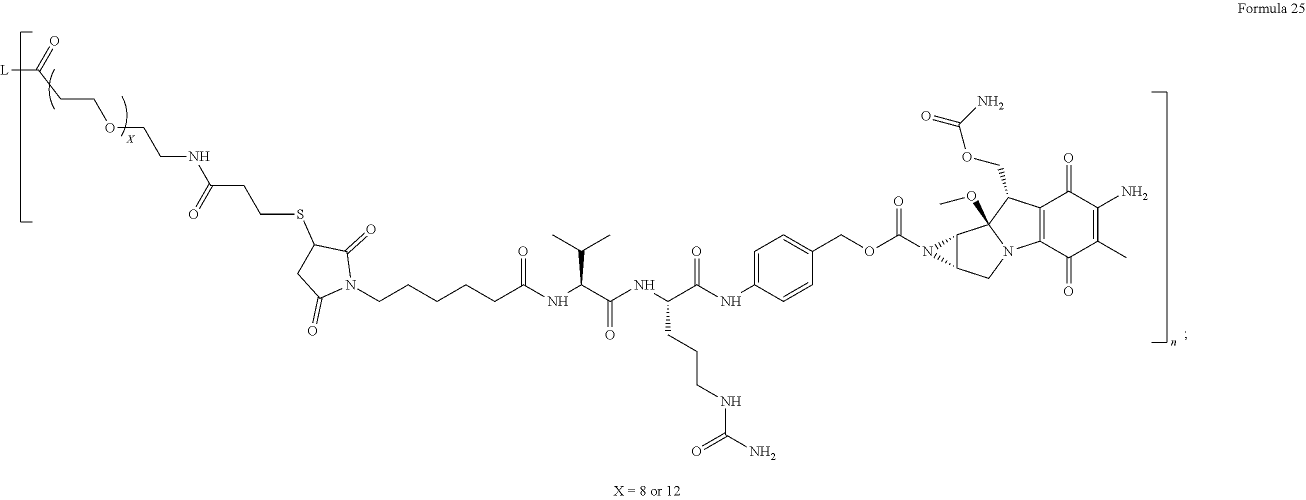

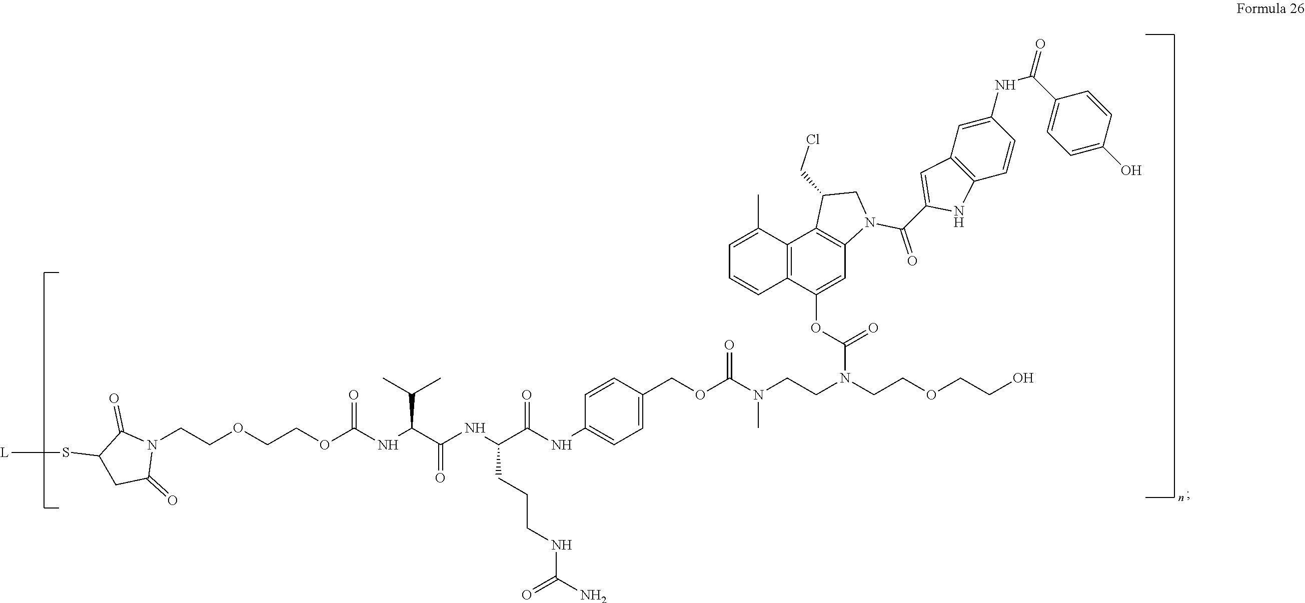

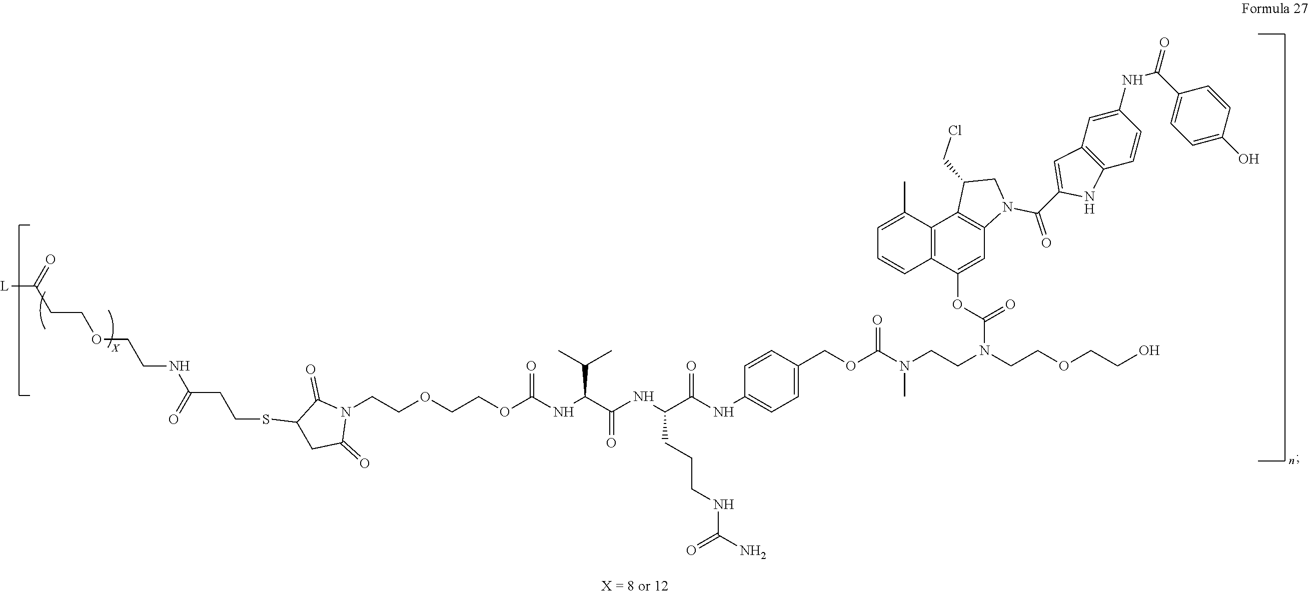

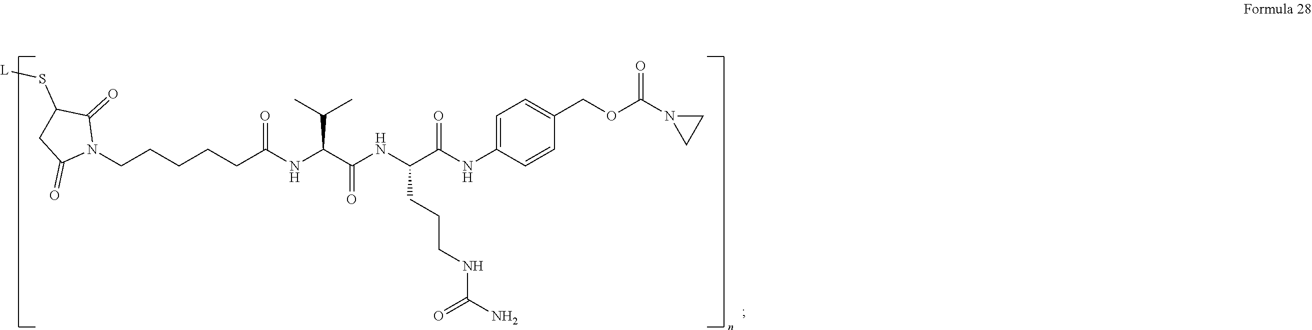

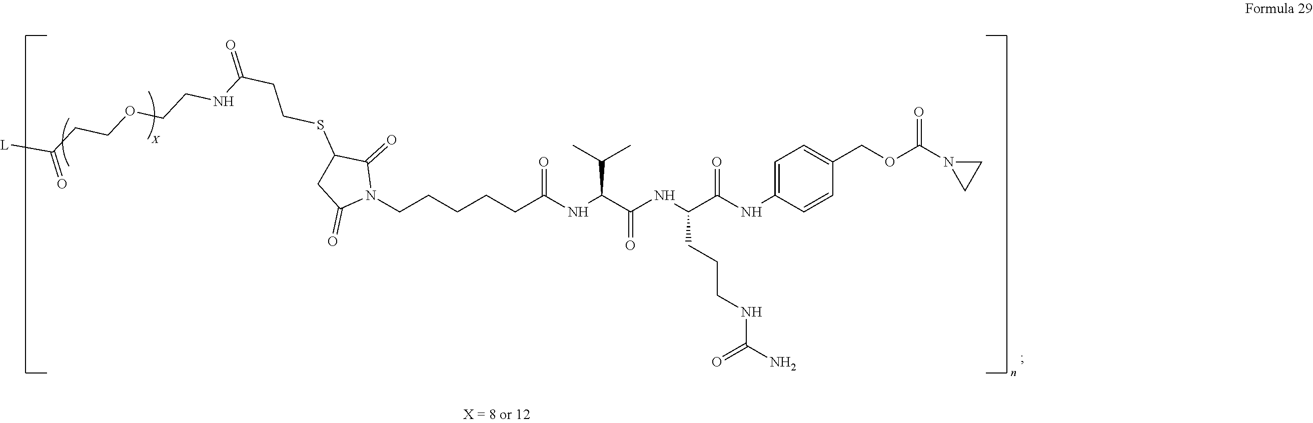

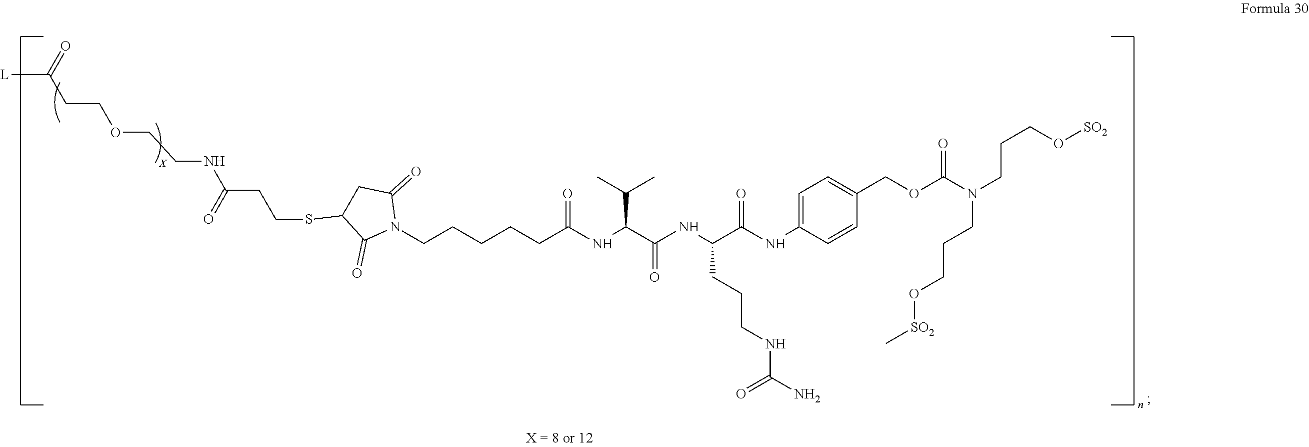

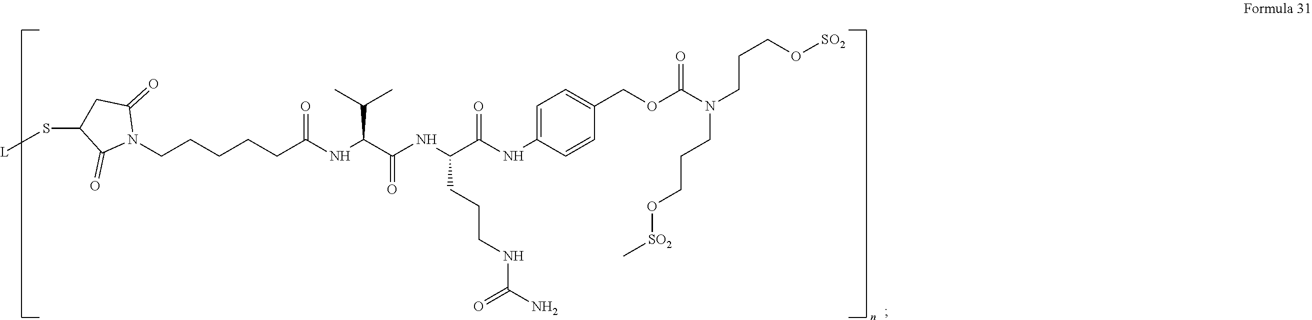

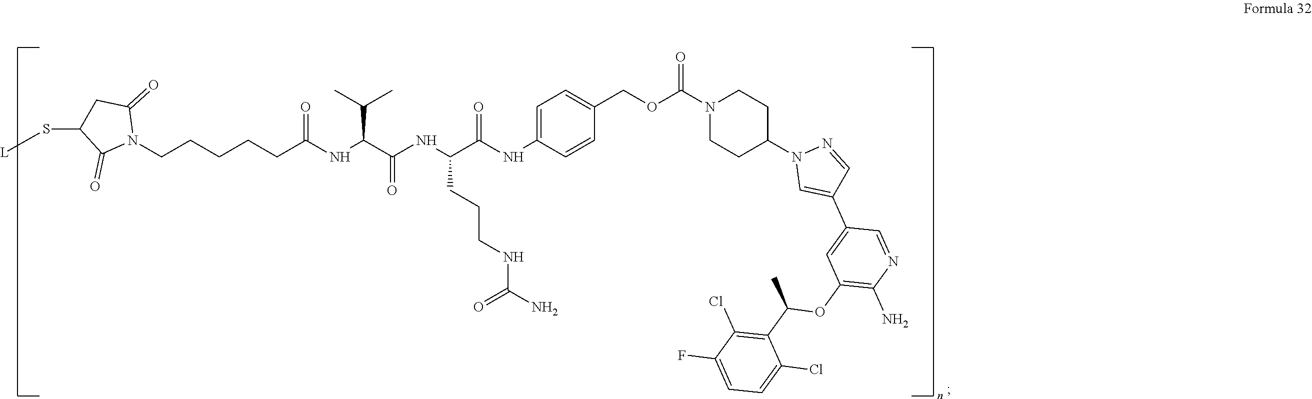

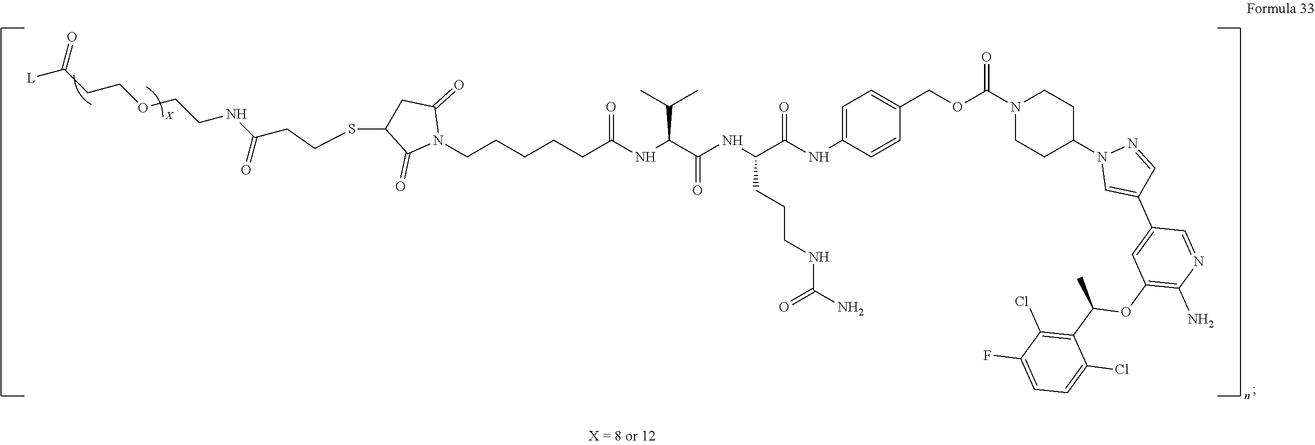

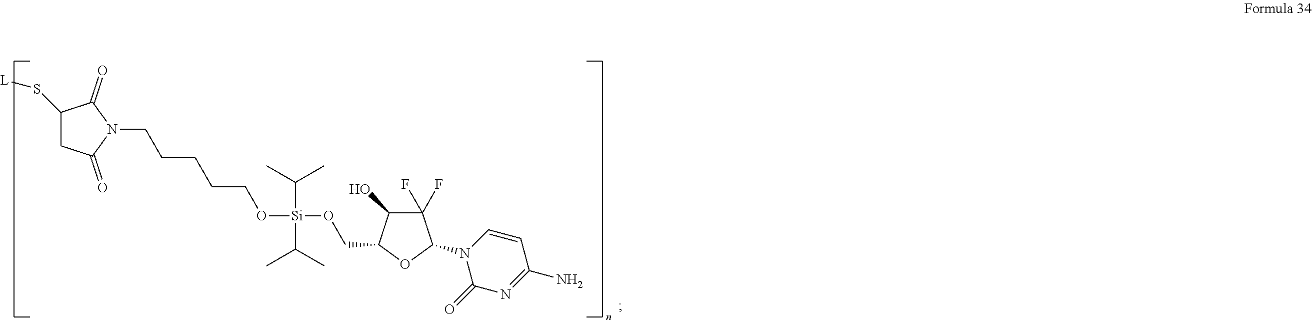

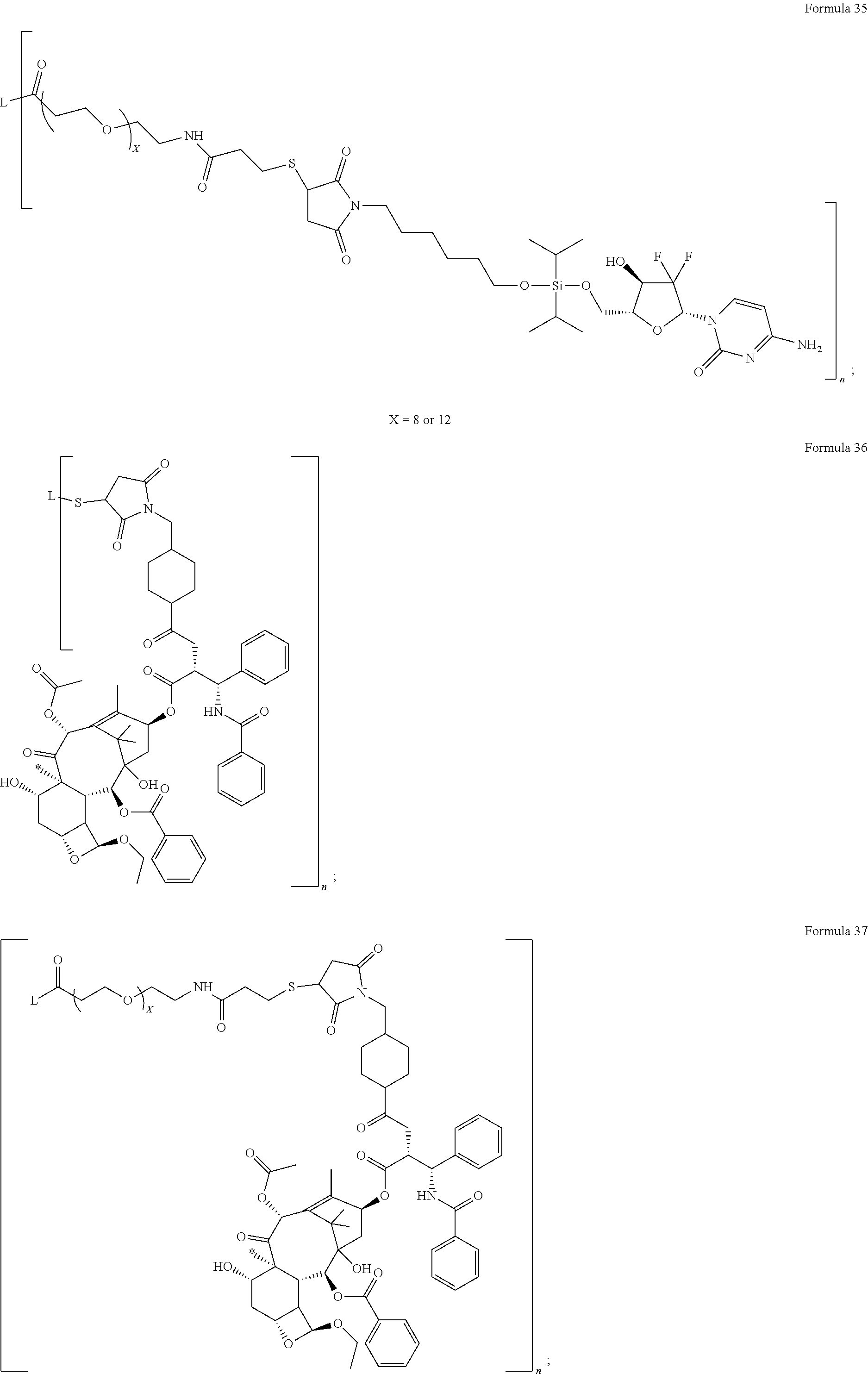

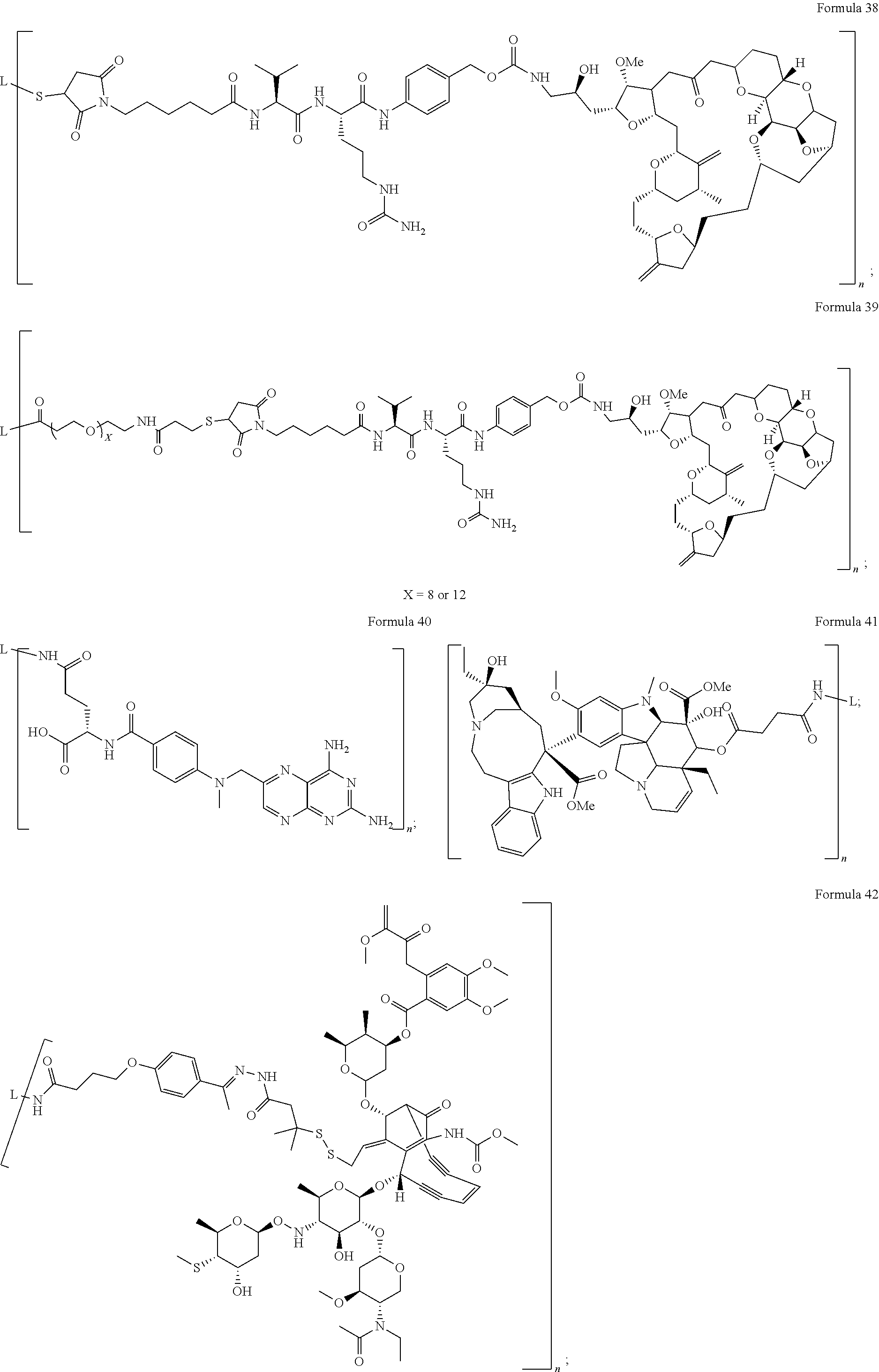

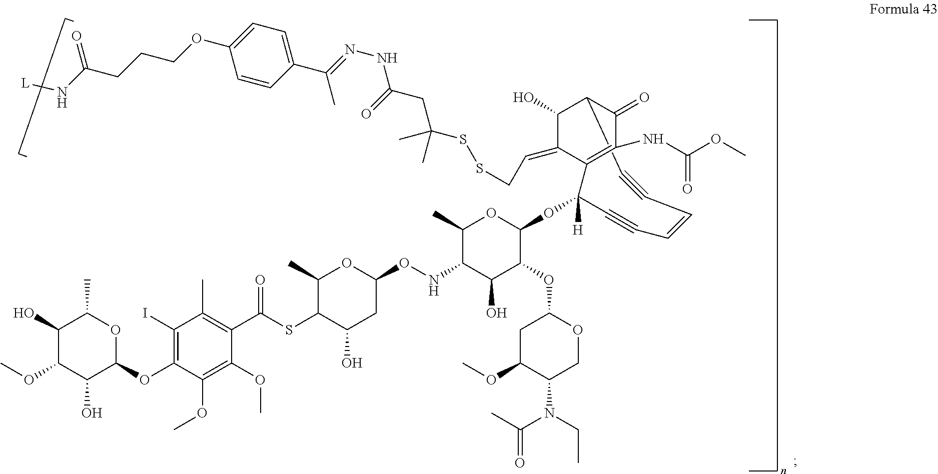

The inventive aspect of the present disclosure lies in the recombinant lectin conjugates of Formula I, II, III and IV. Though the linker, recombinant lectins and drugs used in the present disclosure are well known, the mere conjugation of the recombinant lectin either to drug directly or with aid of a random linker resulted in protein drug conjugate devoid of either solubility or of desired therapeutic effect.

The conjugates described herein have been designed to configure and position several components present in the Formula I, II, III, IV in such a way as to overcome not only one but both the solubility and therapeutic efficacy issues during the course of development. Surprisingly, the protein drug conjugate described herein also resulted in better stability along with improved solubility and therapeutic efficacy as compared to drug or lectin protein individually. Such designing, configuring and positioning of the several components that are part of the compounds of Formula I, II, III, or IV to form the protein drug conjugate with better efficacy, stability and solubility requires inventive step, which has not been taught in or motivated by the teachings of references cited above. Also, there is no teaching in the said references about selection of any specific lectin or drug or the linker which when conjugated would give compounds of the present invention and the desired therapeutic effects.

In an embodiment, the compounds of the present disclosure can be prepared by the methods known to the skilled person or by methods listed in ‘Methods to design and synthesize antibody-drug conjugates (ADCs). International journal of molecular sciences’ (by Yao H et al. volume 17, issue 2, page 194, 2016 or in ‘Preparation and characterization of antibody-drug conjugates acting on HER2-positive cancer cells’ (by Chiang Z-C et al, PLoS ONE, volume 15, issue 9, e02398132020). Exemplarily, the compounds can be prepared by reacting the lectin protein with the agent D using coupling reactions in presence of metal catalyst or by heating at feasible temperature or under pressure. As will be appreciated by the skilled person, the reaction of protein needs to be carried out under controlled conditions so that the protein or product comprising protein is not destabilized and denatured.

In an embodiment, the compounds of the present disclosure may be prepared by optionally reacting lectin protein with a moiety which is a part of a linker that may easily form a bond with the D or its pre-cursor or other part of the linker that is linked to D. Such reactions may be carried out at pH between 7 to 9 or sometimes higher or lower, in phosphate or carbonate-bicarbonate, or 4-(2-hydroxyethyl)-1-piperazineethanesulfonic acid (HEPES) or borate buffer or tris or glycine buffer. Such reactions may also be carried out at pH below 6 in buffers such as 4-morpholinoethanesulfonic acid (MES) or a carbodiimide reaction buffer. In an example, lectin protein may react with maleimide group at pH between 5 to 8, specifically in the absence of compounds or buffers containing thiol groups. In one particular embodiment of the present disclosure, the recombinant lectin for the preparation of compounds of present disclosure may be mixed with compound of Formula A at room temperature. The resulting compound of Formula B when subjected to reduction, gives compound of Formula C (Scheme I, wherein L is recombinant lectin selected from SEQ ID NO:2 or SEQ ID NO:5 and X ranges from 0-20).

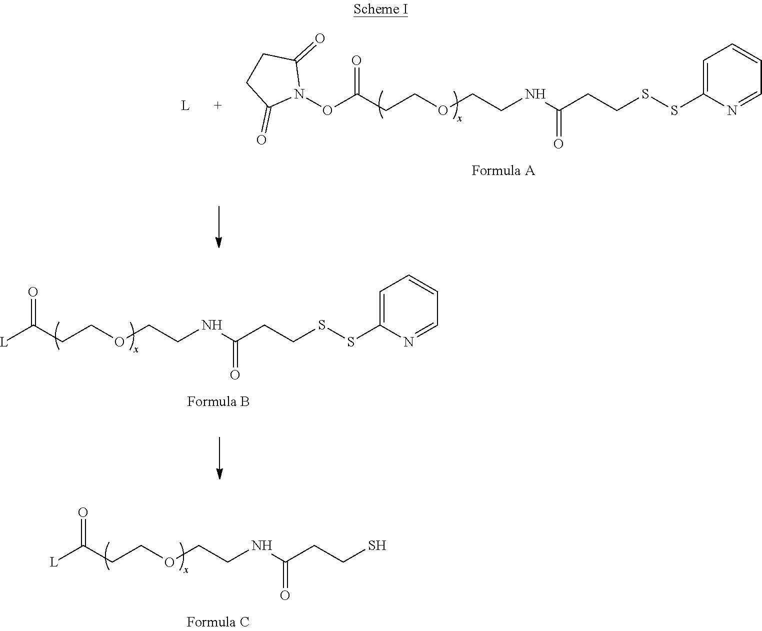

The lectin protein may be sometimes modified in certain un-conventional ways known to the skilled person. For example, addition of a non-natural amino acid to the protein and modifying it using Pictet-Spengler reaction or hydrazine-Pictet-Spengler reaction or other such variants or by using copper catalysed click reaction or variable click reaction such as click reactions in the absence of copper, or in presence or absence of any metal catalyst.

In an embodiment, the lectin protein or modified lectin protein is further reacted with cytotoxic agent or therapeutic agent or its precursor or other part of the linker that is linked to cytotoxic agent or therapeutic agent under conditions known to a person skilled in the art. In an example, such reaction may be carried out by reductive amination, or amidation, or thiol maleimide reaction. Such reactions may be carried out in buffers at a pH in the range from about 5 to about 9.

According to an embodiment, the final compound may be purified using known methods such as chemical purification or chromatographic purification for example, size exclusion chromatography (SEC) or hydrophobic interaction chromatography (HIC) or other high-performance liquid chromatographic techniques. The intermediates and final products may be characterized by methods such as Mass spectrometry, nuclear magnetic resonance (NMR) or any other method known to the skilled person. The quantitative distribution of cytotoxic agent or therapeutic agent to lectin protein can also be determined by electrophoresis among other methods described herein before.

According to an embodiment of the present disclosure, the compounds of present disclosure are highly pure. The term ‘highly pure’ may be construed as compound comprising at least 80% of single compound of present disclosure or at least 85% or at least 90% of compound of present disclosure. The word ‘single compound’ meaning there is present either of compound of Formula I, or II or III or IV, of which only single entity is present as single isomer or a single stereoisomer or polymorph or the solvate.

In an embodiment, the single compound means the mixture of compounds comprising different DAR values with same L, D and the linker. For example, compound of formula 15 with DAR values 1, 2 or 3 may be present as mixture and are considered as single compound for the purpose of the present disclosure. The compound of the present disclosure is said to be highly pure, if it comprises at least 80% of the mixture of products of same formula with different DAR values.

In another embodiment, a single compound is a compound of present disclosure with one DAR value. The compound is said to be highly pure if it comprises at least 80% of the compound with one DAR value and less than 20% of the compound comprises compound with other DAR values. For example, compound of Formula 15 is said to be highly pure if it comprises at least 80% of compound with DAR value 1 and the compound of Formula 15 with DAR 2 is present in less than 20%. Such compounds with same formula and different DAR values may be separated using methods known to the skilled person. For example, such compounds may be separated using size exclusion chromatography.

It would be appreciated by the skilled person that the purity of the compounds of the present disclosure is both in the solution form and the separated form. The compounds of the present disclosure may form aggregates in the solution. The pure compound of the present disclosure in solution comprises at least 80% of aggregates of compounds of single formula.

In fifth aspect of the present disclosure, there is provided a composition comprising compound of Formula I or Formula II or Formula III or Formula IV. The composition of the present disclosure may be formulated as an aqueous liquid or solid. In some embodiment the composition may be liquid, suspension, powder, sterile powder or lyophilized for solution formulation. Lyophilized formulation may be reconstituted with Water for Injection (WFI) and/or any suitable pharmaceutically acceptable diluent or mixture thereof to get required concentration as known by the skilled person. The composition is suitable for single dose or multiple doses. A person skilled in the art knows that the type of dosing is dependent on various factors, such as body height and weight, body surface area, age, gender, or general health of the patient, and on the preparation to be administered in particular, the duration and type of administration, and on other medications that may be administered in parallel.

The compositions of the present disclosure may be administered to an individual in a suitable dosage. The administration can take place locally, enterally, or parenterally, for example, intravenously, intraperitoneally, subcutaneously, intramuscularly, locally, intranasally, intrabronchially or intradermally, or via a catheter at a point in an artery. In a particular embodiment, the compositions of present disclosures may be administered parenterally.

According to some embodiments, the effective concentration of compound of Formula I or Formula II or Formula III or Formula IV is in the range from 0.05 μg/mL to 100 μg/mL. In a particular embodiment, the effective concentration is in the range from 0.05 μg/mL to 50 μg/mL. According to some embodiments, the effective concentration of the cytotoxic drug or therapeutic agent is in the range from 0.001 nM to 1 μM.

In an aspect, the composition of the present disclosure may further comprise pharmaceutically acceptable excipients which may be selected from a buffer, a stabilizer, a polymers, a solubilizer, cryoprotectants, lyoprotectants, bulking agents, diluents emulsifiers and preservatives.

According to an aspect, the excipients may include polymer such as polyethylene glycols (PEGs), dextran, hydroxyl ethyl starch (HETA) or combination thereof, and a protein such as human serum albumin or Gelatin or combination thereof.

According to another aspect the stabilizer may be selected from surfactants, detergents, amino acids, pharmaceutically acceptable salt of amino acid, carbohydrates or sugar stabilizers, amines, polyols or combination thereof.

The liquid formulation of the present invention may preferably comprise buffer. The buffer for example may be selected from phosphate buffer such as sodium monohydrogen phosphate dehydrate, sodium dihydrogen phosphate dehydrate, phosphate buffer such as dibasic potassium phosphate, monobasic potassium phosphate citrate buffer, acetate buffer, TRIS-sodium chloride buffer, or histidine. The concentration of the buffer may range from 1 mM to 300 mM. As understood by the skilled person, pH of the formulation may be regulated between 5 and 9.

According to a specific aspect of the present disclosure, there is provided a composition comprising compound of formula I, formula II, formula III or formula IV and TRIS-sodium chloride buffer.

The composition of the present disclosure may be formulated using the standard methods known to the skilled person or by the methods disclosed in “Protein formulation and Delivery” by E. J. McNally et al, or in “Rational Design of Stable Protein Formulations Theory and Practice” by J. F. Carpenter et al.

In one embodiment, the compounds of present disclosure has application in the prevention of growth or metastasis of tumor, treatment or to cure cancer. According to some embodiments, the compound of Formula I or Formula II or Formula III or Formula IV may be used for prevention or treatment of adenocarcinoma, Squamous cell carcinoma, Transitional cell carcinoma, Basal cell carcinoma, Sarcomas, Lymphomas, epithelial cell carcinomas or non-epithelial cell carcinomas. According to the particular embodiment, therapeutically effective compound of Formula I or Formula II or Formula III or Formula IV of the present disclosure may be used for prevention or treatment of pancreatic cancer, abdominal cancer, liver cancer, prostate cancer, oral cancer, colon cancer, ovary cancer, bladder cancer, kidney cancer, stomach cancer, breast cancer, bone marrow cancer, melanoma, leukemia or central nervous system cancer in a subject.

In sixth aspect of the present disclosure, there is provided a method of treatment or prevention of cancer using compound of Formula I or Formula II or Formula III or Formula IV. The method of treatment or prevention may comprise administering a therapeutically effective amount of the said compound to the subject. In some embodiments, the compound of present disclosure is administered at a dose of from 0.01 to 1000 mg/kg, from 0.05 to 100 mg/kg or from 0.1 to 50 mg/kg. It will be within the capabilities of the skilled person to determine an amount of compound of Formula I or Formula II or Formula III or Formula IV to be administered according to the nature of the condition being treated and the subject.

In one embodiment of the present disclosure the compounds of present disclosure such as compounds of Formula 15 to Formula 19 were subjected to cytotoxicity assay against the bladder cancer T24 cell lines by determining the IC₅₀ values. The compound of Formula 15 showed IC₅₀ value of 41.13 nM, whereas compound of Formula 17 showed IC₅₀ value of 4.216 nM, Formula 16 showed 325.4 nM, compound of Formula 18 showed IC₅₀ of 19.93 nM and compound of Formula 19 showed IC₅₀ valued of 70.64 nM. Similarly, in an cytotoxicity assay against the ovarian cancer PA-1 cell lines compound of formula 15 showed IC₅₀ value of 40.9 nM, Formula 16 showed IC₅₀ value of 17.7 nM, compound of formula 17 showed 2.61 nM, Formula 18 showed 1.04 nM and compound of formula 19 showed 5.22 nM.

MMAE individually indicated IC₅₀ value of 3.23 nM and mc-vc-PAB-MMAE, indicated 646.9 nM whereas Tesirine indicated 13.42 nM against PA-1 cell line. On the other end MMAE individually indicated IC50 value of 0.24 nM whereas Tesirine indicated 144.8 nM against T24 cell line.

Abbreviations:

| | |
|---|---|
| DMA | Dimethylacetamide |
| DMSO | Dimethyl sulfoxide |
| DNA | Deoxyribonucleic acid |
| DO | Dissolved Oxygen |
| DTPA | Diethylenetriamine pentaacetate |
| DTT | Dithiothreitol |
| E. coli | Escherichia coli |
| EDTA | Ethylenediamine tetraacetic acid |
| EMEM | Eagle's Minimum Essential Medium |
| FBS | FBS |
| FDA | Food and Drug Administration |
| HCl | Hydrochloric acid |
| HIC | Hydrophobic interaction chromatography |
| HPLC | High performance Liquid Chromatography |
| IgG | Immunoglobulin G |
| IPTG | Isopropyl β-D-1-thiogalactopyranoside |
| K₂HPO₄ | Dipotassium hydrogen phosphate |
| KDa | Kilo Dalton |
| LCMS | Liquid Chromatography Mass spectrometry |
| MgSO₄•7H₂O | Magnesium sulfate heptahydrate |
| MTS- PMS | MTS [3-(4,5-dimethylthiazol-2-yl)-5-(3- |
| | carboxymethoxyphenyl)-2-(4-sulfophenyl)-2H- |
| | tetrazolium inner salt and PMS an electron |
| | coupling reagent (phenazine methosulfate). |
| Na₂HPO₄ | Disodium phosphate |
| NaCl | Sodium chloride |
| NEM | N-Ethylmaleimide |
| (NH4)₂SO₄ | Ammonium sulfate |
| OD | Optical Density |
| PBS | Phosphate Buffer Saline |
| SEC | Size exclusion Chromatography |
| TBS | Tris-Buffered Saline |
| TFF | Tangential Flow Filtration |
| v/v | Volume/volume |
| vvm | Volume of liquid per minute |
| w/v | weight/volume |
| PDC | Protein Drug Conjugate |

### EXAMPLES

Various aspects of the disclosure are further described by way of below listed examples. The examples are illustrious, and the skilled person is well aware and informed about the obvious variations of them. The examples therefore do not limit the scope of this disclosure in any manner.

### Example 1: Clone and Manufacturing Process of Recombinant Lection Having Amino Acid Sequence of SEQ ID NO:2

The nucleotide sequence (SEQ ID NO: 6) coding for the recombinant lectin having amino acid sequence of SEQ ID NO:2 cloned into pET27b vector (sourced from Novagen) using NdeI and BamHI to generate pET27b-Lec construct. Further the pET27b-Lec construct was transformed into propagation host E. coli Top10 competent cells. Plasmid was isolated form transformed cells and insert integrity was confirmed by restriction digestion analysis and the gene sequence is confirmed by DNA sequencing. Verified pET27b-Lec plasmid was further transformed into E. coli BL21 DE3 (Gold) expression host. Transformed clones were tested for recombinant protein expression and the clone showing high expression is verified by DNA sequencing and further used for cell bank preparation. Characterized cell bank is used in fermentation process.

For fermentation, seed culture was prepared by inoculating the culture from glycerol stock in medium containing 2% Hiveg Luria broth, 0.75% Na₂HPO₄, 0.5% dextrose and kanamycin (20 μg/ml). The culture was grown at 30±2° C. and 110 rpm for 16 hours. Approximately 300 m1 culture was inoculated into 2.3 L of production medium containing. 1% yeast extract (w/v), 1.2% Dextrose (w/v), 0.3% KH₂PO₄ (w/v), 1.25% K₂HIPO₄ (w/v), 0.5% (NH₄)₂SO₄ (w/v), 0.05% NaCl (w/v), 0.1% MgSO₄·7H₂O (w/v), 0.1% (v/v) trace metal solution, kanamycin (20μ/ml). Fermentation was carried out in 5 L fermenter (BIOSTAT B, Sartorius Stedim) with 1-2 vvm aeration, dissolved oxygen was maintained at 50%-60% and pH maintained was maintained at 6.6-7.0 with alkali. Initial growth was carried out at a temperature of 37° C. Temperature was gradually decreased and maintained at 22° C. during the induction phase. Feeding of Carbon (glycerol) and Nitrogen source (yeast extract) was initiated after 5 log hours at predetermined feeding rates, maintaining C:N ratio in the range of 4:1. Culture was induced at cell density of ˜45 (OD600) with 1 mM IPTG. Fermentation was continued till 24 hrs and culture broth was harvested by centrifugation at 9000 rpm for 15 minutes. The wet weight of cell mass obtained from fermentation broth was approximately 350 g.

The cell pellet was suspended in lysis buffer (25 mM tris, 1 mM EDTA, pH 8.5) in a ratio of 1:10 (w/v) and stirred for at least 2 hr on an overhead stirrer to form a homogenous suspension. The suspension was lysed by high pressure homogenization at ˜18,000 psi. The cell lysate was centrifuged at 9,000 rpm for 15 min at 15° C. The resultant supernatant was retained and processed for further clarification by treating with at least 0.025% polyethyleimine by stirring for 15-30 mins. Treated cell lysate was clarified using a 0.1p m hollow fiber of 3600 cm2 area pre equilibrated with 25 mM Tris buffer containing 1 mM EDTA, pH 8.0±0.5. Transmembrane pressure of 5-10 psi was maintained throughout the clarification process.

The recombinant lectin was purified using four step chromatography. In the first step cell lysate was loaded on Cellufine Max Q-r resin pre equilibrated with 25 mM Tris buffer containing 1 mM EDTA, pH 8.0±0.5 and washed with the equilibration buffer containing 1-3 g/L NaCl. Protein with 77.4% purity was eluted with 25 mM Tris buffer containing 1 mM EDTA, 11-15 g/L NaCl, pH 8±0.5. In second step the pH of the column 1 elute was adjusted to 4.5 with acetic acid followed by ammonium sulphate precipitation. After centrifugation clear supernatant was processed on the HIC resin (Cellufine Max Butyl) equilibrated with 25 mM sodium acetate buffer containing 1 mM EDTA, 0.5-2M ammonium sulphate, pH 4.5. After washing with equilibration buffer, the protein with purity of 90.3% was eluted with 25 mM Sodium acetate, 1 mM EDTA, 15 g/L ammonium sulphate, pH 4.5. In third step the protein is purified by cation exchange chromatography, where the column 2 elute was diluted to approximately 20 mS/cm conductivity with purified water and loaded onto SP Sepharose FF resin pre equilibrated with 25 mM Sodium acetate buffer containing 1 mM EDTA, pH 4.5. After washing with equilibration buffer, the protein with purity of 93.0% was eluted by a step gradient of 70% using elution buffer containing 25 mM Sodium acetate, 1 mM EDTA, 0.5 M NaCl, pH 4.5. In fourth step the column 3 elute was buffer exchanged with 25 mM Tris buffer, pH 8.0 using 3 KDa membrane and processed onto Source 30Q anion exchange resin, pre equilibrated with 25 mM Tris buffer, pH 8.0. After washing the elution was carried out by linear gradient using elution buffer containing 25 mM Tris, 0.5 M NaCl, pH 8.0 for 15 column volumes. The fractions having purity of >99.0% by HPLC analysis were pooled and total of 9.0 grams of protein was obtained as measured by optical density at 280 nm considering 1OD at 280 nm equals 1 mg. The final elute fractions were buffer exchanged against TBS Buffer (50 mM Tris buffer, 150 mM NaCl, pH 7.8).

### Example 2: Clone and Manufacturing Process of Recombinant Lection Having Amino Acid Sequence of SEQ ID NO: 5

The nucleotide sequence (SEQ ID NO: 7) coding for the recombinant lectin having amino acid sequence of SEQ ID NO: 5 cloned into pET27b vector using NdeI and BamHI to generate pET27b construct. Further the pET27b construct was transformed into propagation host E. coli Top10 competent cells. Plasmid was isolated form transformed cells and insert integrity was confirmed by restriction digestion analysis and the gene sequence is confirmed by DNA sequencing. Verified pET27b plasmid was further transformed into E. coli BL21 DE3 (Gold) expression host. Transformed clones were tested for recombinant protein expression and the clone showing high expression is verified by DNA sequencing and further used for cell bank preparation. Characterized cell bank is used in fermentation process.

For fermentation seed culture was prepared by inoculating the culture from glycerol stock in medium containing yeast extract (10 g/L), potassium dihydrogen phosphate (3 g/L), di-potassium hydrogen phosphate (12.54 g/L), ammonium sulphate (5 g/L), sodium chloride (0.5 g/L), dextrose (12 g/L), magnesium sulphate heptahydrate (1 g/L), kanamycin sulphate (20 mg/L) and trace metal solution (1 ml/L). The fermentation was initiated at 30° C. and later the temperature was gradually decreased to 18° C. DO level was maintained at 60% and pH was maintained at 6.80±0.05 with alkali solution except during carbon source shifting. Fed batch fermentation process was used with glycerol (50%) and yeast extract (40%) as carbon and nitrogen source respectively as feed medium. Induction was done with 0.15 mM IPTG at OD600 nm around 60. Total batch run time was 48 to 72 hours and maximum OD600 achieved was in the range of 107 after 52 log hours. Expression of the recombinant protein having amino acid sequence of SEQ ID NO 5 was around 6.3 g/L. Fermentation broth was harvested from the fermenter. It is then centrifuged at 9000 rpm, for 10-15 minutes at 10° C. to obtain compact cell pellet. Pellet obtained is then suspended in 1:10 ratio (w/v) of lysis buffer containing 25 mM Tris HCl, 1 mM EDTA, pH 8.5 and suspension is stirred for 1-2 hours using mechanical stirrer maintaining a temperature of 10±4° C. Lysis is carried out using high pressure homogenizer at 1100±200 bar by giving 1-2 passes. The cell lysis efficiency of >90-95% is obtained (determined by OD600) and lysate collect at low temperature.

The cell lysate was clarified using 0.1μ TFF and permeate was collected. In order to recover maximum quantity of protein, the retentate was washed 4-6 times with 25 mM Tris-HCl buffer, 1 mM EDTA, pH 8.0 in step mode till protein absorbance at 280 nm came down to less than 4-6. The solution was later clarified using 0.1p hollow fiber filter with Trans Membrane Pressure of 2-10 psi. The washes that contained the protein were pooled with the main permeate. Temperature was maintained below 25° C. during entire clarification process.

The recombinant lectin of SEQ ID NO:5 was purified using three step chromatography. In first step cell lysate was loaded on Cellufine Max Q-r resin pre equilibrated with 25 mM Tris buffer containing 1 mM EDTA, pH 8.0±0.5 and washed with the 25 mM Tris HCl, 1 mM EDTA, 50 mM NaCl at pH 8.0. Protein was eluted with 25 mM Tris HCl, 1 mM EDTA, 200 mM NaCl at pH 8.0. Entire peak was collected as a single fraction. In second step the recombinant lectin of SEQ ID NO:5 eluted from first step was bound to the CM Sepharose column pre equilibrated with the equilibration buffer at a binding capacity of 20 mg/ml. Post complete loading, column was washed with the equilibration buffer 25 mM Sodium Acetate, 5 mM β-Mercaptoethanol, pH 5.1 to remove the unbound protein. Elution was carried out giving a linear gradient from 0-50% 25 mM Sodium Acetate, 1.0M NaCl, 5 mM β-Mercaptoethanol, pH 5.1 for 15 column volumes followed by a wash of 100% buffer 25 mM sodium scetate, 1.0M NaCl, 5 mM β-Mercaptoethanol, pH 5.1. Elute obtained from CM Sepharose column then subjected to pH adjustment to 8.0 with Tris buffer and buffer exchanged against 25 mM Tris, 5 mM Beta mercaptoethanol, pH 8.0 to get conductivity to less than 2.0 mS/cm using 5 KDa membrane. Then recombinant lectin of Seq ID: 5 was loaded in third column Source 30Q pre-equilibrated with equilibration buffer: 25 mM Tris, 5 mM Beta mercaptoethanol, pH 8.0. The recombinant protein of Seq ID No:5 was then eluted using elution buffer of 25 mM Tris, 5 mM Beta mercaptoethanol, 500 mM NaCl, pH 8.0. The recombinant protein of Seq ID No: 5 with purity in excess of 95% was obtained.

### Example 3: General Preparation of Compound of Formula C

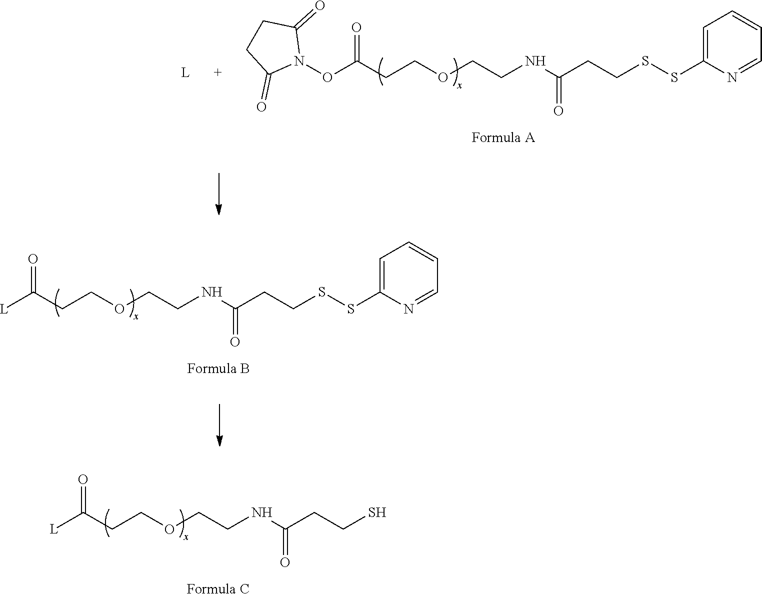

Wherein
L− Recombinant lectin of SEQ ID NO: 2
X− 8 or 12

A solution of recombinant lectin of SEQ ID NO: 2 12.2 mg/L in 1×PBS was diluted with a 1:1 volume of 100 mM sodium phosphate dibasic, 150 mM NaCl and 10 mM DTPA, pH 8.0. To this was added 2.4 equivalents (with respect to SEQ ID NO: 2) of commercially available Formula A (10 mM DMSO) purchased from Quanta Biodesign Limited. The reaction was incubated with gentle mixing for 2 hours and the degree of conjugation was determined by LCMS. If Drug to protein ratio (DAR) was less than 2, another equivalent of 10 mM Formula A was added and incubated another hour at room temperature. After incubation and confirmation of formation of Formula B with DAR of more than 2 by LCMS analysis, DTT was added to the reaction mixture to achieve a final concentration of 50 mM and incubated at 37° C. for 30 minutes in order to obtain a compound of Formula C, which was further purified using Superdex 75 SEC column in 1×PBS, 10 mM EDTA to generate purified compound of Formula C with which gave a final concentration of 4.8 mg/mL in 1×PBS, 10 mM EDTA.

### Example 4: Preparation of Conjugate of Formula 15

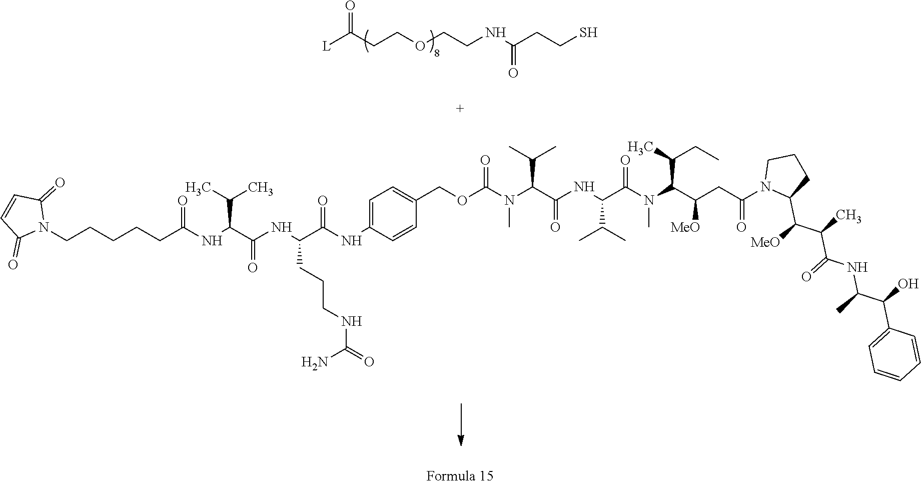

### L− Recombinant Lectin of SEQ ID NO 2

45 mg of a solution (4.8 mg/mL) of Compound of formula C in 1×PBS and 10 mM EDTA was further diluted with to final concentration 2.5 mg/m1 by 1×PBS with 10 mM EDTA, DMSO and 1.5 eq of 10 mM MC-VC-PAB-MMAE in DMSO containing 6% of cyclodextrin that resulted in a final DMSO concentration of 35% (v/v). The solution was stirred at room temperature for 60 min followed by capping with excess 10 eq NEM. The drug to protein ratio (DAR) of the conjugate Formula 15 was determined by LCMS analysis. Further the conjugate of Formula 15 was subjected to SEC using GE10-300 Superdex 75 column) with buffer exchange to 50 mM Tris, 150 mM NaCl and pH was adjusted to 8.0 using tris buffered saline TBS. Post SEC purification, DAR value of conjugate of Formula 15 was determined with the aid of LCMS. The DAR value was determined to be 1.09.

### Example 5: Preparation of Conjugate of Formula 17

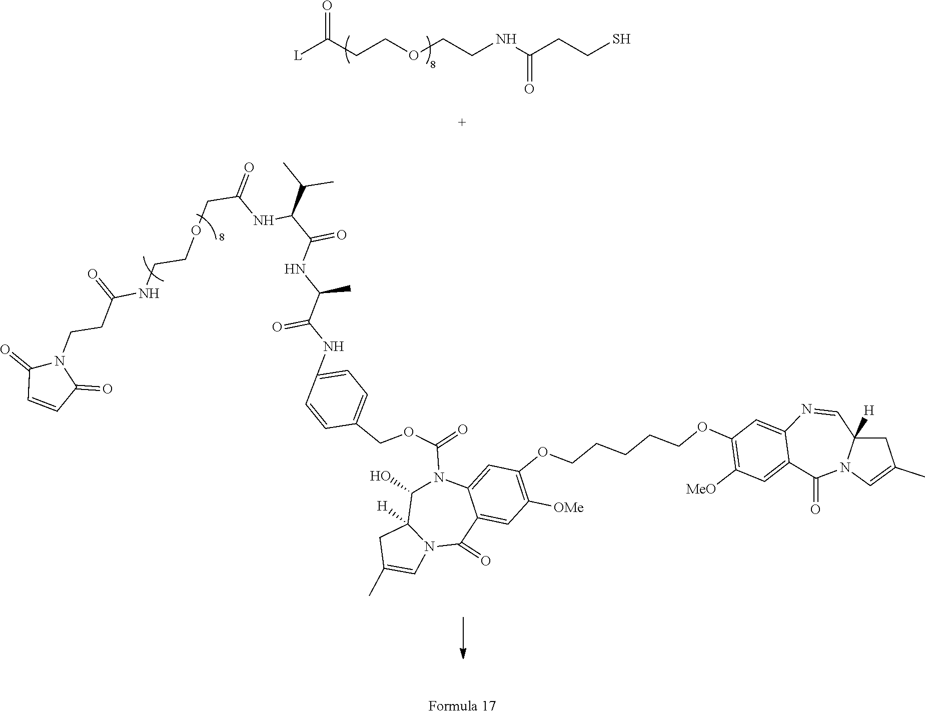

### L− Recombinant Lectin of SEQ ID NO 2

34 mg of a solution (4.8 mg/mL) of Formula C in 1×PBS and 10 mM EDTA was further diluted with to final concentration 2.5 mg/m1 by 1×PBS with 10 mM EDTA, DMSO and 2.5 eq of 10 mM Tesirine in DMSO that resulted in a final DMSO concentration of 35% (V/V). The solution was stirred at room temperature for 60 min followed by capping with excess 10 eq NEM. The drug to protein ratio DAR of conjugate Formula 17 formed was determined using LCMS analysis. The final product of Conjugate of Formula 17 was subjected to SEC using GE10-300 Superdex 75 column with buffer exchange to 50 mM Tris, 150 mM NaCl and pH was adjusted to 8.0 using TBS. Post SEC purification DAR of Conjugate of Formula 17 was determined using reverse phase liquid chromatography and was found to be 1.25.

### Example 6: Preparation of Conjugate of Formula 19

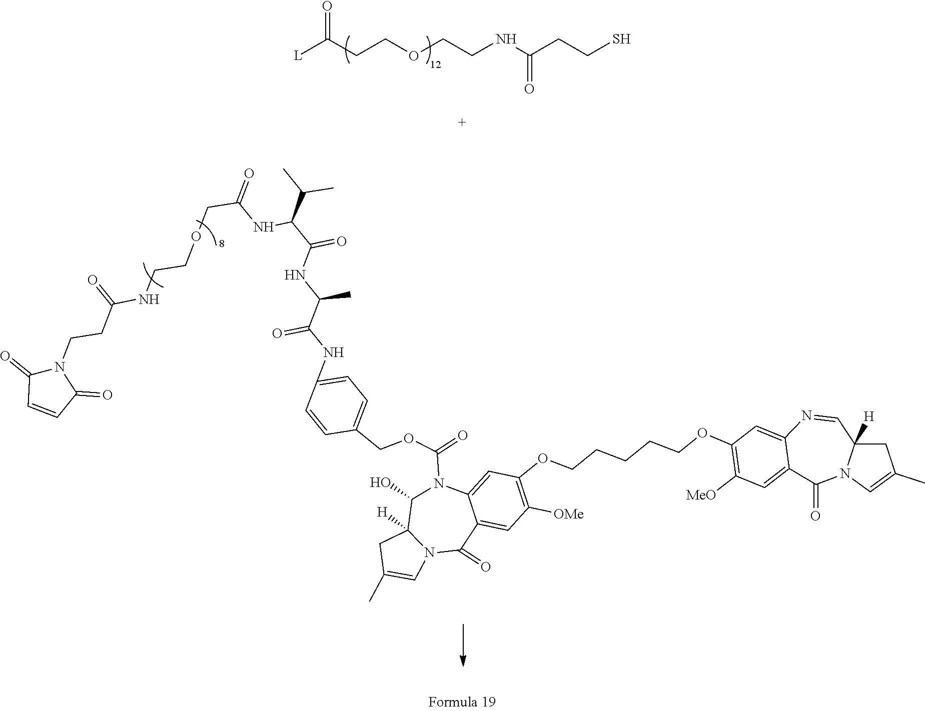

### L− Recombinant Lectin of SEQ ID NO 2

10.3 mg of Formula C and 10 mM EDTA was further diluted with to final concentration 2.5 mg/m1 by 1×PBS with 10 mM EDTA, DMSO and 2.5 eq of 10 mM Tesirine in DMSO that resulted in a final DMSO concentration of 35% (v/v). The solution was stirred at room temperature for 60 min followed by capping with excess 10 eq NEM. Then the drug to protein ratio DAR of conjugate of Formula 19 was determined by LCMS analysis. The Conjugate of Formula 19 was subjected to SEC using GE10-300 Superdex 75 column with buffer exchange to 50 mM Tris, 150 mM NaCl and pH was adjusted to 8.0 using TBS. Post SEC purification DAR value of conjugate of Formula 19 was determined to be 1.32.

### Example 7. Preparation of Conjugate of Formula 16 Wherein the L is Linked Via a Cystine (SH) of to the Linker Drug Complex and the Linkage is Directly to the Protein Cysteine SH

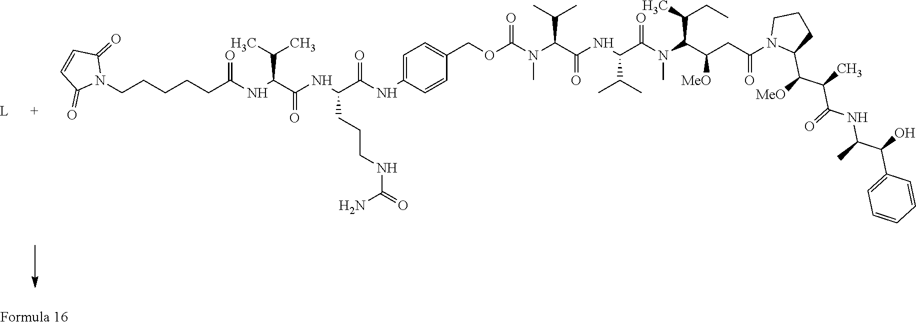

### L− Recombinant Lectin of SEQ ID NO 5

30 mg of recombinant lectin of Seq ID No 5 in 1×PBS (7.0 mg/ml) was further diluted with to final concentration 2.5 mg/m1 by 1×PBS with 10 mM EDTA, DMA and 3.0 eq of 10 mM MC-Val-Cit-PAB-MMAE in DMA that resulted in a final DMA concentration of 20% (V/V). After 2 hours the reaction was incomplete and another 3.0 eq of MC-Val-Cit-PAB-MMAE was added and incubated for 2 h. Then the conjugate of Formula 16 was subjected to LCMS analysis in-order to determine the DAR value. The product formed was subjected to size exclusion chromatography (SEC) using GE10-300 Superdex 75 column with buffer exchange to 50 mM Tris, 150 mM NaCl and pH was adjusted to 8.0 using TBS. Post SEC purification DAR value of conjugate of Formula 16 was determined to be 1.0.

### Example 8. Preparation of Conjugate of Formula 18

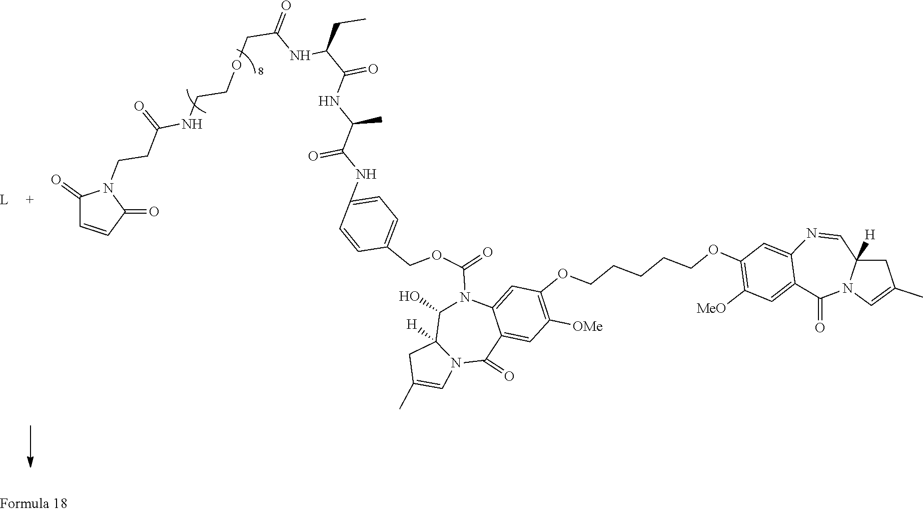

### L− Recombinant Lectin of SEQ ID NO 5

5 mg of recombinant lectin of Seq ID No 5 in 1×PBS (7.0 mg/ml) was further diluted to final concentration 2.5 mg/ml by 1×PBS with 10 mM EDTA, DMA and 3.0 eq of 10 mM Tesirine in DMA that resulted in a final DMA concentration of 20% (v/v). After 2 hours the reaction was incomplete and another 3.0 eq of Tesirine was added and incubated for 2 h. Then the conjugate Formula 18 was subjected to LCMS analysis in-order to determine the DAR value. The product formed was subjected to SEC using GE10-300 Superdex 75 column with buffer exchange to 50 mM Tris, 150 mM NaCl and pH was adjusted to 8.0 using TBS. Post SEC purification DAR value of conjugate of Formula 18 was determined to be 1.05.

### Example 9: Bioassay—Cytotoxicity Effect of Conjugate of Formula 15, 17 and 19 Against Ovarian Cancer Cell Line (PA-1) and Human Urinary Bladder Cancer Cell Line (T-24)

T-24 Cell line were cultured in McCoy's 5a Medium Modified supplemented with 10% FBS and the PA-1 Cell line was cultured in EMEM Media supplemented with 10% FBS. The assay was performed by plating 5,000 cells/well and incubated at 37° C. overnight. Recombinant lectin protein of SEQ ID NO: 2 conjugates of formula 15, 17 and 19 were made in respective media and diluted 10× starting from 10 micromolar concentration for 11 points of concentration. Each concentration was added in triplicate and media-only wells used as a control. After incubation for 72 h cell titer glow (Promega) reagent (50 μL) was added to each well and the plates shaken for 5 mins and the luminescence was recorded. Percent viability was determined by dividing luminescence of the treated well by the non-treated well and multiplying by 100. Data was analysed using non-linear regression with a logarithmic scale.

TABLE 1
Cytotoxicity effect of Conjugate of Formula 15, 17
and 19 against ovarian cancer cell line (PA-1) and
human urinary Bladder cancer cell line (T-24)

| | IC₅₀ values | | |
|---|---|---|---|
| compound | | PA-1 Cell line | T-24 cell line |

| | | | | |
|---|---|---|---|---|
| Formula 15 | 40.9 | nM | 41.13 | nM |
| Formula 17 | 2.61 | nM | 4.216 | nM |
| Formula 19 | 5.225 | nM | 70.64 | nM |
| SEQ ID No: 2 | 20.3 | μg/ml | 10.4 | μg/ml |

### Conclusion

SEQ ID No. 2 does not exhibit major cytotoxic effects below 10 μM concentration in both cell lines. IC50 values are in the rage of 15-25 uM for SEQ ID No. 2, whereas for Formula 15, 17 and 19; IC50 values are in nanomoles i.e., conjugates displayed potent activity against PA-1 cell lines and T-24 cell lines.

### Example 10: Bioassay—Cytotoxicity Effect Conjugate of Formula 16 and 18 Against Ovarian Cancer Cell Line (PA-1) and Human Urinary Bladder Cancer Cell Line (T-24)

5,000 cells/well were plated and incubated at 37° C. overnight. Conjugates/vehicle control dilutions were made in respective media and added to cells. Volume added: 50 uL to each well. Starting treatment concentration was 1 uM for Conjugate of formula 16 and 18, and control SEQ ID NO: 5 alone and 10% for vehicle control (1×PBS with 20% DMA) and then diluted 10-fold down for a total of 11 treatment dilutions. Each concentration was analysed in triplicates. Media-only wells was used as a control to calculate percent viability. Cell titer glow reagent (volume: 50 uL) was added to each well, the plate was shaken for 5 mins and the luminescence was recorded. Drug Treatment Time: 72 hours. Percent viability was calculated by dividing the luminescence signal obtained for each treated well by the untreated well (media-only control) and multiplying by 100.

$$Data was next transformed using X = Log ( x ) and then analysed with$$

$$nonlinear regression ( curve fit ) , Dose Response inhibition - log$$

$$( inhibitor ) vs response ( 3 parameters ) using PRISM software to$$

$$determine the IC 50 value .$$

TABLE 2
Cytotoxicity effect of Conjugate of Formula 16 and
18 against ovarian cancer cell line (PA-1) and
human urinary Bladder cancer cell line (T-24):

| | IC50 values | | |
|---|---|---|---|
| Compound | | PA-1 Cell line | T-24 cell line |

| | | | |
|---|---|---|---|
| Formula 16 | 17.7 | nM | 325.4 nM |
| Formula 18 | 1.04 | nM | 19.93 nM |
| SEQ ID NO:5 | 16 | μg/ml | — |

### Conclusion

SEQ ID No. 5 does not exhibit major cytotoxic effects below 10 uM concentration in both cell lines.

Seq ID NO. 5 IC50 values are in the rage of 15-25 uM; whereas for Formula 16 and 18, IC50 values are in nanomoles i.e., conjugates displayed potent activity against PA-1 cell lines and T-24 cell lines.

### Example 11: Glycan Binding Study and Co-Localization Study

### A. Glycan Binding Study was Performed to Understand the Presence and Specificity of the Lectin Having SEQ ID NO 2 TowArds Glycans Expressed on Cancer Cells.

Lectin of Seq ID No 2 was assayed with following glycan arrays for identifying specific binding motifs:
O-Glycan array containing 94 glycans; and
N-Glycan array containing 100 glycans

### Observations:

The said Lectin having Seq ID No 2 showed specific binding to N-glycans containing GlcNac at the non-reducing end; estimated Kd with terminal N-glycan GlcNAc motifs were between 0.238 to 0.460 Kd (ug/ml).
Lectin having Seq ID No 2 showed strong and broad binding profile to O-glycans, specifically to T-antigen and its extended core structures. Main binding motifs found were O-GalNAc Core1 (T antigen); Extended Core1 structures; Core2; α2,3/6-sialyl Core1; and α2,6/6-sialyl Core2. Estimated Kd for T antigen is 0.319, Sialyl T antigen is 0.670 to 1.756 and for core 2 is 0.212 Kd (ug/m).

### B. Colocalization Study of SEQ ID NO 2

Study was performed to analyze the localization of SEQ ID NO 2 (test item) in cell organelles using Confocal microscopy.

Test Item provided as aqueous solution was tagged with FITC label at Bioklone Biotech Pvt Ltd. Resultant concentration of FITC tagged test Item was 1 mg/mL.

The stock solution of Test Item was diluted in Serum Free Medium (SFM) at single concentration—100 μg/mL.

Cells were plated on a glass coverslip at a density of 0.1×10⁶ cells per well. After complete adhesion of the cells on the coverslip, cells were treated with 100 g/mL of FITC-Recombinant Lectin having amino acid sequence of SEQ ID NO: 2 for 2 hours at 37° C.

Following the incubation cells were washed with PBS (3 washes) and fixed using 4% paraformaldehyde for 10 min at RT. After fixation, cells were permeabilized using 0.5% IGEPAL for 10 min at RT (Permeabilization was not done for cells that were stained with Pan-Cadherin). Post permeabilization, cells were blocked using 5% normal goat serum for 1 hour at RT. Post blocking, cells were treated with organelle specific markers for 1.5 hours at RT followed by secondary antibody for 2 hours at RT.

Following antibodies were used for different organelles:

TABLE 3
Antibodies used for organelles

| | Organelle | | | |
|---|---|---|---|---|
| | Specific | | Secondary | |
| Organelle | marker | Dilution | antibody | Dilution |
| Mitochondria | MTCO2 | 1: 50 | Texas red | 1:500 |
| | | | Goat Anti- | |
| | | | mouse IgG | |
| Endoplasmic | Calnexin | 1: 50 | Anti-mouse | 1:500 |
| reticulum | | | Texas red | |
| Golgi bodies | GM130 | 1: 50 | Anti-mouse | 1:500 |
| | | | Texas red | |
| Nuclear | Lamin | 1: 50 | Anti-mouse | 1:500 |
| Envelope | | | Texas red | |
| Plasma | Pan | 1: 50 | Anti-rabbit | 1:500 |
| Membrane | Cadherin | | alexa fluor | |
| | | | red | |

Post incubation, cells were washed using PBST (3 washes) and PBS (3 washes) and stained with DAPI (nucleus stain) for 3 min at RT.

Cells were washed (3 washes) and mounted on a glass slide using Prolong Glass antifade mountant. Imaging was done using Confocal fluorescence microscope (Make: Olympus FV1000) at AIRF, JNU. Images were captured using software FluoView.

To determine the co-localization of Recombinant Lectin having amino acid sequence of SEQ ID NO:2 (Recombinant Protein), bladder cancer cells (T24) were incubated with Recombinant Lectin having amino acid sequence of SEQ ID NO:2 labelled with FITC tag for 2 h. This was followed by staining with organelle specific markers-MTCO2 (Mitochondria), Calnexin (Endoplasmic reticulum), GM130 (Golgi bodies), Pan Cadherin (Plasma membrane) and Lamin A/C (Nuclear Membrane). Cells were imaged using confocal microscope.

Results demonstrated that Recombinant Lectin having amino acid sequence of SEQ ID NO:2 (Recombinant Protein) (Recombinant Protein) colocalized in Mitochondria, Endoplasmic reticulum, Golgi bodies and Plasma membrane (red fluorescence). However, no localization was observed in the nuclear envelope. [Refer FIG. 6].

### Example 12: Cytotoxic Effect of Conjugates were Evaluated in a Panel of Cancer Cell Lines and a Normal Cell Line by MTT Assay

Cells were treated with Test Items at various concentrations (0.0001 μM-10 μM) for 48 h. The cells corresponding to Positive Control group were treated with Doxorubicin at a concentration ranging from 0.1 μM-100 μM. The Untreated cells were used as Control. After 48 h of incubation, the effect of Test Items on cytotoxicity of cells was determined by MTT assay, the plates were taken out and 20 μl of 5 mg/m1 of MTT 3-(4,5-dimethythiazol-2-yl)-2,5-diphenyl tetrazolium bromide solution was added to all the wells. Cells were incubated for 3 h at 37° C. The supernatant was aspirated. 150 μl of DMSO was added to each well to dissolve formazan crystals. The absorbance of each well was then read at 540 nm using Synergy HT micro plate reader.

The % Cytotoxicity was calculated using the formula:

$$\% Cytotoxicity = [ ( R - X ) / R ] * 100$$

Where, X=Absorbance of cells treated with Test Item; R=Absorbance of Control cells (Untreated cells maintained in growth medium only).

The IC50 value was calculated as the concentration leading to 50% inhibition of cell growth with respect to untreated cells using Graph pad prism version 4.01.

Selectivity Index was calculated using the formula:

SI=IC50 in Normal cell/IC50 in cancer cell.

Activity scores of the Selective Index values were categorized as:
Not selective (SI value ≤1)
Moderately selective (1<SI value <5)
Selective (SI value ≥5)

Results obtained were as follows:

TABLE 4a
CYTOTOXIC EFFECT OF SEQ ID No. 2, MMAE AND FORMULA
15 (DRUG CONJUGATE) AFTER 48 HOURS OF TREATMENT

| | Doxorubicin | | | | | | FORMULA 15 | |
|---|---|---|---|---|---|---|---|---|
| | (μm) | | | | | | (DRUG | |
| | Positive control | | SEQ ID No. 2 | | | | CONJUGATE) | |
| | (μm) | | (μm) | | MMAE (μm) | | (μm) | |
| | | Percent | | Percent | | Percent | | Percent |
| | | cytotoxicity | | cytotoxicity | | cytotoxicity | | cytotoxicity |
| Cell | | wrt | | wrt | | wrt | | wrt |
| Lines | Concentration | control | Concentration | control | Concentration | control | Concentration | control |
| MG-63 | 0.1 | 35.3 | 0.0001 | 27 | 0.0001 | 81.7 | 0.0001 | 33.2 |
| Bone | 1 | 60.7 | 0.001 | 38.7 | 0.001 | 85.5 | 0.001 | 16.7 |
| | 10 | 82 | 0.01 | 50.4 | 0.01 | 86.2 | 0.01 | 58.5 |
| | 50 | 85.5 | 0.1 | 59.1 | 0.1 | 87.4 | 0.1 | 77.1 |
| | 100 | 75.5 | 1 | 72.3 | 1 | 85.3 | 1 | 82.5 |
| | — | — | 10 | 74 | 10 | 82.3 | 10 | 85.6 |
| PA-1 | 0.1 | 19.6 | 0.0001 | 0.9 | 0.0001 | 13.4 | 0.0001 | 25.3 |
| Ovary | 1 | 62.6 | 0.001 | −5.6 | 0.001 | 57.5 | 0.001 | 20.7 |
| | 10 | 73.2 | 0.01 | 7.4 | 0.01 | 50 | 0.01 | 49.3 |
| | 50 | 79 | 0.1 | 4.5 | 0.1 | 57.5 | 0.1 | 45.9 |
| | 100 | 71.5 | 1 | 51 | 1 | 61.7 | 1 | 50.3 |
| | — | — | 10 | 72.2 | 10 | 58.3 | 10 | 73.9 |
| KB | 0.1 | 28.7 | 0.0001 | −1.5 | 0.0001 | 90.5 | 0.0001 | −7 |
| Cervix | 1 | 53.1 | 0.001 | −2.6 | 0.001 | 95.8 | 0.001 | −10.8 |
| | 10 | 95.7 | 0.01 | −3.9 | 0.01 | 96.1 | 0.01 | 9.4 |
| | 50 | 91.7 | 0.1 | 20.4 | 0.1 | 96.3 | 0.1 | 48.2 |
| | 100 | 84.2 | 1 | 51.2 | 1 | 96.2 | 1 | 84.3 |
| | — | — | 10 | 54.9 | 10 | 96.3 | 10 | 90.4 |
| T24 | 0.1 | 21 | 0.0001 | 9.7 | 0.0001 | 51.8 | 0.0001 | 11.8 |
| Bladder | 1 | 34.8 | 0.001 | 11.1 | 0.001 | 51.1 | 0.001 | 6.3 |
| | 10 | 72.8 | 0.01 | 9.1 | 0.01 | 46.1 | 0.01 | 5.5 |
| | 50 | 66.6 | 0.1 | 10.5 | 0.1 | 44.4 | 0.1 | 41.6 |
| | 100 | 52.7 | 1 | 43.1 | 1 | 44.4 | 1 | 47.9 |
| | — | — | 10 | 68.7 | 10 | 53.2 | 10 | 55 |
| MDA-MB- | 0.1 | 3.4 | 0.0001 | 13.7 | 0.0001 | 11.3 | 0.0001 | 9.1 |
| 231 | 1 | 68 | 0.001 | 15.1 | 0.001 | 13.3 | 0.001 | 6.9 |
| Breast | 10 | 74.3 | 0.01 | 16.2 | 0.01 | 28.9 | 0.01 | 19.4 |
| | 50 | 71.3 | 0.1 | 28.1 | 0.1 | 23.8 | 0.1 | −3.4 |
| | 100 | 58 | 1 | 48.5 | 1 | 16.8 | 1 | 53.1 |
| | — | — | 10 | 66.4 | 10 | 26.4 | 10 | 57.7 |
| U251 | 0.1 | −13.5 | 0.0001 | −9.9 | 0.0001 | 77.8 | 0.0001 | −14.8 |
| MG | 1 | 41.7 | 0.001 | −13.2 | 0.001 | 52.5 | 0.001 | −16.8 |
| Brain | 10 | 57.9 | 0.01 | −14.5 | 0.01 | 58.6 | 0.01 | 15.2 |
| | 50 | 71 | 0.1 | −5.3 | 0.1 | 63.1 | 0.1 | 14 |
| | 100 | 65.6 | 1 | 15.9 | 1 | 64.7 | 1 | 28.4 |
| | — | — | 10 | 62.5 | 10 | 71 | 10 | 72.6 |
| HT29 | 0.1 | 3.2 | 0.0001 | 14 | 0.0001 | 44.1 | 0.0001 | 5.4 |
| Colon | 1 | 1.7 | 0.001 | 16.6 | 0.001 | 44.6 | 0.001 | 2.4 |
| | 10 | 32.7 | 0.01 | 22.5 | 0.01 | 50.6 | 0.01 | 37.2 |
| | 50 | 61.9 | 0.1 | 24.7 | 0.1 | 50.4 | 0.1 | 52.8 |
| | 100 | 59.8 | 1 | 35.4 | 1 | 51.9 | 1 | 63.9 |
| | — | — | 10 | 54.1 | 10 | 55.9 | 10 | 68.2 |
| AGS | 0.1 | 15.6 | 0.0001 | 7.8 | 0.0001 | 30.9 | 0.0001 | 1.6 |
| Stomach | 1 | 72.4 | 0.001 | 8.5 | 0.001 | 31.7 | 0.001 | 39.2 |
| | 10 | 74.5 | 0.01 | 15.9 | 0.01 | 32.6 | 0.01 | 42.4 |
| | 50 | 74.8 | 0.1 | 37 | 0.1 | 48.2 | 0.1 | 49.2 |
| | 100 | 79 | 1 | 49.9 | 1 | 48.4 | 1 | 56.8 |
| | — | — | 10 | 57.2 | 10 | 54.4 | 10 | 67.6 |
| PANC-1 | 0.1 | −3.4 | 0.0001 | 9.5 | 0.0001 | 38.1 | 0.0001 | 50.2 |
| Pancreatic | 1 | 18.6 | 0.001 | 17 | 0.001 | 41 | 0.001 | 53.1 |
| | 10 | 67.3 | 0.01 | 20.5 | 0.01 | 64.1 | 0.01 | 56.2 |
| | 50 | 71.6 | 0.1 | 22.5 | 0.1 | 67.7 | 0.1 | 65.5 |
| | 100 | 70.1 | 1 | 34.4 | 1 | 77.1 | 1 | 70.6 |
| | — | — | 10 | 36.4 | 10 | 69.7 | 10 | 72.5 |
| A549 | 0.1 | 3.3 | 0.0001 | −0.5 | 0.0001 | 27.9 | 0.0001 | −8.8 |
| Lungs | 1 | 10.2 | 0.001 | −15.5 | 0.001 | 39.3 | 0.001 | −8.9 |
| | 10 | 54.2 | 0.01 | −1.1 | 0.01 | 43.5 | 0.01 | 12.8 |
| | 50 | 75.4 | 0.1 | −6.9 | 0.1 | 58.8 | 0.1 | 39.1 |
| | 100 | 76.2 | 1 | 31.9 | 1 | 83.6 | 1 | 60.2 |
| | — | — | 10 | 48.2 | 10 | 75.8 | 10 | 76.7 |
| PBMC | 0.1 | 5.3 | 0.0001 | −7.4 | 0.0001 | −2.7 | 0.0001 | −7.6 |
| Normal | 1 | 29.3 | 0.001 | 19.9 | 0.001 | 29.8 | 0.001 | 24.7 |
| | 10 | 37.4 | 0.01 | 24 | 0.01 | 34.3 | 0.01 | 28.1 |
| | 50 | 40.5 | 0.1 | 44.9 | 0.1 | 34.1 | 0.1 | 40.7 |
| | 100 | 37.1 | 1 | 49.5 | 1 | 26.6 | 1 | 57.2 |
| | — | — | 10 | 43.4 | 10 | 32.5 | 10 | 53.7 |

Conclusion:
The compound of FORMULA 15 (DRUG CONJUGATE) showed cytotoxicity towards panel of cell-lines tested.

TABLE 4b
CYTOTOXIC EFFECT OF TESIRINE AND FORMULA 17 (DRUG CONJUGATE) AFTER 48 HOURS OF TREATMENT

| | | | FORMULA 17 | | FORMULA 17 | | FORMULA 17 | |
|---|---|---|---|---|---|---|---|---|
| | | | (DRUG | | (DRUG | | (DRUG | |
| | | | CONJUGATES) | | CONJUGATES) | | CONJUGATES) | |
| | TESIRINE (μm) | | 2A (μm) | | 2B (μm) | | 2C (μm) | |
| | | Percent | | Percent | | Percent | | Percent |
| | | cytotoxicity | | cytotoxicity | | cytotoxicity | | cytotoxicity |
| Cell | | wrt | | wrt | | wrt | | wrt |
| Lines | Concentration | control | Concentration | control | Concentration | control | Concentration | control |
| MG-63 | 0.0001 | 46.6 | 0.0001 | 56.8 | 0.0001 | 28.6 | 0.0001 | 42.7 |
| Bone | 0.001 | 69.4 | 0.001 | 50.5 | 0.001 | 33.3 | 0.001 | 45.9 |
| | 0.01 | 81.2 | 0.01 | 53.4 | 0.01 | 31.3 | 0.01 | 28 |
| | 0.1 | 79.9 | 0.1 | 88.8 | 0.1 | 72.4 | 0.1 | 46.1 |
| | 1 | 82.3 | 1 | 91.7 | 1 | 89.6 | 1 | 90.7 |
| | 10 | 81.8 | 10 | 91.6 | 10 | 86 | 10 | 90.4 |
| PA-1 | 0.0001 | 53.1 | 0.0001 | 26.1 | 0.0001 | 10.2 | 0.0001 | 17.8 |
| Ovary | 0.001 | 66.8 | 0.001 | 36.4 | 0.001 | 18.4 | 0.001 | 25.2 |
| | 0.01 | 79.1 | 0.01 | 75.1 | 0.01 | 56.2 | 0.01 | 61.2 |
| | 0.1 | 79.9 | 0.1 | 80 | 0.1 | 80.3 | 0.1 | 80.6 |
| | 1 | 78.1 | 1 | 79.9 | 1 | 78.9 | 1 | 79.6 |
| | 10 | 80.5 | 10 | 80.7 | 10 | 80.2 | 10 | 80.3 |
| KB | 0.0001 | −9.5 | 0.0001 | 15.3 | 0.0001 | 15.7 | 0.0001 | −2 |
| Cervix | 0.001 | 1 | 0.001 | 27.1 | 0.001 | 17.6 | 0.001 | 11.5 |
| | 0.01 | 76.2 | 0.01 | 57.6 | 0.01 | 36.5 | 0.01 | 30.1 |
| | 0.1 | 93.8 | 0.1 | 95.5 | 0.1 | 91.2 | 0.1 | 90.2 |
| | 1 | 89.1 | 1 | 95.7 | 1 | 95.8 | 1 | 95.9 |
| | 10 | 92.7 | 10 | 95.9 | 10 | 94.2 | 10 | 93 |
| T24 | 0.0001 | 57.5 | 0.0001 | 21.6 | 0.0001 | 21.8 | 0.0001 | 24.3 |
| Bladder | 0.001 | 60.6 | 0.001 | 28.7 | 0.001 | 26.7 | 0.001 | 22.4 |
| | 0.01 | 57.5 | 0.01 | 33.7 | 0.01 | 26.3 | 0.01 | 23.3 |
| | 0.1 | 61.4 | 0.1 | 51 | 0.1 | 44.8 | 0.1 | 40.2 |
| | 1 | 62.7 | 1 | 57.7 | 1 | 55.4 | 1 | 59.6 |
| | 10 | 80.4 | 10 | 59.4 | 10 | 66.2 | 10 | 74.4 |
| MDA-MB- | 0.0001 | 45.9 | 0.0001 | 14.5 | 0.0001 | 58.5 | 0.0001 | 65.4 |
| 231 | 0.001 | 41.9 | 0.001 | −0.6 | 0.001 | 62.9 | 0.001 | 67.8 |
| Breast | 0.01 | 58.9 | 0.0 | 1.7 | 0.01 | 62.8 | 0.01 | 63.8 |
| | 0.1 | 68.6 | 0.1 | 44.7 | 0.1 | 68.3 | 0.1 | 69.8 |
| | 1 | 69.2 | 1 | 55.3 | 1 | 75.4 | 1 | 74.3 |
| | 10 | 80.7 | 10 | 77.6 | 10 | 73.7 | 10 | 81.8 |
| U251 | 0.0001 | −10.3 | 0.0001 | −1 | 0.0001 | 10.2 | 0.0001 | 11.5 |
| MG | 0.001 | 26.5 | 0.001 | 16.4 | 0.001 | 9.7 | 0.001 | 6.8 |
| Brain | 0.01 | 23.1 | 0.01 | 75.5 | 0.01 | 18.1 | 0.01 | 18.5 |
| | 0.1 | 19.2 | 0.1 | 62.6 | 0.1 | 77.9 | 0.1 | 77.4 |
| | 1 | 27.2 | 1 | 54.7 | 1 | 65.8 | 1 | 67.1 |
| | 10 | 60.5 | 10 | 70.3 | 10 | 77.3 | 10 | 78.6 |
| HT29 | 0.0001 | 37.4 | 0.0001 | 17.5 | 0.0001 | 5.7 | 0.0001 | 12.8 |
| Colon | 0.001 | 44.5 | 0.001 | 20.9 | 0.001 | 6.9 | 0.001 | 11.6 |
| | 0.01 | 47.7 | 0.0 | 22.9 | 0.01 | 15.8 | 0.01 | 10.7 |
| | 0.1 | 48.3 | 0.1 | 30.1 | 0.1 | 15.6 | 0.1 | 26.2 |
| | 1 | 49 | 1 | 53.2 | 1 | 39.2 | 1 | 43.5 |
| | 10 | 80.7 | 10 | 63.9 | 10 | 59.5 | 10 | 68.6 |
| AGS | 0.0001 | 66.4 | 0.0001 | 7.6 | 0.0001 | 27.7 | 0.0001 | 22.6 |
| Stomach | 0.001 | 77.5 | 0.001 | 3.2 | 0.001 | 25.3 | 0.001 | 19.9 |
| | 0.01 | 77.8 | 0.01 | 59.2 | 0.01 | 28.1 | 0.01 | 38.3 |
| | 0.1 | 81.3 | 0.1 | 69.6 | 0.1 | 73.3 | 0.1 | 71.7 |
| | 1 | 81.3 | 1 | 72.1 | 1 | 75 | 1 | 75.1 |
| | 10 | 81.6 | 10 | 78.8 | 10 | 71.6 | 10 | 78.4 |
| PANC-1 | 0.0001 | 46.6 | 0.0001 | 13.5 | 0.0001 | −2 | 0.0001 | 9.3 |
| Pancreatic | 0.001 | 51.1 | 0.001 | 11.4 | 0.001 | 24 | 0.001 | 6.5 |
| | 0.01 | 58.3 | 0.01 | 17.1 | 0.01 | 18.1 | 0.01 | 6.4 |
| | 0.1 | 55 | 0.1 | 35.7 | 0.1 | 21.6 | 0.1 | 18.7 |
| | 1 | 59.2 | 1 | 67.8 | 1 | 13.2 | 1 | 14.6 |
| | 10 | 71 | 10 | 77.5 | 10 | 58.9 | 10 | 68.6 |
| A549 | 0.0001 | 13.7 | 0.0001 | −6.3 | 0.0001 | −10.3 | 0.0001 | −3.8 |
| Lungs | 0.001 | 15.2 | 0.001 | −3.1 | 0.001 | −15.4 | 0.001 | −19.1 |
| | 0.01 | 34.4 | 0.01 | 12 | 0.01 | −2 | 0.01 | 1 |
| | 0.1 | 45.2 | 0.1 | 84.3 | 0.1 | 11.6 | 0.1 | 9.1 |
| | 1 | 53.3 | 1 | 75.2 | 1 | 38.4 | 1 | 68.6 |
| | 10 | 66.7 | 10 | 79.7 | 10 | 78.5 | 10 | 70.8 |
| PBMC | 0.0001 | 16.9 | 0.0001 | −4.4 | 0.0001 | 9.1 | 0.0001 | 29.1 |
| Normal | 0.001 | 36.5 | 0.001 | 22.9 | 0.001 | 14 | 0.001 | 25.5 |
| | 0.01 | 40.3 | 0.01 | 18.8 | 0.01 | 30.2 | 0.01 | 18.2 |
| | 0.1 | 38.7 | 0.1 | 45.9 | 0.1 | 34.4 | 0.1 | 33.1 |
| | 1 | 38.2 | 1 | 42.7 | 1 | 68.8 | 1 | 65.9 |
| | 10 | 55.3 | 10 | 65.7 | 10 | 68.5 | 10 | 70.4 |

Conclusion:
The compound of Formula 17 showed cytotoxicity towards panel of cell-lines tested.

TABLE 5
IC50 VALUES OF Formula 15 AND Formula 17 (RECOMBINANT-
PROTEIN DRUG CONJUGATES) AFTER 48 H OF TREATMENT

| | | | | | | | Formula | Formula | Formula |
|---|---|---|---|---|---|---|---|---|---|
| | | | SEQ | | Formula | | 17 | 17 | 17 |
| | | Doxorubicin | ID | | 15 | | Conjugate | Conjugate | Conjugate |
| | Cell | PC | NO. 2 | | Conjugate | TESIRINE | 2A | 2B | 2C |
| | Lines | (μm) | (μm) | MMAE(μm) | (μm) | (μm) | (μm) | (μm) | (μm) |
| IC50 | MG-63 | 0.4 | 0.0127 | 0.0001 | 0.0067 | 0.00003 | 0.00021 | 0.012 | 0.009 |
| Values | Bone | | | | | | | | |
| | PA-1 | 1 | 1.535 | 0.03011 | 0.1422 | 5.99E−06 | 0.002 | 0.011 | 0.007 |
| | Ovary | | | | | | | | |
| | KB | 0.6 | 0.88 | 0.0001 | 0.12 | 0.006 | 0.005 | 0.014 | 0.019 |
| | Cervix | | | | | | | | |
| | T24 | 5.5 | 2.13 | 0.00001 | 2.19 | 0.002 | 0.27 | 0.43 | 0.34 |
| | Bladder | | | | | | | | |
| | MDA-MB- | 2.1 | 1.35 | >10 | 1.58 | 0.0014 | 0.87 | 7.7DE−07 | 7.96E−09 |
| | 231 | | | | | | | | |
| | Breast | | | | | | | | |
| | U251 | 8.1 | 5.95 | 0.0002 | 2.82 | 6.82 | 0.04 | 0.07 | 0.07 |
| | MG | | | | | | | | |
| | Brain | | | | | | | | |
| | HT29 | 35.3 | 9.39 | 0.05 | 0.18 | 0.02 | 1.09 | 3.87 | 1.63 |
| | Colon | | | | | | | | |
| | AGS | 4.3 | 1.72 | 1.54 | 0.14 | 4.33E−10 | 0.03 | 0.03 | 0.02 |
| | Stomach | | | | | | | | |
| | PANC-1 | 8.2 | >10 | 0.0015 | 0.001 | 0.0007 | 0.31 | 1 | 0.44 |
| | Pancreatic | | | | | | | | |
| | A549 | 11 | 10 | 0.01 | 0.45 | 0.39 | 0.04 | 1.83 | 0.78 |
| | Lungs | | | | | | | | |
| | PBMC | >100 | >10 | >10 | 0.94 | 5.09 | 0.96 | 0.21 | 0.49 |
| | Normal | | | | | | | | |

Conclusion: PDC conjugates showed superior cytotoxic activity in all cancer types tested compared to Seq. ID. 2. The IC 50 values PDCs are very low in cancer cells when compared to normal cells.

TABLE 6
SELECTIVITY INDEX OF Formula 15 AND Formula 17 (RECOMBINANT-
PROTEIN DRUG CONJUGATES) AFTER 48 H OF TREATMENT WRT PBMCs

| | | | | Formula | | Formula | Formula | Formula |
|---|---|---|---|---|---|---|---|---|
| | | SEQ | | 15 | | 17 | 17 | 17 |
| | | ID | | Conjugate | | Conjugate | Conjugate | Conjugate |
| | Cell | NO. 2 | MMAE | 1A | TESIRINE | 2A | 2B | 2C |
| | Lines | (μm) | (μm) | (μm) | (μm) | (μm) | (μm) | (μm) |
| Selectivity | MG-63 | 786.78 | 100000 | 140.3 | 169666.7 | 4541.2 | 18.26 | 56.4 |
| wrt | Bone | | | | | | | |
| PBMC | PA-1 | 6.51 | 332.1 | 6.6 | 849182.5 | 449.9 | 19.14 | 73.2 |
| | Ovary | | | | | | | |
| | KB | 11.33 | 100000 | 7.9 | 844.8 | 193 | 14.89 | 26.1 |
| | Cervix | | | | | | | |
| | T24 | 4.69 | 1062837 | 0.4 | 2412.3 | 3.6 | 0.49 | 1.4 |
| | Bladder | | | | | | | |
| | MDA-MB- | 7.39 | 1 | 0.6 | 3707.2 | 1.1 | 272762.7 | 61573259.6 |
| | 231 | | | | | | | |
| | Breast | | | | | | | |
| | U251 | 1.68 | 49019.6 | 0.3 | 0.7 | 23.4 | 2.93 | 7.1 |
| | MG | | | | | | | |
| | Brain | | | | | | | |
| | HT29 | 1.07 | 208.4 | 5.1 | 236.1 | 0.9 | 0.05 | 0.3 |
| | Colon | | | | | | | |
| | AGS | 5.81 | 1 | 6.7 | 1.18E+10 | 37.7 | 7.04 | 20.7 |
| | Stomach | | | | | | | |
| | PANC-1 | 1 | 6662.2 | 987.8 | 7829.6 | 3.1 | 0.21 | 1.1 |
| | Pancreatic | | | | | | | |
| | A549 | 1 | 887.3 | 2.1 | 13 | 26.8 | 0.11 | 0.6 |
| | Lungs | | | | | | | |

Selectivity Index (SI) is interpreted as SI Value < 1 = Not selective; 1 < SI Value < 5 = Moderately selective; SI Value > 5 = Selective;
Conclusion:
PDC conjugates showed high selectivity index in cancer types tested compared to normal cells.

### Example 13

### In Vivo Anti-Tumor Potential of Compound of Formula 16 was Evaluated Using T24 Human Bladder Carcinoma Xenograft Model

In vivo anti-tumor potential of compound of Formula 16 was evaluated using T24 human bladder carcinoma xenograft model in mice by assessing biological endpoints such as tumor growth and tumor growth inhibition.

Thirty-five healthy female nude mice bearing T24 tumor with volume of ˜100 mm3 were selected, randomized and allocated into five groups (G1-G5, n=7 animals per group). Body weight of each mouse was recorded on the day of randomization and daily till the end of the study. The tumor size was measured weekly twice from the day of test item/reference drug/vehicle dosing till the end of the experiment. Tumor size was measured by digital caliper (MITUTOYO, Japan) by measuring length and width. Body weight and tumor volume data was statistically analysed using two-way ANOVA (Bonferroni Test). Statistical analysis was conducted using PRISM software, version 5.01.p values of <0.05 were considered as statistically significant. Percentage survival proportions were analyzed by “Kaplan Meier” method.

### Results:

TABLE 7

| | Percent | | | | | |
|---|---|---|---|---|---|---|
| | body | Tumor | | | Tumor | |
| | weight | volume | Percent | Percent | growth | |
| Group and | change | Day 22 | TGI | T/C | delay | Percent |
| treatment | Day 22 | (cu · mm) | Day 22 | Day 22 | (days) | survival |
| G1, | 5.67 | 1052.0 | 0.00 | 100.00 | NA | 100 |
| Vehicle | | | | | | |
| control | | | | | | |
| (5 mL/kg) | | | | | | |
| G2, | 6.3 | 622.6 | 40.82 | 59.18 | 10.5 | 100 |
| Cisplatin | | | | | | |
| (5 mg/kg), | | | | | | |
| Weekly | | | | | | |
| once | | | | | | |
| G3, | 4.73 | 540.6 | 48.62 | 51.38 | 7.5 | 100 |
| Formula 16 | | | | | | |
| (0.5 mg/kg), | | | | | | |
| Alternate | | | | | | |
| days | | | | | | |
| G4, | 1.53 | 705.2 | 32.96 | 67.04 | 6 | 100 |
| Formula | | | | | | |
| 16 | | | | | | |
| (1 mg/kg), | | | | | | |
| once in | | | | | | |
| three days | | | | | | |
| G5, | 5.84 | 859.8 | 18.27 | 81.73 | 3 | 100 |
| Formula 16 | | | | | | |
| (2 mg/kg), | | | | | | |
| once in six | | | | | | |
| days | | | | | | |

Abbreviation
TGI: Tumor Growth Inhibition
T/C: Test/Control

### Conclusion

The biological end points such as mean tumor volume, tumor growth inhibition (TGI) and % o Test/Control (ST/C) showed that the treatment of Formula 16 at the given doses and regimen showed reduction in tumor volume in T24 xenograft model without body weight loss. Statistically significant anti-tumor activity was observed at the doses of 0.5 mg/kg, alternate days (p<3.001; TGL48.62%) on Day 22 when compared to vehicle control group GD. Hence it can be concluded based on the study conducted that the Protein drug conjugate Formula 16 showed invivo anti-tumor efficacy in human urinary bladder carcinoma xenograft model.

### Example 14: Histopathological Investigation of Tumour Tissue

Effect of compound of Formula 16 was evaluated using T24 human bladder carcinoma xenograft model.

TABLE 8

| Group and treatment | Apoptosis | Grade | |
|---|---|---|---|
| G1, Vehicle control | Mild | 3 | Moderately |
| (5 mL/kg) | | | Differentiated |
| G2, Cisplatin | Mild | 2 | Moderately |
| (5 mg/kg), Weekly once | | | Differentiated |
| G3, Formula 16(0.5 mg/ | Moderate | 2 | Moderately |
| kg), Alternate days | | | Differentiated |
| G4, Formula 16(1 mg/ | Moderate | 2 | Moderately |
| kg),once in three days | | | Differentiated |
| G5, Formula 16 (2 mg/ | Marked | 1 | Well |
| kg), once in six days | | | Differentiated |

Note:
Low grade indicates a better apoptosis.
Grade 1 (Well differentiated) - <25% cells anaplastic; Grade 2 (Moderately Differentiated) - <25-50% cells anaplastic; Grade 3 (Moderately Differentiated) - <50-75% cells anaplastic; Grade 4 (Poorly Differentiated) - >75% cells anaplastic

### Conclusion

Treatment Group (G3, G4, G5) showed
increased apoptosis, angiogenesis, low mitotic figures; and
lower grade as compared to vehicle group indicated anti tumour effect of compound of Formula 16 in T24 human bladder carcinoma xenograft model.

### Example 15: Effect of Compound of Formula 16 on Immune System

Effect of compound of Formula 16 was evaluated using T24 human bladder carcinoma xenograft model.

### Results:

TABLE 9
Effect of compound of Formula 16 on Immune System

| | Th1 cytokines | | | | | |
|---|---|---|---|---|---|---|
| | IFN | | Th2 cytokines | | Immunoglobulins | |
| Group and | gamma | IL-2 | IL-10 | IL-4 | IgG | IgM |
| Treatment | (pg/ML) | (pg/ml) | (pg/ml) | (pg/ml) | (ng/ml) | (ng/mL) |
| G1, Vehicle | 50.4 | 148.4 | BLQ | 68.7 | 54.6 | 39.5 |
| Control, 5 mL/kg | | | | | | |
| G2, Cisplatin, | 80.8 | 259.3 | BLQ | 60 | 54 | 18 |
| 5 mg/kg, i.p. | | | | | | |
| (q7dX2) | | | | | | |
| G3, | 54.8 | 182.5 | 652.9 | 63.4 | 56.7 | 24.6 |
| Formula 16 | | | | | | |
| 0.5 mg/kg, i.v. | | | | | | |
| (q2dX7) | | | | | | |
| G4, Formula 16 | 104.4 | 229.5 | 219.1 | 61.1 | 59.1 | 29.5 |
| 1 mg/kg, i.v. | | | | | | |
| (q3dX5) | | | | | | |
| G5,-Formula 16 | 73.3 | 220.8 | 331.7 | NA | 55.6 | 26.5 |
| 2 mg/kg, (q6dX3) | | | | | | |

### Conclusion

Th1 cytokines were found to be increased demonstrating immune cell activation for NK cell mediated suppression in tumour growth.
Cisplatin group showed reduction in IgM indicating immunosuppression.
Treatment group (G3, G4, G5) showed moderately altered level of IgM.

### Example 16: Evaluation of Acute Toxicity of Compound of Formula 16 in BALB/c Mice

Twenty-five healthy female BALB/c mice were used in the study. Animals were acclimatized for five days. After completion of acclimatization, animals were randomized based on the body weight and grouped into five groups (G1-G5), comprising five animals in each group. Animals from G1, G2, G3, G4 and G5 received Formula 16 intravenously once at the dose of 0.5 mg/kg, 2.5 mg/kg, 5 mg/kg, 7.5 mg/kg and 10 mg/kg respectively at a dose volume of 5 mL/kg. Animals were observed for clinical sign of toxicity and mortality once daily up to 14 days. Body weight was measured on day 1, 2, 3, 7 and 14. On day 15, animals were necropsied for gross pathological investigation of vital organs was carried out.

No clinical signs of toxicity were observed in G1 and G2 animals. In G3 group, 3 animals showed mild symptoms (Lethargy) whereas 2 animals showed severe symptoms (Lethargy, Tremor, Convulsion) however animals recovered after ˜15-20 minutes post dosing. In G4 group, 1 animal died immediately post dosing and necropsied for gross pathology, reddish coloration of all lobes of lungs was found. Whereas 4 animals showed severe symptoms (Lethargy, Tremor, Convulsion, Gasping) however animals recovered later. In G5 group, all animals died immediately post dosing and necropsied for gross pathology, reddish coloration of all lobes of lungs was observed. All the surviving animals from groups G1-G4 were necropsied on day 15 and no visible changes were found during the gross pathological examination of vital organs.

Based on the study, single i.v. dose of compound of Formula 16 at concentrations 0.5 mg/kg, 2.5 mg/kg, 5 mg/kg was found to be well tolerated and safe.

1. A protein drug conjugate comprising:
a) a lectin; and
b) a drug;
wherein lectin is conjugated to drug;
wherein lectin is a recombinant lectin having amino acid sequence of SEQ ID NO: 1; or the lectin having at least 70% identity to SEQ ID NO: 1.

2. The protein drug conjugate as claimed in claim 1, wherein the lectin has binding affinity towards one or more antigen selected from the group comprising of Thomsen-Friedenreich antigen (O-GalNAc Core1 antigen), and its extended core structures (Core2, α2,3/6-sialyl Core1 (Sialyl-T antigen), α2,6/6-sialyl Core2), and its modified forms.

3. The protein drug conjugate as claimed in claim 1, wherein the lectin having at least 70% identity to SEQ ID NO: 1 is selected from a group consisting of SEQ ID NO: 2, 3, 4, 5 and 8.

4. The protein drug conjugate as claimed in claim 1, wherein lectin is conjugated to drug by a covalent bond or a coordinate covalent bond.

5. The protein drug conjugate as claimed in claim 1, wherein lectin is attached to drug via free amino moiety (—NH— or —NH₂ group) or free thiol moiety (—SH group) or free acid (—COOH group) or free hydroxyl (OH group) available on amino acid.

6. The protein drug conjugate as claimed in claim 1, wherein the lectin is bound to the drug by a covalent bond or a coordinate covalent bond, via linker.

7. The protein drug conjugate as claimed in claim 1, wherein lectin is used for delivery of drug to the target cell.

8. The protein drug conjugate as claimed in claim 1, wherein drug is selected from a group comprising of therapeutic agent, cytotoxic agent, anti-cancer agent, diagnostic agent, and combinations thereof.

9. The protein drug conjugate as claimed in claim 1, wherein the drug is selected from a group comprising of monomethyl auristatin E (MMAE), Tesirine, DM1, DM4, DM 21, Doxorubicin, Govitecan, Dxd 1, and Amantin.

10. The protein drug conjugate as claimed in claim 1, wherein the lectin is conjugated to drug selected from MC-VC-PAB-MMAE, mc-hydrazone-Doxorubicin, DM21-C, DXd(1), SMCC-DM1, Govitecan (Linker-SN38), SPDB-DM4, mc-vc-PAB-C6-α-Amanitin, MC-GGFG-NH-CH2-Exatecan, Tesirine (Linker-SG3249).

11. The protein drug conjugate as claimed in claim 1, wherein the conjugate is used in treatment or prevention of cancer.

12. A pharmaceutical composition comprising:
a) a protein drug conjugate as claimed in claim 1; and
b) one or more pharmaceutically acceptable excipient(s).

13. The pharmaceutical composition as claimed in claim 12, wherein the said composition is used in treatment or prevention of cancer.

14. A protein drug conjugate of Formula I; 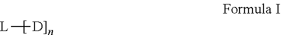
or a pharmaceutically acceptable salt thereof; wherein
L is lectin;
D is drug;
“—” between L and D is a bond; and
n is 1 to 9.

15. The protein drug conjugate as claimed in claim 14, wherein lectin is a recombinant lectin having amino acid sequence of SEQ ID NO: 1; or the lectin having at least 70% identity to SEQ ID NO: 1.

16. The protein drug conjugate as claimed in claim 14, wherein drug is selected from a group comprising of therapeutic agent, cytotoxic agent, anti-cancer agent, diagnostic agent, and combinations thereof.

17. The protein drug conjugate as claimed in claim 16, wherein the cytotoxic or therapeutic agent is selected from a group comprising of monomethyl auristatin E (MMAE), Tesirine, DM1, DM4, DM 21, Doxorubicin, Govitecan, Dxd 1, and Amantin.

18. The protein drug conjugate as claimed in claim 14, wherein drug is selected from an DNA binding agent, an anti-microtubule agent, an antimetabolite, an alkylating or alkylating-like antineoplastic agent, a topoisomerase I inhibitor, a topoisomerase II inhibitor, a kinase inhibitor, bortezomib, estramustine, Ixabepilone, everolimus, temsirolimus, neomycin, neamine, a cryptophycin, discodermolide, amanitin, or a pyrrolobenzodiazepine dimer, or a pharmaceutically acceptable salt, polymorph, and solvate thereof.

19. The protein drug conjugate as claimed in claim 18, wherein the DNA binding agent is selected from bleomycin, netropsin, distamycin or their analogues including lexitropsins, enediyne, mitomycin, and duocarmycin.

20. The protein drug conjugate as claimed in claim 18, wherein anti-microtubule agent is selected from auristatin, maytansine, and maytansinoid.

21. The protein drug conjugate as claimed in claim 14, wherein each drug is conjugated to L via:
a) a covalent bond, or
b) a group of formula “-A¹-A²-A³”;
wherein the other terminal of A¹ and A³ is connected to L and D respectively; and wherein A¹ and A³ is independently selected from: —CR¹R²—, —C(O)—, —C(O)NR¹—, —NR¹C(O)—, —NR¹—, —O—, —S—, —S(O)—, —S(O)₂, —R¹C═CR²—, —C≡C—, —R¹C═CR²—R³C═CR⁴—, —R¹C═C═CR²—, —C(═NR¹)—, 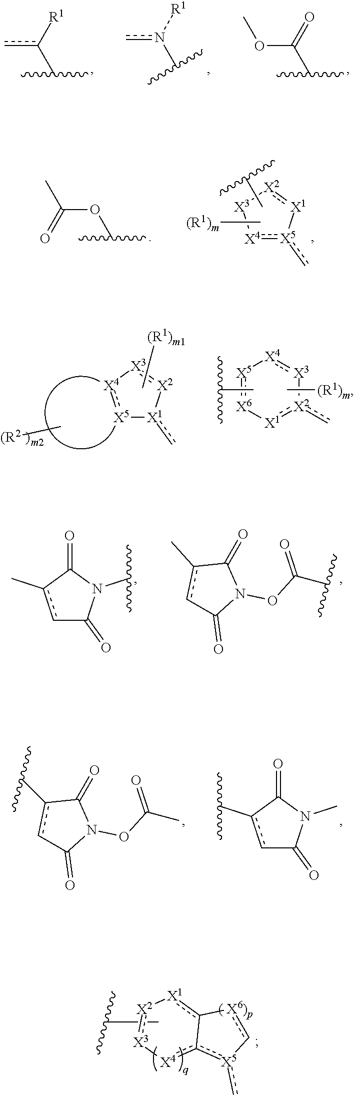
wherein
m, m1 and m2 are independently 0, 1, 2, 3, or 4; p is 1 or 2 and q is 0 or 1;
“ [symbol] ” indicates the point of attachment of A² to A¹ or A³;
“ [symbol] ” indicates single or double bond; 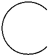
indicates a 5 to 10 membered ring including atoms of the ring it is fused to, wherein the 5 to 10 membered ring is an aromatic, non-aromatic, or cycloalkyl ring or a heteroaromatic or heterocycloalkyl ring wherein the heteroaromatic or heterocycloalkyl ring comprise at least one heteroatom selected from N, O or S;
each of X¹, X², X³, X⁴, X⁵ and X⁶ is independently selected from C, CH, CH₂, N, NH, O or S; and
R¹, R², R³ and R⁴ are independently selected from hydrogen, C₁ to C₅ alkyl, C₂ to C₅ alkenyl, —C₂ to C₅ alkynyl, C₁ to C₅ alkylene, C₃ to C₁₀ cycloalkyl, C₂ to C₉ heterocycloalkyl, C₆ to C₁₀ aryl, C₂ to C₉ heteroaryl, —OR⁵, —NR⁵—SR⁵, halogen selected from fluorine, chlorine, bromine or iodine, and —C(O)OR⁵, wherein C₁ to C₅ alkyl, C₂ to C₅ alkenyl, —C₂ to C₅ alkynyl, C₁ to C₅ alkylene, C₃ to C₁₀ cycloalkyl, C₂ to C₉ heterocycloalkyl, C₆ to C₁₀ aryl, and C₂ to C₉ heteroaryl are unsubstituted or are each further substituted by R⁶, and
R⁵ and R⁶ are each independently hydrogen, C₁ to C₃ alkyl, OH or halogen;
wherein A² is of formula: 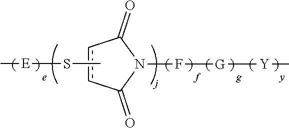
wherein
e, j, f, g and y are independently 0 or 1, provided e, f and y are not 0 simultaneously,
S is sulphur atom,
E, F, and Y is a spacer, and
G is an amino acid or a peptide chain comprising 2, 3, 4, 5, 6, 7, 8, 9, 10, 11 or 12 amino acids;
and wherein A¹ forms bond with E or S or F and A³ is connected to Y or G.

22. The protein drug conjugate as claimed in 21, wherein each spacer E, F and Y is independently selected from —R⁷—, —S—, —NH—, —R⁷—N—R⁸—, —R⁸—NC(O)—R⁷—, —R⁸—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, O—R⁷—, —R⁷—O, —R⁸—O—R⁷—, —C(O)O—, —OC(O)—R⁷—, —R⁷—OC(O)—, —R⁸—OC(O)—R⁷—, —R⁷—OC(O)O—, —OC(O)O—R⁷—, —R⁷—OC(O)O—R⁸—, —NH—R⁷—NH—C(O)R⁸—, —(CH2)S—NH—R⁷—, —R⁷—C(O)N(CH2)1-3O(CH2)1-3C(O)—, —R⁷—NC(O)(CH2)1-3O(CH2)1-3C(O)—, —Si(R⁷R⁸)—, polyalkylene glycol optionally attached through oxygen to —R⁷—, —S—, —NH—, —R⁷—N—R⁸—, —R⁸—NC(O)—R⁷—, —R⁸—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, S(O)—, —S(O)—R⁷—, —R⁷—S(O)—, —R⁷—S(O)—R⁸—, —O—R⁷—, —R⁷—O—, —R⁸—O—R⁷—, —C(O)O—, —OC(O)—R⁷—, —R⁷—OC(O)R⁸—OC(O)—R⁷—, —R⁷—OC(O)O—, —OC(O)O—R⁷—, —R⁷—OC(O)O—R⁸—, —NH—R⁷—NH C(O)R⁸—, (CH2)S—NH—R⁷—, —R⁷—C(O)N(CH2)1-3O(CH2)1-3C(O)—, —R⁷—NC(O)(CH2)1-3O(CH2)1-3C(O)—, —Si(R⁷R⁸)—;
wherein R⁷ and R⁸ are independently selected from hydrogen, —C1-C10 alkylene, —C1-C10 alkenylene, C5 to C10 saturated or unsaturated cycloalkylene, C2-C10 alkenylene-C5 to C10 saturated or unsaturated cycloalkylene, C6 to C10 arylene, —C2-C10 alkenylene-C6 to C10 arylene C2 to C9 heteroarylene, —C2-C10 alkenylene-C2 to C9 heteroarylene; and wherein the —C1-C10 alkylene, —C2-C10 alkenylene, C5 to C10 saturated or unsaturated cycloalkylene, —C2-C10 alkenylene-C5 to C10 saturated or unsaturated cycloalkylene, C6 to C10 arylene, —C2-C10 alkenylene-C6 to C10 arylene, C2 to C9 heteroarylene and —C2-C10 alkenylene-C2 to C9 heteroarylene is optionally further substituted with —OH, —C(O), —SH, —NH2, halogen, C1 to C5 alkyl, —NO2, or —CN;
wherein polyalkylene glycol is a homopolymer or copolymer of alkylene with each unit of alkylene having C1 to C8 carbon atoms and total number of alkylene units ranging from 1 to 20; and wherein when polyalkylene glycol is E the —CH2- end is attached to A1 and when polyalkylene glycol is F the —CH2- end is attached to —R⁷—, —S—, —NH, —R⁷—N—R⁸—, —R⁸—NC(O)—R⁷—, —R⁸—C(O)N—R⁷—, —C(O)—, —C(O)—R⁷—, —R⁷—C(O)—, —R⁸—C(O)—R⁷—, —S—R⁷—, —R⁷—S—, —R⁷—S—R⁸—, —S(O)—, —S(O)—R⁷—, —R⁷—S(O)—, —R⁷—S(O)—R⁸—, —O—R⁷—, —R⁷—O—, —R⁸—O—R⁷—, —C(O)O—, —OC(O)—R⁷—, —R⁷—OC(O)—, —R⁸—OC(O)—R⁷—, —R⁷—OC(O)O—, —OC(O)O—R⁷—, —R⁷—OC(O)O—R⁸—, —NH—R⁷—NH—C(O)R⁸— or (CH2)S—NH—R⁷—, —R⁷—C(O)N(CH2)1-3O(CH2)1-3C(O)— or R⁷—NC(O)(CH2)1-3O(CH2)1-3C(O)—.

23. A compound of Formula C represented as 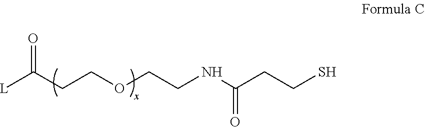
wherein
x is 8 or 12; and
L is a lectin having amino acid sequence of SEQ ID NO: 1; or the lectin having at least 70% identity to SEQ ID NO: 1.

24. The compound of Formula C as claimed in claim 23, wherein the compound of formula C is prepared by process comprising:

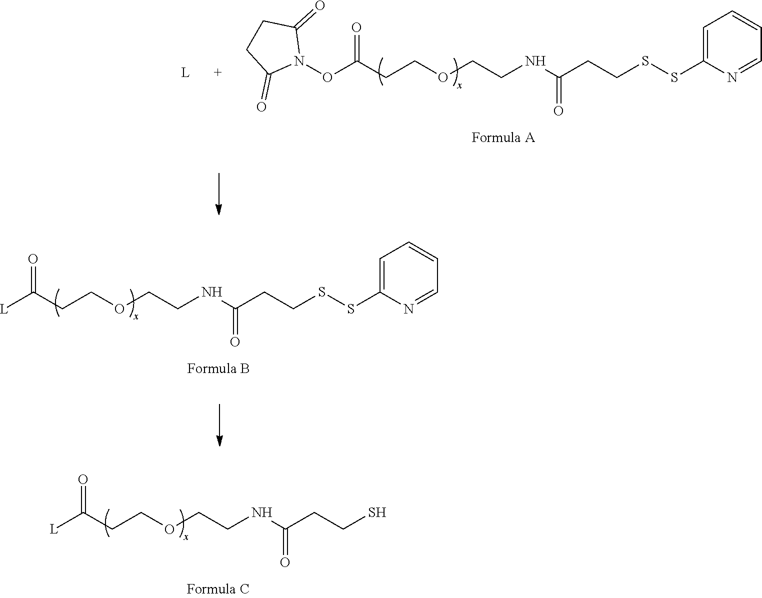

25. The protein drug conjugate as claimed in claim 14, wherein compound of is selected from Formula II, Formula III, and Formula IV:
L-D Formula II; 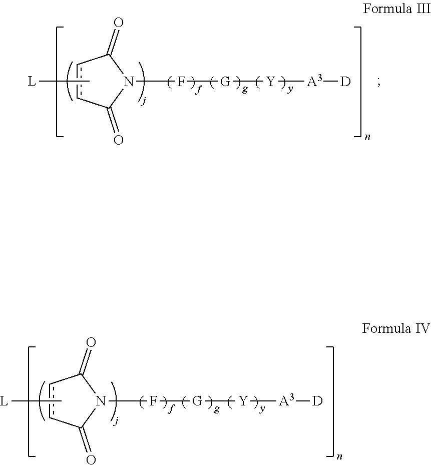
or a pharmaceutically acceptable salt thereof, wherein L, F, G, Y, A¹, A³, D, n, j, f, g, and y are as defined herein before.

26. A protein drug conjugate, selected from the group comprising of Formula 15, 16, 17, 18 and 19; 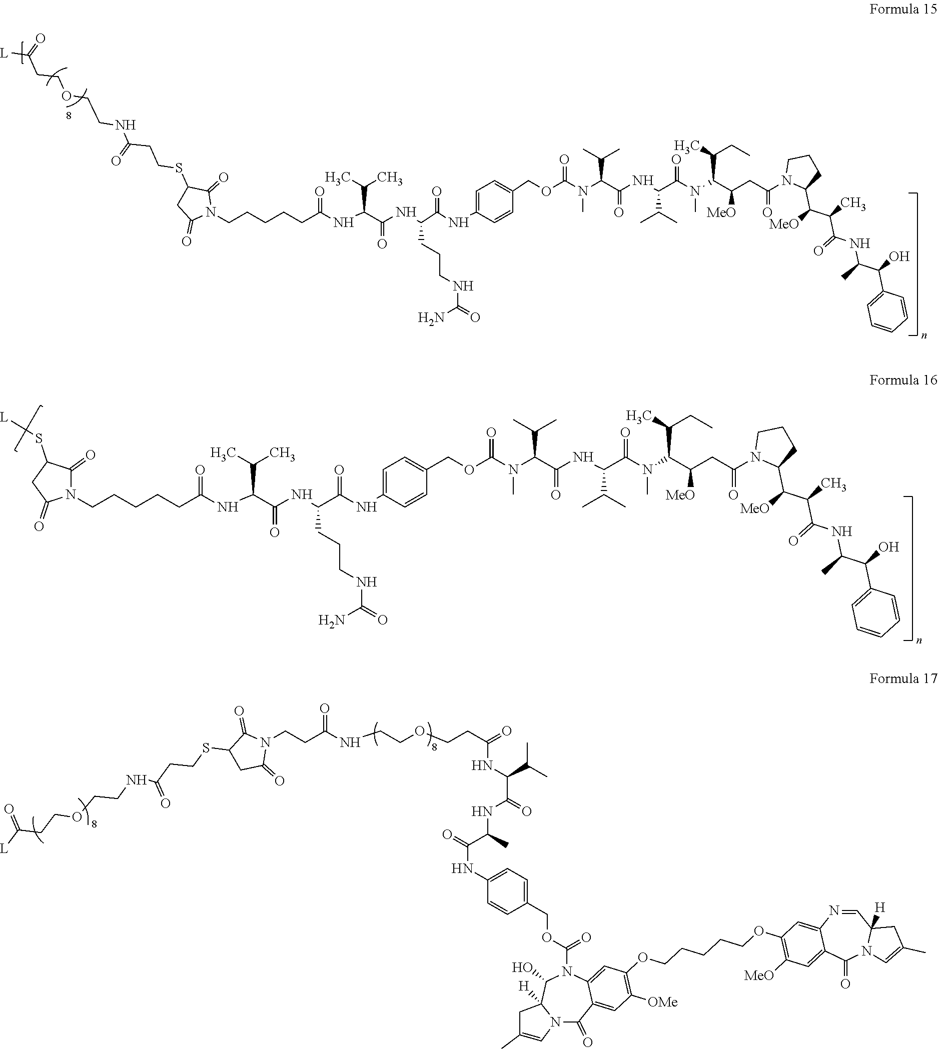 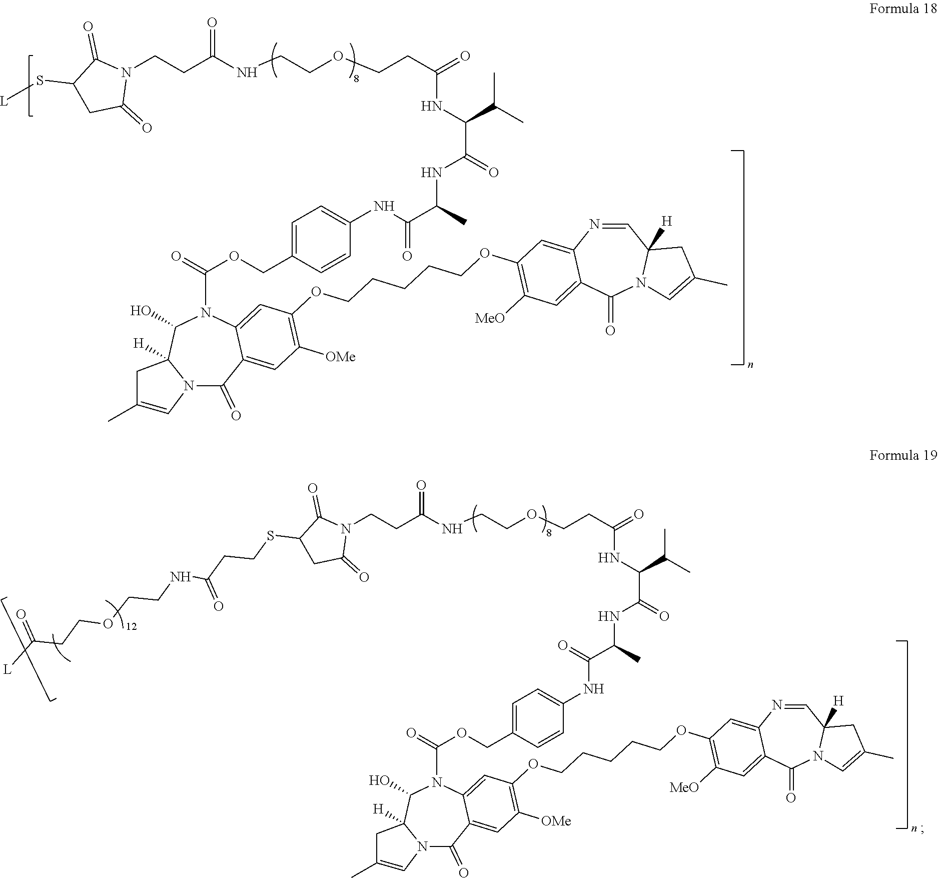
or a pharmaceutically acceptable salt or derivatives thereof,
L is a lectin having amino acid sequence of SEQ ID NO: 1; or the lectin having at least 70% identity to SEQ ID NO: 1.

27. The protein drug conjugate as claimed in claim 25, wherein
conjugate of Formula 15, 17 and 19 comprises of Lectin of Seq ID No. 2 conjugated to drug via an intermediate compound represented by Formula C; and
conjugate of Formula 16 & 18 comprises of Lectin of Seq ID No. 5 conjugated to drug via an Cysteine (SH bond) at carboxy terminal of Lectin of Seq ID No. 5.

28. The protein drug conjugate as claimed in claim 25, wherein the process for preparation of Formula 15 comprises: 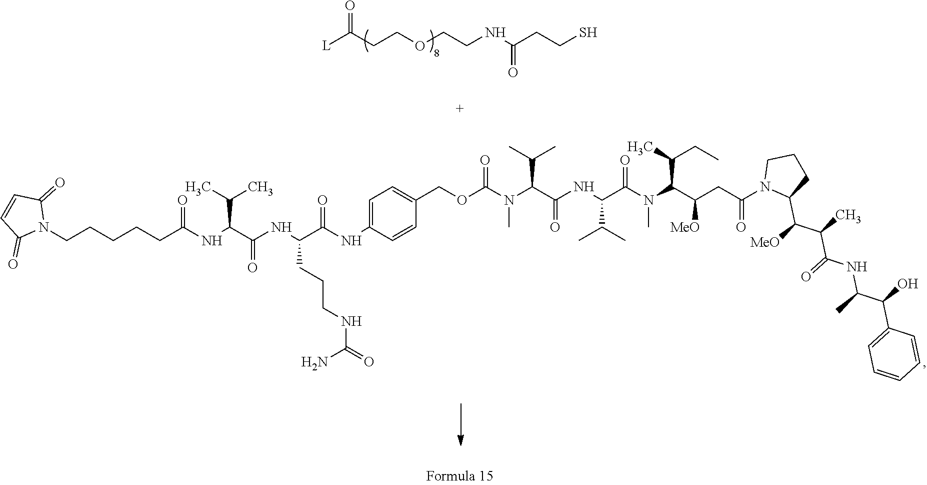 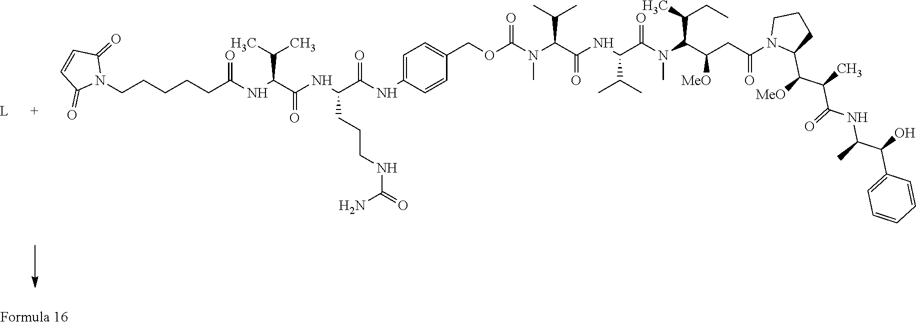 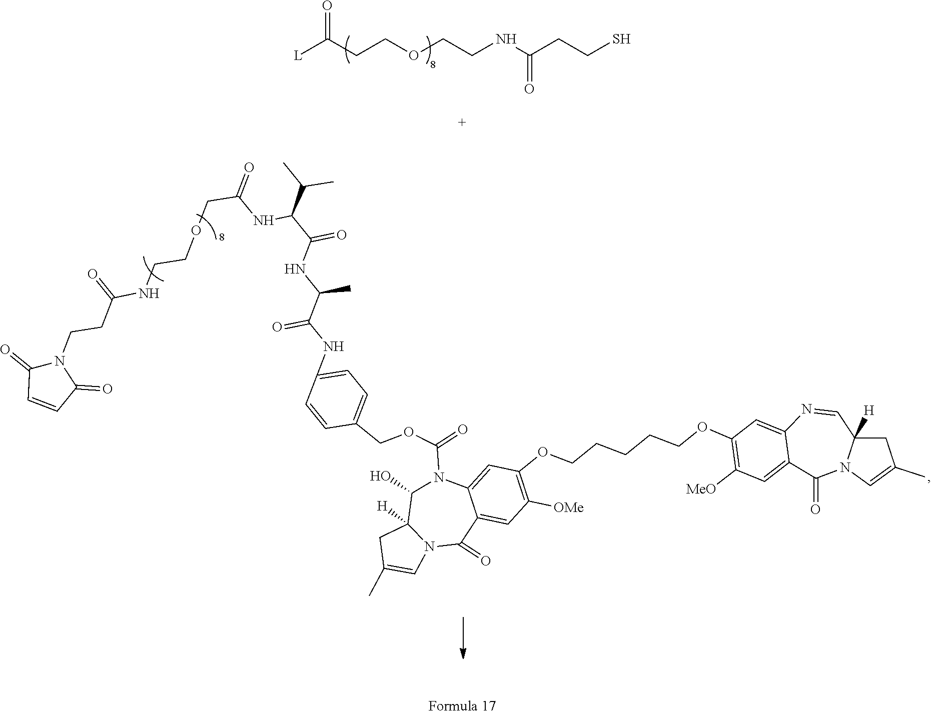 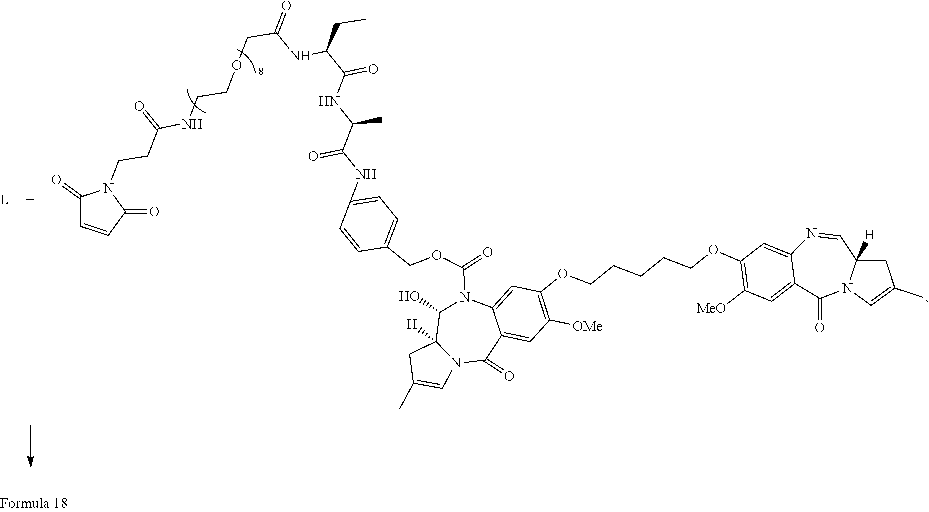 [chemical structure]


SEQUENCE LISTING (9 sequences)

SEQ ID NO: 1 - protein, 141 aa, sclerotium rolfsii
TYKITVRVYQTNPNAFFHPVEKTVWKYANGGTWTITDDQHVLTMGGSGTSGTLRFHADNGESFTATFGVHNYKRWCDIVTNLAADETGMVINQQYYSQKNREEARERQLSNYEVKNAKGRNFEIVYTEAEGNDLHANLIIG

SEQ ID NO: 2 - protein, 141 aa, synthetic construct
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 3 - protein, 141 aa, synthetic construct
VYKITVRVYQTNPDAFFHPVEKTVWKYANGGTWSITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 4 - protein, 141 aa, synthetic construct
VYKITVRVYQTNPDAFFHPVEKTVWKYADGGTWSITDDQHVLTMGGSGTSGTLRFHADNGESFTATFGVHDYKRWCDIVTDLAADETGMVINQEYYSEKDREEARERQNSNYEVKDAKGRNFEIVYTEAEGNDLHADLIIG

SEQ ID NO: 5 - protein, 142 aa, synthetic construct
SYKITVRVYQTNPDAFFHPVEKTVWKYANGGTWTITDDQHVLTMGGSGTSGTLRFHADNGESFTATFGVHNYKRWGDIVTNLAADETGMVINQQYYSQKNREEARERQLSNYQVKNAKGRNFQIVYTEAEGNDLHANLIIGC

SEQ ID NO: 6 - DNA, 426 bp, synthetic construct
  source 1..426: mol_type = other DNA
atgacctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggtgtgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggc

SEQ ID NO: 7 - DNA, 426 bp, synthetic construct
  source 1..426: mol_type = other DNA
agctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggctgc

SEQ ID NO: 8 - protein, 143 aa, synthetic construct
SYKITVRVYQTNPDAFFHPVEKTVWKYANGGTWTITDDQHVLTMGGSGTSGTLRFHADNGESFTATFGVHNYKRWGDIVTNLAADETGMVINQQYYSQKNREEARERQLSNYQVKNAKGRNFQIVYTEAEGNDLHANLIIGSC

SEQ ID NO: 9 - DNA, 429 bp, synthetic construct
  source 1..429: mol_type = other DNA
agctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggcagctgc